(19) United States
(12) Patent Application Publication
(10) Pub. No.: US 2026/0358341 A1
(43) Pub. Date: Nov. 12, 2026

(54) METHODS AND COMPOSITIONS FOR NON-INVASIVE RISK STRATIFICATION IN UVEAL MELANOMA USING AQUEOUS HUMOR PROTEOMIC PROFILING

(71) Applicant: The Saban Research Institute, Children's Hospital Los Angeles, Los Angeles, CA (US)

(72) Inventors: Jesse Berry, Manhattan Beach, CA (US); Liya Xu, San Diego, CA (US); Chen-Ching Peng, Los Angeles, CA (US)

(21) Appl. No.: 19/667,270

(22) Filed: May 4, 2026

Related U.S. Application Data
(60) Provisional application No. 63/799,629, filed on May 4, 2025.

(51) Int. Cl. G01N 33/5751; G16B 25/10; G16B 40/00

(52) U.S. Cl. CPC G01N 33/5751; G16B 25/10; G16B 40/00

(57) ABSTRACT

Provided are methods, compositions, and kits for non-invasive stratification of metastatic risk in subjects having uveal melanoma through proteomic profiling of aqueous humor. Protein expression levels are quantified and a machine learning classifier model is applied to a panel of protein biomarkers selected from a combinatorial biomarker group to generate a metastatic risk score. The classifier stratifies subjects as having high or low metastatic risk, including subjects lacking tumor-derived gene expression profile data. Multiple functionally equivalent biomarker subsets derived from the combinatorial group enable flexible assay deployment and reproducible risk assessment.

### PRIORITY

This application claims the benefit of the filing date of U.S. provisional application Ser. No. 63/799,629, filed on May 4, 2025, the disclosure of which is incorporated by reference herein.

### GOV FUNDING

This invention was made with government support under R41CA291243 awarded by the National Institutes of Health. The government has certain rights in the invention.

### FIELD OF THE INVENTION

The present disclosure relates to the field of oncology diagnostics, and more particularly to methods, compositions, and kits for non-invasive risk stratification of uveal melanoma through proteomic profiling of aqueous humor. The present disclosure further relates to biomarker-based methods for predicting metastatic risk in uveal melanoma, and more specifically to protein biomarkers measured in aqueous humor and their use in combinatorial predictive models for clinical risk stratification.

### BACKGROUND

Uveal melanoma (UM) is the most common primary intraocular malignancy in adults. Despite effective local control of the primary tumor through radiation therapy, enucleation, or other modalities, approximately half of patients develop systemic metastases, predominantly to the liver. Metastatic uveal melanoma remains largely incurable, with limited response to conventional systemic therapies, including immune checkpoint inhibitors that have demonstrated efficacy in cutaneous melanoma.

### SUMMARY

This invention relates to the field of oncology diagnostics and specifically to a method for stratifying metastatic risk in uveal melanoma using proteomic profiling of aqueous humor (AH) Uveal melanoma is the most common primary intraocular malignancy in adults, with a high risk of metastasis-most often to the liver. Gene expression profiling (GEP) of tumor biopsies is the current clinical standard for stratifying UM into low-risk (GEP1) or high-risk (GEP2) categories. However, biopsy procedures are invasive and carry significant risk, including vision loss, hemorrhage, tumor seeding, and sampling variability. There is a pressing need for safe, non-invasive alternatives for risk stratification.

Provided herein is a non-invasive diagnostic tool that uses proteomic profiling of aqueous humor (AH) to predict metastatic risk in uveal melanoma. A panel of proteins has been identified. These biomarkers demonstrate distinct expression patterns between low- and high-risk UM tumors, with several showing high diagnostic performance (e.g., AUC >0.7). The approach supports both absolute quantification and relative expression analysis and enables scalable, repeatable, and clinically meaningful liquid biopsy diagnostics.

The present disclosure relates to methods, compositions, and diagnostic kits for stratifying metastatic risk in subjects having uveal melanoma through proteomic profiling of aqueous humor. The invention provides a non-invasive alternative to tumor biopsy-based gene expression profiling (GEP) that is applicable across the clinical spectrum of uveal melanoma patients, including the substantial subset of patients who do not undergo fine-needle aspiration biopsy of the tumor.

In one aspect, the invention provides a method for stratifying metastatic risk in a subject having uveal melanoma. The method comprises obtaining an aqueous humor sample from the subject; quantifying protein expression levels in the aqueous humor sample using any suitable quantification/detection platform, including, but not limited to, a multiplex proximity extension assay, Meso Scale Discovery (electrochemiluminescence), Luminex multiplex immunoassay, ELISA, mass spectrometry, single molecule array (Simoa), or aptamer-based assay (e.g., SomaScan), to generate protein expression values for a plurality of proteins; applying a machine learning classifier model to the protein expression values of a panel of protein biomarkers to generate a metastatic risk score, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma; and classifying the subject as having high metastatic risk or low metastatic risk based on the metastatic risk score. The panel comprises at least three, at least five, or at least eight protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS. In some embodiments, the machine learning classifier model is trained on aqueous humor protein expression values from subjects with known gene expression profile classifications to discriminate between GEP1 (low metastatic risk) and GEP2 (high metastatic risk) uveal melanoma.

In certain embodiments, the aqueous humor sample is collected from the subject prior to any therapeutic intervention for uveal melanoma. In other embodiments, the aqueous humor sample is obtained by clear corneal paracentesis, providing a minimally invasive sample acquisition method. The assay, such as multiplex proximity extension, may generate both normalized protein expression values and extrapolated absolute concentration values in picograms per milliliter derived by regression-based calibration using quantitative polymerase chain reaction-based on reference concentration values. The multiplex proximity extension assay may be performed using a dual-platform calibration comprising a next generation sequencing-based analysis and a quantitative polymerase chain reaction-based analysis.

The invention provides a combinatorial biomarker framework from which multiple distinct and functionally equivalent biomarker subsets may be derived. In certain embodiments, the panel of protein biomarkers comprises PAEP, SMARCA2, FAM3B, KEL, SLITRK6, ANXA10, TNFRSF9, ADCYAP1R1, DECR1, GDF2, and PDCD1. In other embodiments, the panel comprises PAEP, IL5RA, PSPN, CLEC6A, ANXA10, CD70, TAFA5, LPO, CEACAM3, CAPG, DCN, NCAN, TSLP, CFC1, and AGR2. In further embodiments, the panel comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF. In other embodiments, the panel comprises CX3CL1, IDS, IL1B, NCAN, ODAM, PAEP, PSPN, RABGAP1L, SH2D1A, SMAD1, SMAD5, TNFRSF9 and WAS. These representative panels demonstrate that multiple distinct and minimally overlapping biomarker combinations drawn from a common candidate pool may achieve comparable predictive performance.

In certain embodiments, the machine learning classifier model is a logistic regression model. The machine learning classifier model may be trained using a feature selection process selected from the group consisting of least absolute shrinkage and selection operator (LASSO) regression, Random Forest, support vector machine-recursive feature elimination (SVM-RFE), and combinations thereof. In representative embodiments, the classifier model achieves an area under the receiver operating characteristic curve of 1.000 as demonstrated in a training cohort comprising GEP1 and GEP2 uveal melanoma samples. The machine learning classifier can be trained on either gene expression profile (GEP) classification or clinical metastatic outcome data.

Classifying the subject may comprise identifying the subject as having high metastatic risk when the metastatic risk score exceeds a predetermined probability threshold and identifying the subject as having low metastatic risk when the metastatic risk score is below the predetermined probability threshold. The predetermined probability threshold may be determined using Youden's index.

In certain embodiments, the subject has not undergone fine-needle aspiration biopsy of the uveal melanoma tumor. The methods disclosed herein provide metastatic risk classification for patients lacking tumor-derived molecular data. A substantial proportion of uveal melanoma patients, reported to exceed forty percent of diagnosed cases in certain cohorts, do not undergo tumor biopsy due to anatomic, clinical, or patient-specific factors. The disclosed methods enable GEP-equivalent molecular risk stratification in this patient population through a minimally invasive aqueous humor sampling procedure. Predicted high-risk classifications from aqueous humor-based profiling have been observed to correspond with tumor characteristics consistent with confirmed high-risk disease, including increased tumor volume relative to predicted low-risk classifications.

In certain embodiments, the method further comprises administering a treatment to the subject based on the classification, wherein the treatment is selected from the group consisting of surgery, radiation therapy, plaque brachytherapy, photocoagulation, chemotherapy, immunotherapy, thermotherapy, and combinations thereof.

In another aspect, the invention provides a method for generating a metastatic risk classifier for uveal melanoma. The method comprises obtaining aqueous humor samples from a training cohort of subjects having uveal melanoma, wherein the training cohort includes subjects with known gene expression profile classifications; quantifying protein expression levels in the aqueous humor samples; selecting a panel of protein biomarkers from the quantified protein expression levels by applying at least one feature selection method selected from the group consisting of LASSO regression, Random Forest, and support vector machine-recursive feature elimination, wherein the selected protein biomarkers are differentially expressed between low-risk and high-risk uveal melanoma tumors; and training a machine learning classifier model on the protein expression values of the selected panel, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, to generate a metastatic risk classifier. In certain embodiments, the selection step comprises applying each of the three feature selection methods and selecting protein biomarkers identified as informative by all three methods, thereby producing a minimal, non-redundant protein panel. The classifier model may be validated using five-fold stratified cross-validation.

In a further aspect, the invention provides a diagnostic kit for stratifying metastatic risk in a subject having uveal melanoma. The kit comprises a collection device configured to receive an aqueous humor sample from the subject; an assay reagent system, such as a proximity extension assay, Meso Scale Discovery (electrochemiluminescence), Luminex multiplex immunoassay, ELISA, mass spectrometry, single molecule array (Simoa), or aptamer-based assay (e.g., SomaScan), configured to quantify protein expression levels in the aqueous humor sample; a panel of detection reagents for protein biomarkers comprising at least three protein biomarkers selected from the combinatorial biomarker group disclosed herein; and a non-transitory computer-readable storage medium storing instructions which, when executed by a processor, cause the processor to receive protein expression values generated by the multiplex proximity extension assay reagent system, apply a machine learning classifier model trained on aqueous humor samples from subjects with known uveal melanoma metastatic risk classifications, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, and output the metastatic risk score for stratifying the subject as having high metastatic risk or low metastatic risk. In certain embodiments, the panel of detection reagents comprises detection reagents for the protein biomarkers PAEP, SMARCA2, FAM3B, KEL, SLITRK6, ANXA10, TNFRSF9, ADCYAP1R1, DECR1, GDF2, and PDCD1.

The methods, compositions, and diagnostic kits disclosed herein provide a non-invasive, reproducible, and clinically deployable platform for metastatic risk stratification in uveal melanoma. The combinatorial biomarker framework disclosed herein supports multiple functionally equivalent classifier configurations and accommodates assay compatibility with multiplex proteomic platforms suitable for clinical deployment. The methods extend molecular risk stratification to patients for whom tumor biopsy is not feasible, contraindicated, or declined, and provide a complementary tool for use alongside or in place of conventional biopsy-based gene expression profiling.

### DRAWINGS

FIGS. 1A-IC. Demographic and clinical characteristics. (A) Overview of study design, including sample collection, (B) patient demographics, and (C) heatmap of clinical characteristics. The patient cohort includes samples from 33 GEP1 (low risk) and 15 GEP2 (high-risk) UM patients, all used for subsequent proteomic analysis. Created in BioRender. Huang, E. (2026) https://biorender.com/t4bewne, accessed on 8 Feb. 2026.

FIG. 2. Estimated Concentration Range of Key Proteins. Boxplots depicting absolute protein concentrations (pg/mL, log 10 scale) grouped by median concentration range. Each panel represents a different concentration range, with individual proteins plotted within each group. Concentration variability within each protein is represented by the interquartile range, median, and whiskers (1.5×IQR), with outliers shown as individual points.

FIGS. 3A-3B. A) Estimated concentration of selected proteins by GEP-PRAME classification. (B) Estimated concentration of differentially expressed proteins by stage. Created in BioRender. Huang, E. (2026) https://biorender.com/t4bewne, accessed on 8 Feb. 2026.

FIGS. 4A-4D. Enrichment of inflammatory and tumor microenvironment signaling of aqueous humor proteins in advanced-stage uveal melanoma. (A) Canonical pathway analysis of aqueous humor proteomics comparing AJCC Stage I-II and Stage III-IV tumors reveals the enrichment of inflammatory and tumor microenvironment-related pathways. Bars indicate activation z-scores, dots represent −log (p-value), and circle size reflects the number of molecules per pathway. (B) Upstream regulator analysis predicts the activation of VEGFA and CCL2. Red bars represent activation z-scores, and dots indicate −log (p-value). (C) Estimated concentrations of aqueous humor VEGFA and CCL2 are compared between Stage III-IV and Stage I-II tumors. Concentrations are presented on a logarithmic scale. (D) Expression levels of selected VEGFA downstream targets (FLT1, FGF2, LAG3, MMP1, KDR, CXCL8, CXCL10, and PDCD1) are elevated in advanced-stage disease, consistent with the activation of angiogenic and immune-modulatory signaling networks. Estimated concentrations are shown on a logarithmic scale. Statistical significance is indicated as *p<0.05, **p<0.01, and ***p<0.001.

FIG. 5. Methodology, workflow, and sample collection. Created in BioRender. Huang, E. (2026) https://biorender.com/t4bewne, accessed on 8 Feb. 2026.

FIG. 6. Distribution of absolute concentrations (pg/mL) for key proteins across all aqueous humor samples.

FIGS. 7A-7D. Estimated concentration of 23 selected proteins by (A) eye color, (B) gender, (C) age group, and (D) tumor location. Created in Created in BioRender. Huang, E. (2026) https://biorender.com/t4 bewne, accessed on 8 Feb. 2026.

FIG. 8. Regression plots for 66 proteins.

FIGS. 9A-9B. Data simulation (n=100) for (A) VEGFA, p<0.0001, and (B) CXCL1, p=0.0001. Dashed lines indicate the prediction interval, and dots represent observed AQ values plotted against the fitted prediction line.

FIG. 10. Pre-treatment subclassification of the uveal melanoma patient cohort (n=128). Pie chart depicting the distribution of aqueous humor samples across three subgroups defined by the availability and result of tumor-derived gene expression profile (GEP) classification. GEP1 (low metastatic risk, blue) comprised 47 samples (36.7% of the cohort); GEP2 (high metastatic risk, red) comprised 28 samples (21.9% of the cohort); and GEP-unknown (GEP NA, gray), representing patients who did not undergo tumor fine-needle aspiration biopsy, comprised 53 samples (41.4% of the cohort). All samples were collected prior to any therapeutic intervention.

FIGS. 11A-11B. NPX expression of the 18 proteins comprising the Olink Flex-compatible classifier panel. (A) Boxplots depicting normalized protein expression (NPX, log 2 scale) for each of the 18 panel proteins in GEP1 (blue, n=47) and GEP2 (red, n=28) aqueous humor samples. Proteins are displayed along the x-axis. Individual data points represent outliers. Statistical significance between GEP1 and GEP2 groups is indicated above each protein: ns, not significant; *, p<0.05; **, p<0.01; ***, p<0.001; ****, p<0.0001. Proteins including TNFRSF9, GZMA, CXCL10, FLT3LG, PDCD1, KLRD1, and HBEGF demonstrated statistically significant higher NPX values in GEP2 samples relative to GEP1 samples. (B) Bar chart depicting the expression difference (delta NPX, calculated as GEP2 minus GEP1) for each of the 18 panel proteins. Positive values indicate higher expression in GEP2 samples. Statistical significance indicators correspond to those described in panel A. TNFRSF9 and HBEGF demonstrated the largest magnitude expression differences between GEP1 and GEP2 groups.

FIG. 12. Estimated absolute concentrations of 11 of the 18 Olink Flex-compatible panel proteins in GEP1 (blue) and GEP2 (red) aqueous humor samples. Protein concentrations (pg/mL) are presented on a logarithmic scale (y-axis, log 10). The 11 proteins depicted are CCL2, CD28, GZMA, TNFRSF9, CXCL10, IL17D, FLT3LG, CXCL8, IL6, FGF2, and PDCD1. Concentrations were derived from regression-based calibration models using paired qPCR-based proximity extension assay measurements as reference values. Boxplots depict median, interquartile range, and whiskers extending to 1.5 times the interquartile range, with outliers shown as individual points. Statistical significance between GEP1 and GEP2 groups is indicated above each protein: *, p<0.05; **, p<0.01; ***, p<0.001. GZMA, TNFRSF9, CXCL10, FLT3LG, and PDCD1 demonstrated statistically significant concentration differences between GEP1 and GEP2 groups. The majority of proteins in this subset exhibited median estimated concentrations above the 5 pg/mL detection threshold, supporting the feasibility of clinical assay development for this panel.

FIGS. 13A-13B. Performance of the 18-protein logistic regression classifier in GEP-classified aqueous humor samples (GEP1, n=47; GEP2, n=28). (A) Histogram depicting the predicted probability distribution for GEP1 (blue) and GEP2 (red) samples generated by the trained 18-protein logistic regression classifier. The x-axis represents the predicted probability of GEP2 classification, and the y-axis represents the frequency of samples at each probability value. The dashed vertical line at 0.50 indicates the classification threshold. GEP1 samples are concentrated at predicted probabilities below 0.1, while GEP2 samples are concentrated at predicted probabilities above 0.5, demonstrating clear separation between risk groups. (B) Receiver operating characteristic (ROC) curve for the 18-protein classifier applied to GEP-classified samples. The x-axis represents the false positive rate and the y-axis represents the true positive rate. The dashed diagonal line represents chance-level performance. The classifier achieved an area under the curve (AUC) of 1.000, indicating complete discrimination between GEP1 and GEP2 samples in the training cohort.

FIGS. 14A-14B. Application of the 18-protein logistic regression classifier to GEP-unknown aqueous humor samples (n=53). (A) Histogram depicting the predicted probability distribution for GEP-unknown samples. The x-axis represents the predicted probability of GEP2 classification generated by the trained classifier, and the y-axis represents the frequency of samples at each probability value. The distribution demonstrates a bimodal pattern, with the majority of samples receiving predicted probabilities below 0.1 and a subset receiving predicted probabilities approaching 1.0, consistent with the distribution pattern observed in GEP-classified samples. (B) Histogram depicting the protein expression profile (PEP) classification of GEP-unknown samples following application of the 0.50 probability threshold. Samples with predicted probabilities below 0.50 were classified as PEP1 (potential low metastatic risk, light gray, n=41) and samples with predicted probabilities at or above 0.50 were classified as PEP2 (potential high metastatic risk, dark gray, n=12). The dashed vertical line indicates the 0.50 classification threshold. PEP2-classified samples exhibited larger mean tumor volumes compared to PEP1-classified samples (356.5±385.6 mm³ versus 85.18±100.3 mm³; p=0.013), consistent with the tumor burden characteristics associated with confirmed GEP2 classification in the training cohort.

FIG. 15. Depicts a greedy selection.

FIG. 16. Provides further characteristics of the biomarkers disclosed herein.

### DESCRIPTION

Accurate stratification of metastatic risk at the time of diagnosis is needed for tailoring surveillance strategies, guiding therapeutic decisions, and informing patient management. The current clinical standard for metastatic risk stratification in uveal melanoma relies on gene expression profiling (GEP) of tumor tissue obtained via fine-needle aspiration biopsy (FNAB). GEP classifies tumors into low-risk (GEP1) and high-risk (GEP2) categories based on transcriptional signatures. Additional molecular markers, such as Preferentially Expressed Antigen in Melanoma (PRAME) status and BRCA1-associated protein 1 (BAP1) mutation status, provide complementary prognostic information. However, tumor biopsy-based profiling presents several limitations. FNAB is an invasive procedure associated with risks including retinal detachment, intraocular hemorrhage, vision loss, and tumor seeding. Biopsy may be contraindicated or technically unfeasible due to tumor size, anatomic location, or patient-specific clinical factors. In real-world practice, a substantial proportion of uveal melanoma patients—reported to exceed thirty percent in some cohorts—do not undergo tumor biopsy for these reasons or due to patient preference. Furthermore, biopsy samples are subject to sampling variability, and repeat biopsies to monitor tumor evolution are generally not feasible.

Liquid biopsy approaches have emerged as alternatives for molecular profiling across oncology applications. In uveal melanoma, the aqueous humor (AH) of the anterior chamber of the eye has been investigated as a biofluid for molecular analysis. Aqueous humor can be obtained through clear corneal paracentesis, a procedure with a lower risk profile than tumor FNAB and reflects the intraocular tumor microenvironment. Prior proteomic studies have identified molecular subtypes of uveal melanoma through analysis of aqueous humor and have demonstrated that distinct immunological and stromal signaling signatures are detectable in this biofluid.

Prior aqueous humor-based approaches, however, have presented limitations that have constrained clinical translation. Earlier studies have frequently relied on targeted cytokine panels or hypothesis-driven biomarker selection, which restrict the biomarker discovery space. Relative quantification metrics, such as normalized protein expression (NPX) values, have been used in discovery-phase studies but do not establish whether protein signals are present at concentrations compatible with standard clinical immunoassays, limiting reproducibility across cohorts and hindering the development of diagnostic thresholds. Prior studies have characterized molecular variation in aqueous humor but have not produced patient-level predictive models validated against GEP-based risk classification, nor have they addressed the clinical scenario in which risk stratification is needed for patients lacking tumor-derived molecular data. Additionally, classifier models described in the literature have generally been limited to fixed biomarker panels, without establishing a framework permitting selection of alternative protein subsets suitable for different assay platforms or clinical deployment contexts.

A need therefore exists for non-invasive, reproducible, and clinically deployable methods for stratifying metastatic risk in uveal melanoma patients, including those for whom tumor biopsy is not feasible or has not been performed. A need further exists for biomarker frameworks that accommodate multiple functionally equivalent protein panels, support calibrated quantification at clinically measurable concentrations, and integrate with machine learning-based classifiers to provide patient-level risk predictions. The present invention addresses these needs. Further, as longitudinal clinical outcome data become available, the disclosed biomarker framework is applicable to direct prediction of metastatic outcomes, including metastasis-free survival, time to metastasis, or occurrence of metastasis.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “of” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the specified value. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term “about” can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in a standard or control sample or another patient sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels that correspond to, for example, a patient UM.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has or at risk of having metastatic cancer.

The terms “patient,” “individual,” or “subject” are used interchangeably herein, and refer to a mammal, particularly, a human. As used herein, a “subject in need thereof” is a patient, animal, mammal, or human, who will benefit from the method of this invention. The patient may have mild, intermediate or severe disease, or may only experience transient, or fluctuating symptoms. The patient may be an individual, at risk of developing a disease, in need of treatment or in need of diagnosis based on particular symptoms or family history. In some cases, the terms may refer to treatment of animals, in such as veterinary applications (e.g., companion animals (e.g., cats or dogs) or livestock (e.g., pigs, cows, horses)).

The terms “measuring” and “determining” are used interchangeably throughout and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting level of one or more biomarkers in a patient sample. Measuring can be accomplished by methods known in the art and those further described herein including. The term “measuring” is also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms. Moreover, a sample obtained from a patient can be divided and only a portion may be used for diagnosis. Further, the sample, or a portion thereof, can be stored under conditions to maintain sample for later analysis.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed in a sample from an unaffected individual (UI) or a normal control individual (NC) (both terms are used interchangeably herein) or other affected individual. In another embodiment, a “suitable control” or “appropriate control” is a value, level, feature, characteristic, property, etc. determined prior to performing a therapy on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc.

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, “uveal melanoma (UM)” is defined as a primary intraocular malignancy arising from melanocytes of the uveal tract, comprising the choroid, ciliary body, and iris. As used herein, uveal melanoma refers to all anatomical subtypes of uveal tract melanoma, including choroidal melanoma, ciliary body melanoma, and iris melanoma.

As used herein, “gene expression profile (GEP)” is a tumor-derived molecular classification system applied to uveal melanoma that categorizes tumors based on transcriptional signatures obtained from fine-needle aspiration biopsy specimens. As used herein, GEP classification is performed by analysis of tumor tissue and assigns tumors to one of two risk categories: GEP1 (low metastatic risk) or GEP2 (high metastatic risk). Gene expression profile (GEP) classification is a clinically validated surrogate marker associated with metastatic risk in uveal melanoma.

As used herein, “GEP1” is a gene expression profile classification designating uveal melanoma tumors with low metastatic risk, associated with a lower probability of systemic metastasis, particularly to the liver.

As used herein, “GEP2” is a gene expression profile classification designating uveal melanoma tumors with high metastatic risk, associated with a higher probability of systemic metastasis, particularly to the liver. Further, “GEP2” is associated with elevated metastatic risk (e.g., within 5 years), based on established clinical data.

As used herein, “GEP-unknown (GEP NA)” is a designation applied to uveal melanoma patients for whom tumor-derived gene expression profile data is unavailable, including patients who have not undergone fine-needle aspiration biopsy, patients for whom biopsy was performed but did not yield sufficient material for GEP analysis, and patients for whom GEP results are inconclusive or discordant.

As used herein, machine learning classifier is trained on aqueous humor protein expression values from a training cohort of subjects having uveal melanoma, and on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma.

As used herein, “protein expression profile (PEP) is a risk classification generated by application of the aqueous humor-based proteomic classifier described herein to a subject lacking tumor-derived GEP data. As used herein, PEP1 designates a predicted low metastatic risk classification and PEP2 designates a predicted high metastatic risk classification, corresponding to GEP1 and GEP2 respectively.

As used herein, “PEP1” is a protein expression profile classification generated by the aqueous humor-based classifier described herein, designating subjects predicted to have low metastatic risk, corresponding to the GEP1 molecular phenotype.

As used herein, “PEP2” is a protein expression profile classification generated by the aqueous humor-based classifier described herein, designating subjects predicted to have high metastatic risk, corresponding to the GEP2 molecular phenotype.

As used herein, “pre-treatment subclassification” is the assignment of a uveal melanoma patient to a molecular risk category based on data obtained prior to initiation of any therapeutic intervention for uveal melanoma, including prior to plaque brachytherapy, proton beam radiation, enucleation, chemotherapy, immunotherapy, or other treatment modalities.

As used herein, “proximity extension assay (PEA)” is a multiplexed immunoassay technology in which pairs of antibodies conjugated to complementary oligonucleotide probes bind to target proteins in proximity, enabling extension of a double-stranded DNA reporter that is subsequently quantified. As used herein, proximity extension assay includes both next generation sequencing-based PEA and qPCR-based PEA platforms, including without limitation the Olink Explorer and Olink Flex platforms. The proximity extension assay is one non-limiting embodiment. The protein biomarkers may be quantified by any suitable detection platform, including, but not limited to, proximity extension assay (PEA), Meso Scale Discovery (electrochemiluminescence), Luminex multiplex immunoassay, ELISA, mass spectrometry, single molecule array (Simoa) and/or aptamer-based assay (e.g., SomaScan).

As used herein, “normalized protein expression (NPX)” is a unitless, log 2-transformed relative protein abundance value generated by the Olink proximity extension assay analytical pipeline. NPX values reflect relative protein abundance between samples within the same analytical run and are not directly equivalent to absolute protein concentrations. As used herein, NPX values are used as inputs to the machine learning classifier model.

As used herein, “Olink Flex” is a targeted proximity extension assay platform enabling multiplexed quantification of a user-defined subset of proteins selected from the Olink protein library. As used herein, the Olink Flex platform is used to generate NPX values for the 18-protein classifier panel described herein, and proteins described as Flex-compatible or Flex-available are proteins for which validated Olink Flex assays exist.

As used herein, “absolute quantification (AQ)” is a measurement of protein concentration expressed in units of mass per volume, specifically picograms per milliliter (pg/mL), as distinguished from relative expression metrics such as NPX values. As used herein, absolute quantification values are derived from regression-based calibration models using qPCR-based PEA measurements as reference concentration anchors.

As used herein, “estimated concentration” is a regression-modeled protein concentration value expressed in picograms per milliliter, derived by applying a linear regression model relating NPX values to experimentally measured absolute concentration values obtained from qPCR-based PEA analysis of a subset of samples. As used herein, estimated concentrations represent calibrated modeled values rather than direct measurements across the full cohort.

As used herein, “detection threshold is a minimum protein concentration value, expressed in picograms per milliliter, below which reliable quantification using a given assay platform is not achievable under standard conditions. As used herein, a detection threshold of about 5 pg/mL is applied as a criterion for assessing the feasibility of clinical assay development for individual proteins.

As used herein, “delta NPX” is the difference in mean or median normalized protein expression value between two comparison groups, calculated as the NPX value of the GEP2 group minus the NPX value of the GEP1 group for a given protein. A positive delta NPX value indicates higher expression in GEP2 samples relative to GEP1 samples.

As used herein, “logistic regression model” is a statistical classification model that estimates the probability of a binary outcome as a function of one or more predictor variables using a logistic function. As used herein, the logistic regression model receives NPX values for the proteins in the classifier panel as inputs and outputs a predicted probability of GEP2 classification for each sample.

As used herein, “logit(p): is the log-odds transformation of a predicted probability p, defined as the natural logarithm of p divided by (1 minus p), used as the linear combination of weighted predictor variables in the logistic regression model described herein.

As used herein, “predicted probability it the output of the logistic regression classifier representing the estimated probability that a given aqueous humor sample is derived from a GEP2 (high metastatic risk) uveal melanoma, expressed as a value between 0 and 1.

As used herein, “predicted probability distribution” is the frequency distribution of predicted probability values across a cohort of samples, used to assess the separation between GEP1 and GEP2 classified samples or between PEP1 and PEP2 classified samples following application of the classifier.

As used herein, “classification threshold” is a predetermined predicted probability value above which a sample is classified as GEP2 or PEP2 (high metastatic risk) and at or below which a sample is classified as GEP1 or PEP1 (low metastatic risk). As used herein, the classification threshold is 0.50 unless otherwise specified. In certain embodiments, the classification threshold is determined using Youden's index.

As used herein, “receiver operating characteristic (ROC) curve” is a graphical representation of classifier performance depicting the true positive rate (sensitivity) on the y-axis against the false positive rate (1 minus specificity) on the x-axis across all possible classification thresholds. As used herein, ROC curve analysis is used to evaluate the discriminative performance of the classifier between GEP1 and GEP2 samples.

As used herein, “area under the curve (AUC)” is the area under the receiver operating characteristic curve, representing the probability that the classifier assigns a higher predicted probability to a randomly selected GEP2 sample than to a randomly selected GEP1 sample. AUC values range from 0.5 (chance-level performance) to 1.0 (perfect discrimination). As used herein, an AUC of 1.000 indicates complete separation of GEP1 and GEP2 samples by the classifier in the training cohort.

As used herein, “true positive rate” is the proportion of GEP2 samples correctly classified as GEP2 by the classifier at a given classification threshold, equivalent to sensitivity.

As used herein, “False positive rate” is the proportion of GEP1 samples incorrectly classified as GEP2 by the classifier at a given classification threshold, equivalent to 1 minus specificity.

As used herein, “LASSO regression” describes the Least Absolute Shrinkage and Selection Operator regression, a regularized linear regression method that applies an L1 penalty to model coefficients, shrinking non-informative coefficients toward zero and thereby performing feature selection by retaining only proteins with non-zero coefficients. As used herein, LASSO regression with 10-fold cross-validation is applied as one of three feature selection methods used to identify proteins predictive of GEP classification.

As used herein, “Random Forest”: is an ensemble machine learning method comprising a collection of decision trees trained on bootstrap samples of the training data, with feature importance assessed based on mean decrease in impurity across all trees. As used herein, Random Forest with 1,000 estimators is applied as one of three feature selection methods.

As used herein, “Support vector machine-recursive feature elimination (SVM-RFE)” is a feature selection method in which a support vector machine classifier is trained iteratively, with the least informative features removed at each iteration based on their contribution to the classification boundary, until a desired number of features is retained. As used herein, SVM-RFE is applied as one of three feature selection methods.

As used herein, “Five-fold stratified cross-validation” is a model validation procedure in which the dataset is partitioned into five subsets of approximately equal size, with each subset used once as a held-out validation set while the remaining four subsets are used for model training, and with stratification applied to maintain the proportion of GEP1 and GEP2 samples across all folds. As used herein, five-fold stratified cross-validation is used to assess classifier performance and estimate generalizability.

As used herein, “aqueous humor” is a clear, protein-containing fluid occupying the anterior and posterior chambers of the eye, produced by the ciliary body epithelium and drained through the trabecular meshwork. As used herein, aqueous humor refers to fluid collected from the anterior chamber of the eye by clear corneal paracentesis and used as the biological sample for proteomic profiling.

As used herein, “fine-needle aspiration biopsy (FNAB)” is an intraocular biopsy procedure in which a fine-gauge needle is inserted into the eye to aspirate tumor cells or tissue from a uveal melanoma for molecular analysis, including gene expression profiling and PRAME status assessment. As used herein, fine-needle aspiration biopsy is the procedure from which tumor-derived GEP data is obtained in clinical practice.

As used herein, “AJCC stage” describes the clinical stage of uveal melanoma as defined by the American Joint Committee on Cancer staging system, based on tumor size, extent of ciliary body involvement, extraocular extension, regional lymph node involvement, and distant metastasis. As used herein, AJCC stages I through IV are used to characterize disease extent, with Stages I and II representing localized disease and Stages III and IV representing locally advanced or metastatic disease.

As used herein, “tumor volume” is a quantitative measure of uveal melanoma tumor size calculated from clinical imaging or ultrasonographic measurements, expressed in cubic millimeters. As used herein, tumor volume is used as a clinical correlate for validating the biological relevance of protein expression profile classifications in GEP-unknown samples.

As used herein, “tumor location” is the anatomical position of the uveal melanoma primary tumor within the eye, classified as anterior (involving the iris or ciliary body) or posterior (involving the choroid posterior to the equator). As used herein, tumor location is assessed as a clinical covariate in cohort characterization.

As used herein, “PRAME” describes Preferentially Expressed Antigen in Melanoma, a cancer-testis antigen whose expression status in uveal melanoma tumor tissue is assessed as an independent prognostic biomarker for metastatic risk, with PRAME-positive status associated with increased metastatic risk within both GEP1 and GEP2 classification groups.

The following proteins comprising the 18-protein classifier panel require identification by full name and functional context for use in the specification:

As used herein, “TNFRSF9” is Tumor Necrosis Factor Receptor Superfamily Member 9, also designated 4-1BB or CD137, a co-stimulatory receptor expressed on activated T cells and natural killer cells involved in immune activation and T cell survival signaling.

As used herein, “CD70” is Cluster of Differentiation 70, also designated TNFSF7 or CD27 ligand, a member of the tumor necrosis factor superfamily expressed on activated T cells, B cells, and dendritic cells, functioning as a ligand for CD27 and involved in lymphocyte activation and survival.

As used herein, “TNFRSF13C” is Tumor Necrosis Factor Receptor Superfamily Member 13C, also designated BAFF-R, a receptor for B cell activating factor involved in B cell survival, maturation, and homeostasis.

As used herein, “PROK1” is Prokineticin 1, also designated EG-VEGF, a secreted protein involved in angiogenesis, immune cell recruitment, and regulation of the tumor microenvironment.

As used herein, “CEACAM21” is Carcinoembryonic Antigen-Related Cell Adhesion Molecule 21, a member of the carcinoembryonic antigen family involved in cell adhesion and immune regulation.

As used herein, “PDCD1” is Programmed Cell Death Protein 1, also designated PD-1 or CD279, an immune checkpoint receptor expressed on activated T cells, B cells, and natural killer cells that negatively regulates immune responses upon binding to its ligands PD-L1 and PD-L2.

As used herein, “GZMA” is Granzyme A, a serine protease expressed by cytotoxic T lymphocytes and natural killer cells involved in target cell apoptosis and immune-mediated tumor cell killing.

As used herein, “CCL2” is C-C Motif Chemokine Ligand 2, also designated monocyte chemoattractant protein-1 (MCP-1), a chemokine that recruits monocytes, memory T cells, and dendritic cells to sites of inflammation and tumor tissue.

As used herein, “IL6” is Interleukin-6, a pleiotropic cytokine involved in acute phase responses, immune cell differentiation, and tumor-associated inflammatory signaling.

As used herein, “FGF2” is Fibroblast Growth Factor 2, also designated basic FGF (bFGF), a growth factor involved in angiogenesis, cell proliferation, and tumor microenvironment remodeling.

As used herein, “CXCL10” is C-X-C Motif Chemokine Ligand 10, also designated interferon gamma-induced protein 10 (IP-10), a chemokine involved in recruitment of activated T cells and natural killer cells to sites of inflammation and tumor tissue.

As used herein, “CXCL8” is C-X-C Motif Chemokine Ligand 8, also designated interleukin-8 (IL-8), a pro-inflammatory chemokine involved in neutrophil recruitment, angiogenesis, and tumor progression.

As used herein, “IL17D” is Interleukin-17D, a member of the IL-17 cytokine family involved in inflammatory signaling and immune regulation.

As used herein, “KLRD1” is Killer Cell Lectin-Like Receptor D1, also designated CD94, a C-type lectin receptor expressed on natural killer cells and cytotoxic T lymphocytes involved in regulation of cytotoxic immune responses.

As used herein, “CD28” is Cluster of Differentiation 28, a co-stimulatory receptor expressed on T cells that provides activating signals required for full T cell activation upon binding to CD80 or CD86 on antigen-presenting cells.

As used herein, “CD79B” is Cluster of Differentiation 79B, also designated immunoglobulin-associated beta (Igbeta), a component of the B cell receptor complex involved in B cell activation and signaling.

As used herein, “FLT3LG” is FMS-Like Tyrosine Kinase 3 Ligand, a cytokine that promotes the differentiation and expansion of hematopoietic progenitor cells, dendritic cells, and natural killer cells.

As used herein, “HBEGF” is Heparin-Binding Epidermal Growth Factor-Like Growth Factor, a member of the epidermal growth factor family involved in cell proliferation, angiogenesis, and tumor microenvironment signaling through binding to EGFR and ErbB4 receptors.

Methods involving conventional molecular biology techniques are described herein. Such techniques are generally known in the art and are described in detail in methodology treatises, such as Molecular Cloning: A Laboratory Manual, Sambrook et al., Cold Spring Harbor Laboratory Press, Cold Spring Harbor, N.Y.

As used herein, reference to a protein by its gene symbol or UniProt accession identifier refers to the full-length mature human protein corresponding to that gene symbol or identifier as deposited in the UniProt database, including all naturally occurring isoforms and post-translationally modified forms thereof. For example, reference to TNFRSF9 refers to the protein deposited under UniProt accession Q07011; reference to CD70 refers to the protein deposited under UniProt accession P32970; reference to PDCD1 refers to the protein deposited under UniProt accession Q15116; and so forth for each protein biomarker recited herein. The methods and compositions disclosed herein encompass detection and quantification of these proteins and any fragments, isoforms, or modified forms thereof that retain antigenic reactivity with antibodies or detection reagents specific to the reference protein.

### Uses

Uveal melanoma carries a substantial risk of systemic metastasis, primarily to the liver, yet accurate identification of patients at high metastatic risk at the time of diagnosis depends on molecular profiling of tumor tissue obtained by fine-needle aspiration biopsy of the intraocular tumor. Gene expression profiling of such biopsy material provides the current clinical standard for risk stratification, classifying tumors as GEP1 (low metastatic risk) or GEP2 (high metastatic risk). However, this approach is constrained by the invasive nature of intraocular biopsy, which carries procedural risks including hemorrhage, retinal detachment, and tumor seeding, and is not performed in a substantial proportion of patients due to anatomic, clinical, or patient-related factors. As demonstrated in the cohort described herein, wherein 53 of 128 uveal melanoma patients (41.4%) lacked tumor-derived GEP data at the time of diagnosis, the inability to perform or obtain biopsy results in a large fraction of patients receiving no molecular risk classification, leaving clinicians without the prognostic information needed to direct surveillance intensity, inform treatment planning, or identify candidates for systemic therapy. This gap in molecular risk assessment represents a barrier to individualized patient management and disproportionately affects patients with tumors of small size, anteriorly located tumors, patients with medical contraindications to biopsy, and patients who decline the procedure.

The present invention addresses this problem by providing a non-invasive, aqueous humor-based proteomic classifier that generates a molecular risk classification equivalent to tumor-derived GEP results without requiring intraocular tumor biopsy. Aqueous humor, collected by clear corneal paracentesis at the time of clinically indicated surgery, contains measurable levels of proteins that reflect the biology of the intraocular tumor microenvironment and that differ systematically between GEP1 and GEP2 tumors. Through assay-based proteomic profiling and machine learning-based feature selection, the inventors identified protein panels, including an 18-protein classifier comprising TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF (and others as disclosed herein), that discriminate GEP1 from GEP2 tumors with an area under the receiver operating characteristic curve of 1.000 in the training cohort of 75 GEP-classified samples. Application of this classifier to the 53 GEP-unknown samples yielded protein expression profile classifications of PEP1 (n=41) and PEP2 (n=12), with PEP2-classified patients exhibiting larger mean tumor volumes (356.5±385.6 mm³) compared to PEP1-classified patients (85.18±100.3 mm³; p=0.013), consistent with the tumor burden characteristics associated with confirmed GEP2 classification, thereby providing clinical validation of the classifier output in the absence of tumor biopsy data. The disclosed methods thus extend molecular risk stratification to the substantial proportion of uveal melanoma patients who would otherwise receive no prognostic molecular classification, enabling individualized surveillance and treatment planning across the full clinical population.

The methods, compositions, and kits described herein have applications across multiple stages of uveal melanoma diagnosis, prognosis, treatment planning, and disease monitoring. In one application, the disclosed methods provide non-invasive stratification of uveal melanoma into GEP1 (low metastatic risk) and GEP2 (high metastatic risk) categories through proteomic profiling of aqueous humor, yielding a molecular risk classification that corresponds to tumor-derived gene expression profile results without requiring intraocular tumor biopsy.

In a related application, the methods provide liquid biopsy-based risk assessment for patients in whom traditional tumor fine-needle aspiration biopsy is contraindicated, unfeasible, or declined, including patients with tumors of small size, tumors in anatomically challenging locations, patients with medical comorbidities that preclude fine-needle aspiration biopsy, and patients who elect not to undergo tumor biopsy due to procedural risk or personal preference, thereby extending molecular risk stratification to the substantial proportion of uveal melanoma patients who would otherwise lack tumor-derived prognostic data.

The methods further support real-time or longitudinal monitoring of metastatic risk through repeat aqueous humor sampling over the course of disease management, enabling detection of dynamic changes in biomarker expression that may reflect tumor evolution, treatment response, emerging resistance, or progression toward metastatic disease, and supporting the development of patient-specific surveillance schedules with adjustment of monitoring intensity based on evolving molecular risk profiles.

The disclosed biomarker panels and classifiers also provide complementary input for immunotherapy decision-making, including identification of patients likely to benefit from immune checkpoint inhibitors, adoptive cell therapies, tumor-infiltrating lymphocyte therapies, bispecific T-cell engagers, and other immunomodulatory treatments, as the panels include immune-related proteins such as PDCD1, TNFRSF9, CD28, CD70, GZMA, and CCL2, the expression of which can inform selection of immunotherapeutic regimens and prediction of therapeutic response.

In further embodiments, the methods support risk-adapted surveillance planning, wherein subjects classified as high metastatic risk are directed to more frequent systemic imaging, hepatic monitoring, and laboratory assessments, while subjects classified as low metastatic risk undergo less intensive surveillance protocols.

The methods additionally provide adjunctive diagnostic input in cases where tumor biopsy yields inconclusive, insufficient, or discordant gene expression profile results, providing an orthogonal molecular assessment to support clinical decision-making. In additional embodiments, the methods support selection of patients for enrollment in clinical trials evaluating therapies for high-risk or metastatic uveal melanoma, guidance of treatment intensification or de-escalation decisions in the adjuvant setting following local tumor control through plaque brachytherapy, proton beam radiation, or enucleation, and integration with additional liquid biopsy modalities, including circulating tumor DNA, circulating tumor cells, and plasma proteomic analyses, to provide a multi-analyte assessment of tumor burden and metastatic potential.

The methods further support development and validation of companion diagnostic assays for therapeutic agents directed against targets identified within the biomarker panels, including agents targeting VEGFA-associated signaling pathways, immune checkpoint pathways, and chemokine signaling axes implicated in uveal melanoma progression, as well as post-treatment assessment of residual molecular risk, identification of candidates for adjuvant systemic therapy, and support of clinical decision-making regarding globe-sparing versus enucleation procedures by providing additional prognostic information at the time of diagnosis.

In yet further embodiments, the methods support population-level research applications, including epidemiologic studies of uveal melanoma biology and comparative effectiveness research evaluating outcomes across molecular risk strata, and training and refinement of machine learning classifier models through incorporation of expanded aqueous humor proteomic datasets to further improve predictive accuracy and generalizability across diverse patient populations.

### EXEMPLARY EMBODIMENTS

In certain embodiments, the present invention provides biomarker panels and combinatorial biomarker frameworks for predicting metastatic risk in uveal melanoma using protein expression measured in aqueous humor (AH). The disclosed methods enable non-invasive risk stratification and are particularly applicable to patients who do not undergo tumor biopsy or who lack tumor-derived molecular data. The invention further provides a framework in which multiple distinct biomarker subsets may be derived from a shared candidate pool while maintaining predictive performance.

In certain embodiments, aqueous humor is utilized as a liquid biopsy medium that reflects tumor-associated biological processes. Proteins present in aqueous humor may originate from tumor cells, immune cells within the tumor microenvironment, stromal and vascular components, as well as extracellular vesicles and secreted proteins. As a result, aqueous humor proteomic profiles capture an integrated representation of tumor biology, including immune regulation, angiogenesis, and tumor progression, thereby enabling surrogate assessment of tumor behavior without direct tumor sampling.

In certain embodiments, the biomarker panel comprises one or more proteins selected from the group consisting of: ACP5, ADAM22, ADCYAP1R1, ADH4, AGXT, AHSP, AIF1, AMBP, ANXA10, ANXA4, APLP1, ARG1, ATF2, AXIN1, BANK1, BIRC2, BRK1, CA1, CALCOCO1, CAPG, CASP1, CASP10, CASP2, CCL2, CCL3, CCL4, CCL7, CCL8, CCL11, CCL17, CCL19, CCL24, CCL25, CD27, CD28, CD33, CD6, CD70, CD74, CD79B, CDNF, CEACAM3, CEACAM21, CEBPB, CEP164, CES3, CLEC4A, CLEC6A, CLIP2, CLSPN, CNTN3, CRHR1, CRX, CSF1, CSF3, CXCL8, CXCL9, CXCL10, CXCL11, CXCL13, DAPP1, DCN, DCTN1, DECR1, DGKZ, DNAJA2, DNAJB1, DPT, F9, FAM3B, FASLG, FCER2, FCGR2B, FCRL3, FES, FGF2, FGF5, FKBPlB, FKBP7, FLT1, FLT3LG, FLT4, FMR1, FRZB, GDF2, GGA1, GNLY, GP2, GUSB, GZMA, HBEGF, HEBP1, HGF, HMBS, ICAM4, IFNG, IFNLR1, IGSF3, IKZF2, IL2RA, IL4R, IL5RA, IL6, IL15, IL17D, IL18, IL18RAP, IL19, IL1RN, IL32, IL33, IL34, ING1, IRAK1, ITGA6, ITGB6, ITGB7, IVD, KDR, KEL, KIR2DL3, KLB, KLK1, KLK12, KLRD1, KRT19, LAG3, LAIR2, LAP3, LBR, LEP, LPO, LSM1, MAPT, METAP1, MFAP3, MGMT, MITD1, MMP1, MMP12, MRPL46, MSMB, MYO9B, MZB1, NCAN, NINJ1, NMNAT1, NPM1, NPPB, NTF3, NUDC, ODAM, OGFR, OLR1, OMD, OSM, PAEP, PDCD1, PDCD6, PDGFC, PGF, PFDN2, PKLR, PMVK, PQBP1, PRCP, PROK1, PRTFDC1, PSRC1, PSPN, PTPRM, RAB6B, RASA1, RCOR1, RRM2, SCGN, SERPINB9, SFTPA1, SIRT5, SIT1, SLAMF1, SLAMF6, SLITRK6, SMAD5, SMARCA2, SRP14, STIP1, STX16, SUGT1, SUSD1, TAFA5, TARBP2, TBC1D23, TBCB, TCL1B, TIA1, TNF, TNFRSF4, TNFRSF9, TNFRSF11A, TNFRSF13C, TNFSF11, TNFSF12, TNNI3, TPPP3, TSLP, TXLNA, UBAC1, UXS1, VEGFA, VIM, VSTM2L, VTCN1, VWF, WARS, WASF1, XRCC4, YTHDF3, ZBTB17, CFC1, AGR2, CX3CL1, IDS, IL1B, RABGAPIL, SH2D1A, SMAD1 and/or WAS.

The biomarker group defines a comprehensive aqueous humor proteomic feature space from which multiple distinct subsets may be selected. The biomarker list is cumulative and non-limiting, and biomarkers can be used individually or in combination. Different subsets derived from the biomarker group can yield equivalent predictive performance, reflecting the distributed nature of biological signals captured in aqueous humor.

In certain embodiments, the disclosed biomarkers define a combinatorial biomarker space in which multiple distinct biomarker subsets can be constructed. Such subsets can be overlapping or non-overlapping and can share minimal or no common proteins. Despite differences in composition, distinct biomarker panels can achieve equivalent classification performance, demonstrating that predictive signal is not dependent on any single biomarker but is instead distributed across multiple proteins.

In certain embodiments, a first biomarker panel comprising PAEP, IL5RA, PSPN, CLEC6A, ANXA10, CD70, TAFA5, LPO, CEACAM3, CAPG, DCN, NCAN, TSLP, CFC1, and AGR2 provides predictive classification of metastatic risk. In certain embodiments, a second biomarker panel comprising TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF provides equivalent predictive performance despite minimal overlap with the first panel. In certain embodiments, a third biomarker panel comprising PAEP, TBCB, PDCD1, SMARCA2, ADCYAP1R1, TNFRSF9, FAM3B, SLITRK6, ZBTB17, KEL, GDF2, ANXA10, SLAMF1, and DECR1 represents an additional alternative configuration. In certain embodiments, a fourth biomarker panel comprising CX3CL1, IDS, IL1B, NCAN, ODAM, PAEP, PSPN, RABGAP1L, SH2D1A, SMAD1, SMAD5, TNFRSF9 and WAS represents an additional alternative configuration. These embodiments demonstrate that multiple distinct biomarker combinations derived from the same biomarker group can achieve comparable classification accuracy.

Biomarker selection may be based on differential expression, detectability thresholds in aqueous humor, cross-cohort reproducibility, compatibility with various assays, and machine learning-based feature selection approaches. Protein biomarkers may be measured using proximity extension assays, enzyme-linked immunosorbent assays, mass spectrometry, or other multiplex immunoassay platforms (assays can include, but are not limited to, proximity extension assays, enzyme-linked immunosorbent assays, mass spectrometry, or other multiplex immunoassay platforms, Meso Scale Discovery (electrochemiluminescence), Luminex multiplex immunoassay, ELISA, mass spectrometry, single molecule array (Simoa), or aptamer-based assays (e.g., SomaScan)), and measurements can be normalized or transformed prior to analysis. Biomarker measurements can be input into predictive models including logistic regression, random forest, support vector machine, neural network, or other statistical or machine learning methods to generate a metastatic risk score.

In certain embodiments, biomarker panels can comprise a minimal subset of at least three biomarkers selected from the biomarker group while maintaining predictive performance. In certain embodiments, aqueous humor samples can be collected longitudinally to monitor disease progression or treatment response. The disclosed methods enable risk prediction without tumor biopsy and are applicable to patients lacking tumor-derived molecular data.

In certain embodiments, biomarkers can be replaced by other proteins that perform similar biological functions, participate in the same pathway, or provide comparable predictive performance, and such substitutions are considered within the scope of the invention.

Experimental support, such as that provided in the Examples below, includes multi-cohort aqueous humor proteomic datasets, identification of informative biomarkers through statistical and machine learning approaches, development of multiple classifier panels achieving high predictive performance, and validation across both GEP-classified and GEP-unknown cohorts with concordant risk predictions.

In certain embodiments, the invention provides a method for stratifying metastatic risk in a subject having uveal melanoma, comprising obtaining an aqueous humor sample from the subject, quantifying protein expression levels in the aqueous humor sample using an assay to quantify the protein(s), applying a machine learning classifier model to the protein expression values of a panel of protein biomarkers to generate a metastatic risk score, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, and classifying the subject as having high metastatic risk or low metastatic risk based on the metastatic risk score.

In certain embodiments, the aqueous humor sample is collected from the subject prior to any therapeutic intervention for uveal melanoma. In certain embodiments, the aqueous humor sample is obtained by clear corneal paracentesis. In certain embodiments, the subject has not undergone fine-needle aspiration biopsy of the uveal melanoma tumor. In certain embodiments, the subject lacks tumor-derived gene expression profile data at the time of sample collection.

In certain embodiments, the panel of protein biomarkers comprises one or more proteins selected from the full biomarker group consisting of ACP5, ADAM22, ADCYAP1R1, ADH4, AGXT, AHSP, AIF1, AMBP, ANXA10, ANXA4, APLP1, ARG1, ATF2, AXIN1, BANK1, BIRC2, BRK1, CA1, CALCOCO1, CAPG, CASP1, CASP10, CASP2, CCL2, CCL8, CCL24, CCL25, CD27, CD28, CD33, CD6, CD70, CD74, CD79B, CDNF, CEACAM3, CEACAM21, CEBPB, CEP164, CES3, CFC1, CLEC4A, CLEC6A, CLIP2, CLSPN, CNTN3, CRHR1, CRX, CSF3, CXCL8, CXCL10, CXCL13, DAPP1, DCN, DCTN1, DECR1, DGKZ, DNAJA2, DNAJB1, DPT, F9, FAM3B, FASLG, FCER2, FCGR2B, FCRL3, FES, FGF2, FGF5, FKBPlB, FKBP7, FLT3LG, FLT4, FMR1, FRZB, GDF2, GGA1, GNLY, GP2, GUSB, GZMA, HBEGF, HEBP1, HMBS, ICAM4, IFNG, IFNLR1, IGSF3, IKZF2, IL2RA, IL5RA, IL6, IL17D, IL18RAP, IL32, IL33, IL34, ING1, IRAK1, ITGA6, ITGB6, ITGB7, IVD, KEL, KIR2DL3, KLB, KLK1, KLK12, KLRD1, KRT19, LAIR2, LAP3, LBR, LEP, LPO, LSM1, MAPT, METAP1, MFAP3, MGMT, MITD1, MRPL46, MSMB, MYO9B, MZB1, NCAN, NINJ1, NMNAT1, NPM1, NPPB, NTF3, NUDC, ODAM, OGFR, OMD, PAEP, PDCD1, PDCD6, PDGFC, PFDN2, PKLR, PMVK, PQBP1, PRCP, PROK1, PRTFDC1, PSRC1, PSPN, PTPRM, RAB6B, RASA1, RCOR1, RRM2, SCGN, SERPINB9, SFTPA1, SIRT5, SIT1, SLAMF1, SLAMF6, SLITRK6, SMAD5, SMARCA2, SRP14, STIP1, STX16, SUGT1, SUSD1, TAFA5, TARBP2, TBC1D23, TBCB, TCL1B, TIA1, TNF, TNFRSF4, TNFRSF9, TNFRSF13C, TNFSF11, TNNI3, TPPP3, TSLP, TXLNA, UBAC1, UXS1, VIM, VSTM2L, VTCN1, VWF, WARS, WASF1, XRCC4, YTHDF3, ZBTB17, AGR2, CX3CL1, IDS, IL1B, RABGAPIL, SH2D1A, SMAD1 and/or WAS.

In certain embodiments, the panel of protein biomarkers comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 proteins selected from the biomarker group.

### 11—Protein Classifier

In certain embodiments, the panel of protein biomarkers comprises PAEP, SMARCA2, FAM3B, KEL, SLITRK6, ANXA10, TNFRSF9, ADCYAP1R1, DECR1, GDF2, and PDCD1.

In certain embodiments, the machine learning classifier model applies the following logistic regression function to the normalized protein expression values of the panel: Logit(P)=−1.420+0.980×PAEP+0.551×SMARCA2+0.348×FAM3B−0.883×KEL+0.607×SLITRK6−0.414×ANXA10+0.747×TNFRSF9−0.792×ADCYAP1R1+0.640×DECR1−0.433×GDF2+0.374×PDCD1.

In certain embodiments, the panel comprises PAEP and at least one additional protein selected from SMARCA2, FAM3B, KEL, SLITRK6, ANXA10, TNFRSF9, ADCYAP1R1, DECR1, GDF2, and PDCD1.

In certain embodiments, the panel comprises TNFRSF9 and at least one additional protein selected from PAEP, SMARCA2, FAM3B, KEL, SLITRK6, ANXA10, ADCYAP1R1, DECR1, GDF2, and PDCD1.

In certain embodiments, the panel comprises PDCD1 and at least one additional protein selected from PAEP, SMARCA2, FAM3B, KEL, SLITRK6, ANXA10, TNFRSF9, ADCYAP1R1, DECR1, and GDF2.

In certain embodiments, the panel comprises PAEP and TNFRSF9. In certain embodiments, the panel comprises PAEP, TNFRSF9, and PDCD1. In certain embodiments, the panel comprises PAEP, TNFRSF9, PDCD1, and SMARCA2. In certain embodiments, the panel comprises PAEP, TNFRSF9, PDCD1, SMARCA2, and DECR1. In certain embodiments, the panel comprises PAEP, TNFRSF9, PDCD1, SMARCA2, DECR1, and GDF2. In certain embodiments, the panel comprises PAEP, TNFRSF9, PDCD1, SMARCA2, DECR1, GDF2, and ANXA10. In certain embodiments, the panel comprises PAEP, TNFRSF9, PDCD1, SMARCA2, DECR1, GDF2, ANXA10, and FAM3B. In certain embodiments, the panel comprises PAEP, TNFRSF9, PDCD1, SMARCA2, DECR1, GDF2, ANXA10, FAM3B, and KEL. In certain embodiments, the panel comprises PAEP, TNFRSF9, PDCD1, SMARCA2, DECR1, GDF2, ANXA10, FAM3B, KEL, and SLITRK6. In certain embodiments, the panel comprises PAEP, TNFRSF9, PDCD1, SMARCA2, DECR1, GDF2, ANXA10, FAM3B, KEL, SLITRK6, and ADCYAP1R1.

In certain embodiments, the 14-protein candidate pool from which the 11-protein classifier is derived comprises PAEP, TBCB, PDCD1, SMARCA2, ADCYAP1R1, TNFRSF9, FAM3B, SLITRK6, ZBTB17, KEL, GDF2, ANXA10, SLAMF1, and DECR1. In certain embodiments, the panel comprises any 2, 3, 4, 5, 6, 7, 8, 9, 10, 11, 12, 13, or all 14 proteins from this candidate pool.

### 13—Protein Classifier

In certain embodiments, the panel of protein biomarkers comprises CX3CL1, IDS, IL1B, NCAN, ODAM, PAEP, PSPN, RABGAP1L, SH2D1A, SMAD1, SMAD5, TNFRSF9 and WAS.

In certain embodiments, the panel comprises CX3CL1 and at least one additional protein selected from IDS, IL1B, NCAN, ODAM, PAEP, PSPN, RABGAP1L, SH2D1A, SMAD1, SMAD5, TNFRSF9 and WAS.

In certain embodiments, the panel comprises IDS and at least one additional protein selected from CX3CL1, IL1B, NCAN, ODAM, PAEP, PSPN, RABGAP1L, SH2D1A, SMAD1, SMAD5, TNFRSF9 and WAS.

In certain embodiments, the panel comprises IL1B and at least one additional protein selected from CX3CL1, IDS, NCAN, ODAM, PAEP, PSPN, RABGAP1L, SH2D1A, SMAD1, SMAD5, TNFRSF9 and WAS.

In certain embodiments, the panel comprises CX3CL1 and IDS. In certain embodiments, the panel comprises CX3CL1, IDS, and IL1B. In certain embodiments, the panel comprises CX3CL1, IDS, IL1B, and NCAN. In certain embodiments, the panel comprises CX3CL1, IDS, IL1B, NCAN and ODAM. In certain embodiments, the panel comprises CX3CL1, IDS, IL1B, NCAN, ODAM and PAEP. In certain embodiments, the panel comprises CX3CL1, IDS, IL1B, NCAN, ODAM, PAEP and PSPN. In certain embodiments, the panel comprises CX3CL1, IDS, IL1B, NCAN, ODAM, PAEP, PSPN and RABGAP1L. In certain embodiments, the panel comprises CX3CL1, IDS, IL1B, NCAN, ODAM, PAEP, PSPN, RABGAP1L and SH2D1A. In certain embodiments, the panel comprises CX3CL1, IDS, IL1B, NCAN, ODAM, PAEP, PSPN, RABGAP1L, SH2D1A and SMAD1. In certain embodiments, the panel comprises CX3CL1, IDS, IL1B, NCAN, ODAM, PAEP, PSPN, RABGAP1L, SH2D1A, SMAD1 and SMAD5. In certain embodiments, the panel comprises CX3CL1, IDS, IL1B, NCAN, ODAM, PAEP, PSPN, RABGAP1L, SH2D1A, SMAD1, SMAD5 and TNFRSF9. In certain embodiments, the panel comprises any 1, 2, 3, 4, 5, 6, 7, 8, 9, 10, 11, 12 or all 13 proteins from this candidate pool.

### 15—Protein Classifier

In certain embodiments, the panel of protein biomarkers comprises PAEP, IL5RA, PSPN, CLEC6A, ANXA10, CD70, TAFA5, LPO, CEACAM3, CAPG, DCN, NCAN, TSLP, CFC1, and AGR2.

In certain embodiments, the panel comprises PAEP and at least one additional protein selected from IL5RA, PSPN, CLEC6A, ANXA10, CD70, TAFA5, LPO, CEACAM3, CAPG, DCN, NCAN, TSLP, CFC1, and AGR2.

In certain embodiments, the panel comprises CD70 and at least one additional protein selected from PAEP, IL5RA, PSPN, CLEC6A, ANXA10, TAFA5, LPO, CEACAM3, CAPG, DCN, NCAN, TSLP, CFC1, and AGR2.

In certain embodiments, the panel comprises ANXA10 and at least one additional protein selected from PAEP, IL5RA, PSPN, CLEC6A, CD70, TAFA5, LPO, CEACAM3, CAPG, DCN, NCAN, TSLP, CFC1, and AGR2.

In certain embodiments, the panel comprises PAEP and CD70. In certain embodiments, the panel comprises PAEP, CD70, and ANXA10. In certain embodiments, the panel comprises PAEP, CD70, ANXA10, and CLEC6A. In certain embodiments, the panel comprises PAEP, CD70, ANXA10, CLEC6A, and IL5RA. In certain embodiments, the panel comprises PAEP, CD70, ANXA10, CLEC6A, IL5RA, and TAFA5. In certain embodiments, the panel comprises PAEP, CD70, ANXA10, CLEC6A, IL5RA, TAFA5, and CEACAM3. In certain embodiments, the panel comprises PAEP, CD70, ANXA10, CLEC6A, IL5RA, TAFA5, CEACAM3, and CAPG. In certain embodiments, the panel comprises PAEP, CD70, ANXA10, CLEC6A, IL5RA, TAFA5, CEACAM3, CAPG, and DCN. In certain embodiments, the panel comprises PAEP, CD70, ANXA10, CLEC6A, IL5RA, TAFA5, CEACAM3, CAPG, DCN, and NCAN. In certain embodiments, the panel comprises PAEP, CD70, ANXA10, CLEC6A, IL5RA, TAFA5, CEACAM3, CAPG, DCN, NCAN, and LPO. In certain embodiments, the panel comprises PAEP, CD70, ANXA10, CLEC6A, IL5RA, TAFA5, CEACAM3, CAPG, DCN, NCAN, LPO, and PSPN. In certain embodiments, the panel comprises PAEP, CD70, ANXA10, CLEC6A, IL5RA, TAFA5, CEACAM3, CAPG, DCN, NCAN, LPO, PSPN, and TSLP. In certain embodiments, the panel comprises PAEP, CD70, ANXA10, CLEC6A, IL5RA, TAFA5, CEACAM3, CAPG, DCN, NCAN, LPO, PSPN, TSLP, and CFC1. In certain embodiments, the panel comprises PAEP, IL5RA, PSPN, CLEC6A, ANXA10, CD70, TAFA5, LPO, CEACAM3, CAPG, DCN, NCAN, TSLP, CFC1, and AGR2.

### 18—Protein Classifier

In certain embodiments, the panel of protein biomarkers comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.

In certain embodiments, the machine learning classifier model applies the following logistic regression function to the normalized protein expression values of the panel: logit(p)=−5.5935+1.5499 (TNFRSF9)+2.6571 (CD70)−3.7116 (TNFRSF13C)−5.3780 (PROK1)+1.5212 (CEACAM21)−1.1170 (PDCD1)+1.0400 (GZMA)+1.6626 (CCL2)−1.3980 (IL6)+0.2725 (FGF2)+0.8791 (CXCL10)−0.8435 (CXCL8)+0.9193 (IL17D)+1.1831 (KLRD1)−0.9768 (CD28)−2.1695 (CD79B)−0.3674 (FLT3LG)+2.9745 (HBEGF).

In certain embodiments, the panel comprises TNFRSF9 and at least one additional protein selected from CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.

In certain embodiments, the panel comprises GZMA and at least one additional protein selected from TNFRSF9, CD70, TNFRSF13C, PROK1, CEACAM21, PDCD1, CCL2, IL6, FGF2, CXCL10, CXCL8, IL17D, KLRD1, CD28, CD79B, FLT3LG, and HBEGF.

In certain embodiments, the panel comprises HBEGF and at least one additional protein selected from TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, and FLT3LG.

In certain embodiments, the panel comprises TNFRSF9 and GZMA. In certain embodiments, the panel comprises TNFRSF9, GZMA, and CXCL10. In certain embodiments, the panel comprises TNFRSF9, GZMA, CXCL10, and PDCD1. In certain embodiments, the panel comprises TNFRSF9, GZMA, CXCL10, PDCD1, and CCL2. In certain embodiments, the panel comprises TNFRSF9, GZMA, CXCL10, PDCD1, CCL2, and HBEGF. In certain embodiments, the panel comprises TNFRSF9, GZMA, CXCL10, PDCD1, CCL2, HBEGF, and FLT3LG. In certain embodiments, the panel comprises TNFRSF9, GZMA, CXCL10, PDCD1, CCL2, HBEGF, FLT3LG, and IL17D. In certain embodiments, the panel comprises TNFRSF9, GZMA, CXCL10, PDCD1, CCL2, HBEGF, FLT3LG, IL17D, and KLRD1. In certain embodiments, the panel comprises TNFRSF9, GZMA, CXCL10, PDCD1, CCL2, HBEGF, FLT3LG, IL17D, KLRD1, and CD70. In certain embodiments, the panel comprises TNFRSF9, GZMA, CXCL10, PDCD1, CCL2, HBEGF, FLT3LG, IL17D, KLRD1, CD70, and IL6. In certain embodiments, the panel comprises TNFRSF9, GZMA, CXCL10, PDCD1, CCL2, HBEGF, FLT3LG, IL17D, KLRD1, CD70, IL6, and CXCL8. In certain embodiments, the panel comprises TNFRSF9, GZMA, CXCL10, PDCD1, CCL2, HBEGF, FLT3LG, IL17D, KLRD1, CD70, IL6, CXCL8, and FGF2. In certain embodiments, the panel comprises TNFRSF9, GZMA, CXCL10, PDCD1, CCL2, HBEGF, FLT3LG, IL17D, KLRD1, CD70, IL6, CXCL8, FGF2, and CD28. In certain embodiments, the panel comprises TNFRSF9, GZMA, CXCL10, PDCD1, CCL2, HBEGF, FLT3LG, IL17D, KLRD1, CD70, IL6, CXCL8, FGF2, CD28, and CEACAM21. In certain embodiments, the panel comprises TNFRSF9, GZMA, CXCL10, PDCD1, CCL2, HBEGF, FLT3LG, IL17D, KLRD1, CD70, IL6, CXCL8, FGF2, CD28, CEACAM21, and TNFRSF13C. In certain embodiments, the panel comprises TNFRSF9, GZMA, CXCL10, PDCD1, CCL2, HBEGF, FLT3LG, IL17D, KLRD1, CD70, IL6, CXCL8, FGF2, CD28, CEACAM21, TNFRSF13C, and CD79B. In certain embodiments, the panel comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.

### Cross-Panel Combination Embodiments

In certain embodiments, the panel comprises proteins drawn from two or more of the representative panels described herein. In certain embodiments, the panel comprises CD70 and TNFRSF9, wherein CD70 is shared between the 15-protein and 18-protein representative panels. In certain embodiments, the panel comprises CD70, TNFRSF9, and PDCD1. In certain embodiments, the panel comprises CD70, TNFRSF9, PDCD1, and ANXA10. In certain embodiments, the panel comprises CD70, TNFRSF9, PDCD1, ANXA10, and GZMA. In certain embodiments, the panel comprises CD70, TNFRSF9, PDCD1, ANXA10, GZMA, and CCL2. In certain embodiments, the panel comprises CD70, TNFRSF9, PDCD1, ANXA10, GZMA, CCL2, and CXCL10. In certain embodiments, the panel comprises CD70, TNFRSF9, PDCD1, ANXA10, GZMA, CCL2, CXCL10, and PAEP. In certain embodiments, the panel comprises CD70, TNFRSF9, PDCD1, ANXA10, GZMA, CCL2, CXCL10, PAEP, and HBEGF. In certain embodiments, the panel comprises CD70, TNFRSF9, PDCD1, ANXA10, GZMA, CCL2, CXCL10, PAEP, HBEGF, and FLT3LG. In certain embodiments, the panel comprises CD70, TNFRSF9, PDCD1, ANXA10, GZMA, CCL2, CXCL10, PAEP, HBEGF, FLT3LG, and KLRD1.

In certain embodiments, the panel comprises proteins from the 11-protein classifier and the 18-protein classifier. In certain embodiments, such a panel comprises TNFRSF9, PDCD1, GZMA, and PAEP. In certain embodiments, such a panel further comprises one or more of DECR1, GDF2, ANXA10, CCL2, CXCL10, and HBEGF.

In certain embodiments, the panel comprises proteins from the 15-protein classifier and the 18-protein classifier. In certain embodiments, such a panel comprises CD70, GZMA, PDCD1, and CCL2. In certain embodiments, such a panel further comprises one or more of ANXA10, CXCL10, HBEGF, TNFRSF9, FLT3LG, and IL17D.

In certain embodiments, the panel comprises proteins from the 13-protein classifier, such panel comprises CX3CL1, IDS, IL1B, NCAN, ODAM, PAEP, PSPN, RABGAP1L, SH2D1A, SMAD1, SMAD5, TNFRSF9 and WAS. In certain embodiments, such a panel further comprises one or more, two or more, three or more, four or more, five or more, six or more, seven or more or eight or more of CX3CL1, IDS, IL1B, NCAN, ODAM, PAEP, PSPN, RABGAP1L, SH2D1A, SMAD1, SMAD5, TNFRSF9 and WAS.

### Immune Checkpoint and Tumor Microenvironment Subset Embodiments

In certain embodiments, the panel comprises one or more immune checkpoint-associated proteins selected from PDCD1, TNFRSF9, CD70, and CD28. In certain embodiments, the panel comprises PDCD1 and TNFRSF9. In certain embodiments, the panel comprises PDCD1, TNFRSF9, and CD70. In certain embodiments, the panel comprises PDCD1, TNFRSF9, CD70, and CD28. In certain embodiments, the panel further comprises one or more cytotoxicity-associated proteins selected from GZMA and KLRD1. In certain embodiments, the panel further comprises one or more chemokine-associated proteins selected from CCL2, CXCL8, CXCL10, and IL17D.

In certain embodiments, the panel comprises one or more angiogenesis-associated proteins selected from FGF2, HBEGF, and FLT3LG. In certain embodiments, the panel comprises FGF2 and HBEGF. In certain embodiments, the panel comprises FGF2, HBEGF, and FLT3LG. In certain embodiments, such a panel further comprises one or more of CXCL10, PDCD1, and TNFRSF9.

In certain embodiments, the panel comprises one or more tumor necrosis factor superfamily proteins selected from TNFRSF9, TNFRSF13C, TNFRSF4, and TNFSF11. In certain embodiments, the panel comprises TNFRSF9 and TNFRSF13C. In certain embodiments, the panel comprises TNFRSF9, TNFRSF13C, and CD70.

### Subset Embodiments

In certain embodiments, the panel comprises proteins having median estimated concentrations above 5 pg/mL in aqueous humor, as determined by regression-based calibration from qPCR-based proximity extension assay measurements. In certain embodiments, such proteins are selected from CCL2, CD28, GZMA, TNFRSF9, CXCL10, IL17D, FLT3LG, CXCL8, IL6, FGF2, and PDCD1. In certain embodiments, the panel comprises at least 3 proteins selected from CCL2, CD28, GZMA, TNFRSF9, CXCL10, IL17D, FLT3LG, CXCL8, IL6, FGF2, and PDCD1. In certain embodiments, the panel comprises at least 5 proteins selected from CCL2, CD28, GZMA, TNFRSF9, CXCL10, IL17D, FLT3LG, CXCL8, IL6, FGF2, and PDCD1. In certain embodiments, the panel comprises all 11 of CCL2, CD28, GZMA, TNFRSF9, CXCL10, IL17D, FLT3LG, CXCL8, IL6, FGF2, and PDCD1.

In certain embodiments, the panel comprises CCL2 and GZMA. In certain embodiments, the panel comprises CCL2, GZMA, and TNFRSF9. In certain embodiments, the panel comprises CCL2, GZMA, TNFRSF9, and CXCL10. In certain embodiments, the panel comprises CCL2, GZMA, TNFRSF9, CXCL10, and PDCD1. In certain embodiments, the panel comprises CCL2, GZMA, TNFRSF9, CXCL10, PDCD1, and IL17D. In certain embodiments, the panel comprises CCL2, GZMA, TNFRSF9, CXCL10, PDCD1, IL17D, and FLT3LG. In certain embodiments, the panel comprises CCL2, GZMA, TNFRSF9, CXCL10, PDCD1, IL17D, FLT3LG, and CXCL8. In certain embodiments, the panel comprises CCL2, GZMA, TNFRSF9, CXCL10, PDCD1, IL17D, FLT3LG, CXCL8, and IL6. In certain embodiments, the panel comprises CCL2, GZMA, TNFRSF9, CXCL10, PDCD1, IL17D, FLT3LG, CXCL8, IL6, and FGF2. In certain embodiments, the panel comprises CCL2, GZMA, TNFRSF9, CXCL10, PDCD1, IL17D, FLT3LG, CXCL8, IL6, FGF2, and CD28.

### Classifier Model Embodiments

In certain embodiments, the machine learning classifier model is a logistic regression model. In certain embodiments, the machine learning classifier model is a Random Forest classifier. In certain embodiments, the machine learning classifier model is a support vector machine classifier. In certain embodiments, the machine learning classifier model is trained using a feature selection process comprising LASSO regression with 10-fold cross-validation. In certain embodiments, the machine learning classifier model is trained using a feature selection process comprising Random Forest with 1,000 estimators. In certain embodiments, the machine learning classifier model is trained using a feature selection process comprising support vector machine-recursive feature elimination. In certain embodiments, the machine learning classifier model is trained using all three of LASSO regression, Random Forest, and support vector machine-recursive feature elimination, and the panel of protein biomarkers comprises proteins identified as informative by all three feature selection methods.

In certain embodiments, the machine learning classifier model is validated using five-fold stratified cross-validation. In certain embodiments, the machine learning classifier model achieves an area under the receiver operating characteristic curve of at least 0.7 in cross-validation. In certain embodiments, the machine learning classifier model achieves an area under the receiver operating characteristic curve of at least 0.8 in cross-validation. In certain embodiments, the machine learning classifier model achieves an area under the receiver operating characteristic curve of at least 0.9 in cross-validation. In certain embodiments, the machine learning classifier model achieves an area under the receiver operating characteristic curve of 1.0 in cross-validation.

In certain embodiments, classifying the subject comprises identifying the subject as having high metastatic risk when the metastatic risk score exceeds a predetermined probability threshold. In certain embodiments, the predetermined probability threshold is 0.50. In certain embodiments, the predetermined probability threshold is 0.55. In certain embodiments, the predetermined probability threshold is determined using Youden's index.

### Quantification Method Embodiments

In certain embodiments, quantifying protein expression levels comprises generating normalized protein expression values reported on a log 2 scale. In certain embodiments, quantifying protein expression levels further comprises generating extrapolated absolute concentration values in picograms per milliliter using regression-based calibration. In certain embodiments, the regression-based calibration uses paired qPCR-based proximity extension assay measurements as reference concentration values. In certain embodiments, the regression-based calibration achieves an R² value greater than 0.9 for calibration proteins. In certain embodiments, the multiplex proximity extension assay is performed using a dual-platform calibration comprising a next generation sequencing-based proximity extension assay and a qPCR-based proximity extension assay. In certain embodiments, the multiplex proximity extension assay quantifies protein expression levels for approximately 1,500 proteins present in the aqueous humor sample. In certain embodiments, the multiplex proximity extension assay is the Olink Explorer platform. In certain embodiments, the multiplex proximity extension assay is the Olink Flex platform.

### Application Embodiments

In certain embodiments, the method further comprises administering a treatment to the subject based on the classification. In certain embodiments, the treatment is selected from surgery, radiation therapy, plaque brachytherapy, photocoagulation, chemotherapy, immunotherapy, thermotherapy, and combinations thereof. In certain embodiments, the immunotherapy comprises administration of an immune checkpoint inhibitor. In certain embodiments, the immune checkpoint inhibitor targets PDCD1 (PD-1) or a ligand thereof. In certain embodiments, the immunotherapy comprises administration of a TNFRSF9 (4-1B) agonist.

In certain embodiments, the method further comprises directing the subject classified as having high metastatic risk to a hepatic surveillance protocol. In certain embodiments, the hepatic surveillance protocol comprises hepatic imaging at intervals of three months or less. In certain embodiments, the method further comprises directing the subject classified as having low metastatic risk to a standard surveillance protocol comprising hepatic imaging at intervals of six months or greater.

In certain embodiments, the method is applied to a subject who has not undergone tumor fine-needle aspiration biopsy. In certain embodiments, the method is applied to a subject for whom tumor fine-needle aspiration biopsy is contraindicated. In certain embodiments, the method is applied to a subject for whom tumor gene expression profile data is unavailable. In certain embodiments, the method provides a gene expression profile-equivalent risk classification in the absence of tumor-derived molecular data.

In certain embodiments, the method is performed at the time of diagnosis prior to any therapeutic intervention. In certain embodiments, the method is performed longitudinally at two or more time points during disease management to monitor changes in metastatic risk. In certain embodiments, the method is performed following local tumor treatment to assess residual molecular risk.

### Kit Embodiments

In certain embodiments, the invention provides a diagnostic kit for stratifying metastatic risk in a subject having uveal melanoma, the kit comprising a collection device configured to receive an aqueous humor sample, a multiplex proximity extension assay reagent system configured to quantify protein expression levels and generate normalized protein expression values, a panel of detection reagents for protein biomarkers as described in any of the panel embodiments herein, and a non-transitory computer-readable storage medium storing instructions which, when executed by a processor, cause the processor to receive the normalized protein expression values, apply a machine learning classifier model, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, to generate a metastatic risk score, and output the metastatic risk score. In certain embodiments, the kit further comprises reagents for regression-based calibration of absolute protein concentrations in picograms per milliliter. In certain embodiments, the kit further comprises a report generator configured to output a risk classification and associated probability score for clinical use.

### Statements

1. A method for stratifying metastatic risk in a subject having uveal melanoma, the method comprising:
   a) obtaining an aqueous humor sample from the subject;
   b) quantifying protein expression levels in the aqueous humor sample using an assay to generate normalized protein expression values for a plurality of proteins;
   c) applying a machine learning classifier model, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, to the normalized protein expression values of the panel to generate a metastatic risk score, wherein the panel comprises at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS;
   d) classifying the subject as having high metastatic risk or low metastatic risk based on the metastatic risk score; and
   e) directing the subject to a hepatic surveillance protocol when classified as having high metastatic risk or directing the subject to a standard surveillance protocol when classified as having low metastatic risk.
2. The method of statement 1, wherein the aqueous humor sample is collected from the subject prior to any therapeutic intervention for uveal melanoma.
3. The method of statement 1, wherein the aqueous humor sample is obtained by clear corneal paracentesis.
4. The method of statement 1, wherein b) further comprises generating extrapolated absolute concentration values in picograms per milliliter derived by regression-based calibration using reference concentration values.
5. The method of statement 4, wherein the regression-based calibration achieves an R² value greater than 0.9 for calibration proteins.
6. The method of statement 1, wherein a multiplex proximity extension assay is performed.
7. The method of statement 1, wherein the panel of protein biomarkers comprises PAEP, SMARCA2, FAM3B, KEL, SLITRK6, ANXA10, TNFRSF9, ADCYAP1R1, DECR1, GDF2, and PDCD1.
8. The method of statement 7, wherein the machine learning classifier model applies a logistic regression function expressed as:

Logit(P)=−1.420+0.980×PAEP+0.551×SMARCA2+0.348×FAM3B−0.883×KEL+0.607×SLITRK6−0.414×ANXA10+0.747×TNFRSF9−0.792×ADCYAP1R1+0.640×DECR1−0.433×GDF2+0.374×PDCD1.
9. The method of statement 1, wherein the panel of protein biomarkers comprises PAEP, IL5RA, PSPN, CLEC6A, ANXA10, CD70, TAFA5, LPO, CEACAM3, CAPG, DCN, NCAN, TSLP, CFC1, and AGR2 or wherein the biomarker panel comprises CX3CL1, IDS, IL1B, NCAN, ODAM, PAEP, PSPN, RABGAP1L, SH2D1A, SMAD1, SMAD5, TNFRSF9 and WAS.
10. The method of statement 1, wherein the panel of protein biomarkers comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.
11. The method of statement 10, wherein the machine learning classifier model applies a logistic regression function expressed as:

logit(p)=−5.5935+1.5499(TNFRSF9)+2.6571(CD70)−3.7116(TNFRSF13C)−5.3780(PROK1)+1.5212(CEACAM21)−1.1170(PDCD1)+1.0400(GZMA)+1.6626(CCL2)−1.3980(IL6)+0.2725(FGF2)+0.8791(CXCL10)−0.8435(CXCL8)+0.9193(IL17D)+1.1831(KLRD1)−0.9768(CD28)−2.1695(CD79B)−0.3674(FLT3LG)+2.9745(HBEGF).
12. The method of statement 1, wherein the machine learning classifier model is a logistic regression model.
13. The method of statement 1, wherein the machine learning classifier model is trained using a feature selection process selected from the group consisting of least absolute shrinkage and selection operator (LASSO) regression, Random Forest, support vector machine-recursive feature elimination (SVM-RFE), and combinations thereof.
14. The method of statement 1, wherein the machine learning classifier model achieves an area under the receiver operating characteristic curve of at least 0.9 in five-fold stratified cross-validation.
15. The method of statement 1, wherein classifying the subject comprises identifying the subject as having high metastatic risk when the metastatic risk score exceeds a predetermined probability threshold and identifying the subject as having low metastatic risk when the metastatic risk score is below the predetermined probability threshold.
16. The method of statement 15, wherein the predetermined probability threshold is 0.50.
17. The method of statement 15, wherein the predetermined probability threshold is 0.55.
18. The method of statement 15, wherein the predetermined probability threshold is determined using Youden's index.
19. The method of statement 1, wherein the subject has not undergone fine-needle aspiration biopsy of the uveal melanoma tumor, and wherein the metastatic risk score provides a gene expression profile-equivalent classification in the absence of tumor-derived molecular data.
20. A method for determining metastatic risk in a subject having uveal melanoma for whom tumor-derived gene expression profile data is unavailable, the method comprising:
   a) obtaining an aqueous humor sample from the subject;
   b) quantifying protein expression levels in the aqueous humor sample using an assay to generate normalized protein expression values for a panel of protein biomarkers comprising TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF;
   c) applying a logistic regression classifier trained on aqueous humor samples from subjects with known gene expression profile classifications to the normalized protein expression values to generate a predicted probability of high metastatic risk;
   d) classifying the subject as having a protein expression profile of PEP1 (low metastatic risk) when the predicted probability is below 0.50 and as having a protein expression profile of PEP2 (high metastatic risk) when the predicted probability is at or above 0.50; and
   e) directing the subject to a surveillance or treatment protocol based on the classification.
21. The method of statement 20, wherein the subject has not undergone fine-needle aspiration biopsy of the uveal melanoma tumor.
22. The method of statement 20, wherein the logistic regression classifier applies the function:

logit(p)=−5.5935+1.5499(TNFRSF9)+2.6571(CD70)−3.7116(TNFRSF13C)−5.3780(PROK1)+1.5212(CEACAM21)−1.1170(PDCD1)+1.0400(GZMA)+1.6626(CCL2)−1.3980(IL6)+0.2725(FGF2)+0.8791(CXCL10)−0.8435(CXCL8)+0.9193(IL17D)+1.1831(KLRD1)−0.9768(CD28)−2.1695(CD79B)−0.3674(FLT3LG)+2.9745(HBEGF).
23. A method for treating a subject having uveal melanoma, the method comprising:
   a) obtaining an aqueous humor sample from the subject;
   b) quantifying protein expression levels in the aqueous humor sample using an assay to generate normalized protein expression values for a panel of protein biomarkers comprising at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1 and WAS;
   c) applying a machine learning classifier model (e.g., wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma) to the normalized protein expression values to generate a metastatic risk score;
   d) classifying the subject as having high metastatic risk or low metastatic risk based on the metastatic risk score; and
   e) administering a treatment to the subject based on the classification, wherein the treatment is selected from the group consisting of surgery, radiation therapy, plaque brachytherapy, photocoagulation, chemotherapy, immunotherapy, thermotherapy, and combinations thereof.
24. The method of statement 23, wherein the treatment comprises immunotherapy, and wherein the immunotherapy comprises administration of an immune checkpoint inhibitor.
25. The method of statement 24, wherein the immune checkpoint inhibitor targets an immune checkpoint protein selected from PDCD1, TNFRSF9, and CD70.
26. A method of monitoring metastatic risk in a subject having uveal melanoma over time, the method comprising:
   a) obtaining a first aqueous humor sample from the subject at a first time point and a second aqueous humor sample from the subject at a second time point;
   b) quantifying protein expression levels in each aqueous humor sample to generate normalized protein expression values for a panel of protein biomarkers comprising at least five protein biomarkers selected from the group recited in claim 1;
   c) applying a machine learning classifier model, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma, to the normalized protein expression values to generate a first metastatic risk score for the first aqueous humor sample and a second metastatic risk score for the second aqueous humor sample; and
   d) comparing the first metastatic risk score and the second metastatic risk score to assess changes in metastatic risk over time.
27. A method for generating a metastatic risk classifier for uveal melanoma, the method comprising:
   a) obtaining aqueous humor samples from a training cohort of subjects having uveal melanoma, wherein the training cohort includes subjects with known gene expression profile classifications comprising GEP1 (low metastatic risk) and GEP2 (high metastatic risk);
   b) quantifying protein expression levels in the aqueous humor samples;
   c) selecting a panel of protein biomarkers from the quantified protein expression levels by applying at least one feature selection method selected from the group consisting of LASSO regression, Random Forest, and support vector machine-recursive feature elimination, wherein the selected protein biomarkers are differentially expressed between GEP1 and GEP2 uveal melanoma tumors; and
   d) training a machine learning classifier model on the protein expression values of the selected panel to generate a metastatic risk classifier, optionally, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma.
28. The method of statement 27, wherein c) comprises applying each of LASSO regression, Random Forest, and support vector machine-recursive feature elimination, and selecting protein biomarkers identified as informative by all three feature selection methods.
29. The method of statement 27, wherein the machine learning classifier model is validated using five-fold stratified cross-validation.
30. The method of statement 27, further comprising deploying the trained metastatic risk classifier to classify an aqueous humor sample from a subject not included in the training cohort, and outputting a metastatic risk classification directing the subject to a surveillance or treatment protocol.
31. A computer-implemented method for generating a metastatic risk score for a subject having uveal melanoma, the method comprising:
   a) receiving, by a processor, normalized protein expression values for a panel of protein biomarkers quantified from an aqueous humor sample from the subject, wherein the panel comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF;
   b) applying, by the processor, a logistic regression classifier to the normalized protein expression values to generate a predicted probability of high metastatic risk; and
   c) outputting, by the processor, a classification of high metastatic risk when the predicted probability is at or above a predetermined threshold and a classification of low metastatic risk when the predicted probability is below the predetermined threshold, wherein the classification directs a clinical action selected from hepatic surveillance, treatment selection, and clinical trial enrollment.
32. A set of detection reagents for stratifying metastatic risk in uveal melanoma, comprising pairs of oligonucleotide-conjugated antibodies specific for each of TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF, wherein each pair of oligonucleotide-conjugated antibodies is configured to bind its respective target protein in proximity such that the conjugated oligonucleotides hybridize and serve as templates for extension by a DNA polymerase.
33. A diagnostic kit for stratifying metastatic risk in a subject having uveal melanoma, the kit comprising:
   a) a collection device configured to receive an aqueous humor sample from the subject;
   b) a multiplex proximity extension assay reagent system configured to quantify protein expression levels in the aqueous humor sample;
   c) a panel of detection reagents for protein biomarkers comprising at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, and HBEGF; and
   d) a non-transitory computer-readable storage medium storing instructions which, when executed by a processor, cause the processor to:
      i) receive protein expression values generated by the multiplex proximity extension assay reagent system;
      ii) apply a machine learning classifier model trained on aqueous humor samples from subjects with known uveal melanoma metastatic risk classifications to the protein expression values to generate a metastatic risk score and/or and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma; and
      iii) output a clinical recommendation based on the metastatic risk score, wherein the clinical recommendation comprises a hepatic surveillance interval, a candidate treatment modality, or a clinical trial eligibility determination.
33a. A diagnostic kit for stratifying metastatic risk in a subject having uveal melanoma, the kit comprising:
   a) a collection device configured to receive an aqueous humor sample from the subject;
   b) an assay reagent system configured to quantify protein expression levels in the aqueous humor sample;
   c) a panel of detection reagents for protein biomarkers comprising at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, and HBEGF; and
   d) a non-transitory computer-readable storage medium storing instructions which, when executed by a processor, cause the processor to:
      i) receive protein expression values generated by the multiplex proximity extension assay reagent system;
      ii) apply a machine learning classifier to the protein expression values to generate a metastatic risk score, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma; and
      iii) output a clinical recommendation based on the metastatic risk score, wherein the clinical recommendation comprises a hepatic surveillance interval, a candidate treatment modality, or a clinical trial eligibility determination.
34. The diagnostic kit of statement 33, wherein the panel of detection reagents comprises detection reagents for the protein biomarkers PAEP, SMARCA2, FAM3B, KEL, SLITRK6, ANXA10, TNFRSF9, ADCYAP1R1, DECR1, GDF2, and PDCD1.
35. The diagnostic kit of statement 33, wherein the panel of detection reagents comprises detection reagents for the protein biomarkers PAEP, IL5RA, PSPN, CLEC6A, ANXA10, CD70, TAFA5, LPO, CEACAM3, CAPG, DCN, NCAN, TSLP, CFC1, and AGR2.
36. The diagnostic kit of statement 33, wherein the panel of detection reagents comprises detection reagents for the protein biomarkers TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.
37. A composition comprising an aqueous humor sample obtained from a subject having uveal melanoma, wherein the aqueous humor sample comprises at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS each bound to a pair of oligonucleotide-conjugated antibodies specific for the respective protein biomarker, wherein the oligonucleotide-conjugated antibodies in each pair hybridize to one another when bound to the respective protein biomarker in proximity and serve as templates for extension by a DNA polymerase.
37a. A composition comprising an aqueous humor sample obtained from a subject having uveal melanoma, wherein the aqueous humor sample comprises at least five protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS.
38. The composition of statement 37, wherein the aqueous humor sample has been obtained from the subject by clear corneal paracentesis and comprises a volume of from about 0.05 mL to about 0.2 mL.
39. The composition of statement 37, wherein the at least five protein biomarkers comprise TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF, each bound to a pair of oligonucleotide-conjugated antibodies specific for the respective protein biomarker.
40. The composition of statement 37, wherein the at least five protein biomarkers comprise CX3CL1, IDS, IL1B, NCAN, ODAM, PAEP, PSPN, RABGAP1L, SH2D1A, SMAD1, SMAD5, TNFRSF9 and WAS, each bound to a pair of oligonucleotide-conjugated antibodies specific for the respective protein biomarker.
41. The composition of statement 37, wherein the at least five protein biomarkers comprise PAEP, SMARCA2, FAM3B, KEL, SLITRK6, ANXA10, TNFRSF9, ADCYAP1R1, DECR1, GDF2, and PDCD1, each bound to a pair of oligonucleotide-conjugated antibodies specific for the respective protein biomarker.
42. The composition of statement 37, further comprising a DNA polymerase, deoxyribonucleoside triphosphates, and a buffer suitable for oligonucleotide extension.
43. The composition of statement 37, wherein the aqueous humor sample further comprises an extended oligonucleotide product generated by hybridization and extension of the oligonucleotide-conjugated antibody pairs bound to the at least five protein biomarkers.
44. A kit for stratifying metastatic risk in a subject having uveal melanoma, the kit comprising:
   a) a collection device configured to obtain an aqueous humor sample from the subject by clear corneal paracentesis;
   b) a sample container configured to receive and store the aqueous humor sample under conditions maintaining protein stability;
   c) a panel of detection reagents configured to quantify protein expression levels of at least five protein biomarkers in the aqueous humor sample, wherein the at least five protein biomarkers are selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS, wherein the panel of detection reagents comprises pairs of oligonucleotide-conjugated antibodies specific for each of the at least five protein biomarkers;
   d) reagents for performing a multiplex proximity extension assay, comprising a DNA polymerase, deoxyribonucleoside triphosphates, and a buffer suitable for oligonucleotide extension;
   e) optionally, reagents for amplifying and detecting extended oligonucleotide products by next generation sequencing, quantitative polymerase chain reaction, or both; and
   f) instructions for obtaining the aqueous humor sample, performing the multiplex proximity extension assay, and stratifying the subject as having high metastatic risk or low metastatic risk based on the protein expression levels of the at least five protein biomarkers.
44a. A kit for stratifying metastatic risk in a subject having uveal melanoma, the kit comprising:
   a) a collection device configured to obtain an aqueous humor sample from the subject by clear corneal paracentesis;
   b) a sample container configured to receive and store the aqueous humor sample under conditions maintaining protein stability;
   c) a panel of detection reagents configured to quantify protein expression levels of at least two, at least three, at least four, at least five protein biomarkers in the aqueous humor sample, wherein the at least five protein biomarkers are selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, RABGAP1L, SH2D1A, SMAD1, and WAS; and
   d) instructions for obtaining the aqueous humor sample, performing the multiplex proximity extension assay, and stratifying the subject as having high metastatic risk or low metastatic risk based on the protein expression levels of the at least five protein biomarkers.
45. The kit of statement 44, wherein the panel of detection reagents comprises pairs of oligonucleotide-conjugated antibodies specific for the protein biomarkers TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.
46. The kit of statement 44, wherein the panel of detection reagents comprises pairs of oligonucleotide-conjugated antibodies specific for the protein biomarkers PAEP, SMARCA2, FAM3B, KEL, SLITRK6, ANXA10, TNFRSF9, ADCYAP1R1, DECR1, GDF2, and PDCD1.
47. The kit of statement 44, wherein the panel of detection reagents comprises pairs of oligonucleotide-conjugated antibodies specific for the protein biomarkers PAEP, SMARCA2, CX3CL1, IDS, IL1B, NCAN, ODAM, PAEP, PSPN, RABGAP1L, SH2D1A, SMAD1, SMAD5, TNFRSF9 and WAS.
48. The kit of statement 44, wherein the panel of detection reagents comprises pairs of oligonucleotide-conjugated antibodies specific for the protein biomarkers PAEP, IL5RA, PSPN, CLEC6A, ANXA10, CD70, TAFA5, LPO, CEACAM3, CAPG, DCN, NCAN, TSLP, CFC1, and AGR2.
49. The kit of statement 44, further comprising a non-transitory computer-readable storage medium storing instructions which, when executed by a processor, cause the processor to:
   i) receive protein expression values generated by the multiplex proximity extension assay;
   ii) apply a logistic regression classifier trained on aqueous humor samples from subjects with known gene expression profile classifications to the protein expression values to generate a metastatic risk score; and
   iii) output a classification of the subject as having high metastatic risk when the metastatic risk score is at or above a predetermined threshold, or as having low metastatic risk when the metastatic risk score is below the predetermined threshold.
50. The kit of claim 49, wherein the logistic regression classifier applies the function:

logit(p)=−5.5935+1.5499(TNFRSF9)+2.6571(CD70)−3.7116(TNFRSF13C)−5.3780(PROK1)+1.5212(CEACAM21)−1.1170(PDCD1)+1.0400(GZMA)+1.6626(CCL2)−1.3980(IL6)+0.2725(FGF2)+0.8791(CXCL10)−0.8435(CXCL8)+0.9193(IL17D)+1.1831(KLRD1)−0.9768(CD28)−2.1695(CD79B)−0.3674(FLT3LG)+2.9745(HBEGF).

Without further elaboration, it is believed that one skilled in the art, using the preceding description, can utilize the present invention to the fullest extent. The following examples are illustrative only and not limiting of the remainder of the disclosure in any way whatsoever.

The following examples are put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I

### Methods

### Biological Source

Aqueous humor collected at diagnosis from UM patients, prior to treatment initiation.

### Proteomic Platform

Proximity Extension Assay (PEA) used for high-sensitivity, multiplex protein quantification.

Includes both normalized expression (NPX) and extrapolated absolute concentration (pg/mL) analyses.

Dual-platform calibration (NGS and qPCR-based PEA) allows batch normalization and absolute value extrapolation.

### Biomarker Panel

The following proteins were identified as differentially expressed between GEP1 and GEP2 UM tumors using PEA technology (each entry includes the UniProt ID for precise reference): ACP5—P13686; ADAM22—Q9POK1; ADCYAP1R1—P41586; ADH4—P08319; AGXT—P21549; AHSP—Q9NZD4; AIF1—P55008; AMBP—P02760; ANXA10—Q9UJ72; ANXA4—P09525; APLP1—P51693; ARG1—P05089; ATF2—P15336; AXIN1—015169; BANK1—Q8ND1B2; BIRC2—Q13489; BRK1—Q9HOK1; CA1—P00915; CALCOCO1—Q9UPU3; CASP1—P29466; CASP10—Q92851; CASP2—P42575; CCL24—000175; CCL25—015444; CCL8—P80075; CD27—P26842; CD33—P20138; CD6—P30203; CD70—P32970; CD74—P04233; CD79B—P40259; CDNF—Q9H2CO; CEACAM3—Q8WWL7; CEBPB—P17676; CEP164—Q9UPVO; CES3—000748; CLEC4A—Q9NZC2; CLIP2—Q9Y2J4; CLSPN—Q9HAW4; CNTN3—Q9P232; CRHR1—P34998; CRX—043186; CSF3—P09919; CXCL13—043927; DAPP1—Q9NP31; DCTN1—Q14203; DECR1—Q16698; DGKZ—Q13574; DNAJA2—P25685; DNAJB1—P25685; DPT—P51858; F9—P00740; FAM3B—Q96BQ1; FASLG—P48023; FCER2—P06734; FCGR2B—P31994; FCRL3—Q96LA6; FES—P07332; FGF5—P12034; FKBP1B—Q13451; FKBP7—Q9Y680; FLT4—P35916; FMR1—Q06787; FRZB—Q92765; GDF2—Q9UK05; GGA1—Q9UJY5; GNLY—P22749; GP2—P55259; GUSB—P08236; GZMA—P12544; HEBP1—Q9Y2J8; HMBS—P08397; ICAM4—Q14773; IFNG—P01579; IFNLR1—Q8IU57; IGSF3—Q9H6X2; IKZF2—Q9UKT9; IL18RAP—Q9NPH3; IL2RA—P01589; IL32—Q9H293; IL33—095760; IL34—Q6ZMJ4; IL5—P05113; ING1—Q9UK53; IRAK1—P51617; ITGA6—P23229; ITGB6—P18564; ITGB7—P26010; IVD—P26440; KEL—P23276; KIR2DL3—P43631; KLB—Q86Z14; KLK1—P06870; KLK12—Q9UKR3; KRT19—P08727; LAIR2—Q6GTX8; LAP3—P30038; LBR—Q14739; LEP—P41159; LSM1—015116; MAPT—P10636; METAP1—P53582; MFAP3—Q9UM44; MGMT—P16455; MITD1—Q9HOA8; MRPL46—Q9Y3D6; MSMB—P08118; MYO9B—Q13459; MZB1—Q96BJ3; NINJ1—076076; NMNAT1—Q9HAN9; NPM1—P06748; NPPB—P16860; NTF3—P20783; NUDC—Q9Y266; ODAM—Q8N6F7; OGFR—Q9NZT2; OMD—Q99983; PAEP—P09466; PDCD1—Q15116; PDCD6—075340; PDGFC—Q9NRA1; PFDN2—Q9UHV9; PKLR—P30613; PMVK—Q15126; PQBP1—060828; PRCP—P42785; PROK1—Q9HC23; PRTFDC1—Q9HOU3; PSRC1—Q96B26; PTPRM—Q92835; RAB6B—Q9NRW1; RASA1—P20936; RCOR1—Q9UKLO; RRM2—P31350; SCGN—076038; SERPINB9—P50453; SFTPA1—P35247; SIRT5—Q9NXA8; SIT1—Q9P1W8; SLAMF1—Q13291; SLAMF6—Q9HBE5; SLITRK6—Q9H6Y7; SMAD5—Q99717; SMARCA2—P51531; SRP14—P37108; STIP1—P31948; STX16—Q9H3P7; SUGT1—Q9Y2Z0; SUSD1—Q8NOW4; TACC3—Q9Y6A5; TAFA5—Q8N8I0; TARBP2—Q15633; TBC1D23—Q8N9I0; TBCB—Q99426; TCL1B—Q9HOU6; TIA1—P31483; TNF.2—P01375; TNF.3—P01375; TNFRSF4—P43489; TNFRSF9—Q07011; TNFSF11—014788; TNNI3—P19429; TPPP3—Q9BUB4; TXLNA—Q9Y6KO; UBAC1—Q9Y2L9; UXS1—Q9Y2C3; VIM—P08670; VSTM2L—Q6UXB8; VTCN1—Q7Z7D3; VWF—P04275; WARS—P23381; WASF1—Q92558; XRCC4—Q13426; YTHDF3—Q7Z739; ZBTB17—Q13105; CX3CL1—P78423; IDS—P22304; IL1B—P01584; RABGAP1L—Q5R372; SH2D1A—060880; SMAD1—Q15797; and WAS—P42768.

Reference to each protein by gene symbol or UniProt identifier is intended to encompass the full-length mature human protein sequence as deposited in the UniProt database as of the priority date, including any isoforms or post-translationally modified forms thereof. All DNA and protein sequences are hereby incorporated by reference.

### Classifier Model

Logistic regression model trained using selected proteins achieved an AUC of 1.0 in cross-validation.

ROC curve analysis for individual DEPs confirmed diagnostic potential with several proteins exceeding AUC thresholds of 0.7.

A clinical concentration threshold of ≥5 pg/mL was used to assess feasibility for assay development.

### Supporting Data

Aqueous humor samples from >70 UM patients analyzed.

Dual-platform (NGS and qPCR) PEA applied to enable both normalized and absolute quantification.

Regression models for AQ extrapolation yielded R²>0.9 for calibration proteins.

Classifier validated via five-fold cross-validation and individual ROC curve analyses.

### Example II

Machine Learning-Driven Protein Signature Predicts Metastatic Risk in Uveal Melanoma from Aqueous Humor

### Introduction

Uveal melanoma (UM) is a rare but highly aggressive intraocular malignancy, with nearly half of all patients developing systemic metastases-primarily to the liver-despite effective local control of the primary tumor. Accurate stratification of metastatic risk at diagnosis is essential for tailoring surveillance strategies, guiding therapeutic decisions, and improving long-term outcomes. Gene expression profiling (GEP), which classifies tumors into low-risk (GEP1) and high-risk (GEP2) groups based on transcriptional signatures, remains the current clinical standard. However, GEP requires intraocular tumor biopsy, an invasive procedure associated with potential complications including retinal detachment, hemorrhage, and tumor seeding, and is often limited by tumor size or accessibility.

Liquid biopsy approaches offer an appealing alternative for molecular profiling in UM, and the aqueous humor (AH)—a clear fluid in the anterior chamber of the eye-has emerged as a promising diagnostic medium. A previous study (PMC10094875) demonstrated that multiplex proteomic analysis of AH could uncover molecular subtypes of UM, revealing distinct immunological and stromal signaling signatures that reflected tumor heterogeneity. That work established the feasibility of using AH proteomics to infer intraocular tumor biology non-invasively, advancing the concept of the eye as a privileged site for liquid biopsy in ophthalmic oncology.

However, while the earlier study successfully characterized molecular subtypes and highlighted the presence of biologically meaningful proteomic variation in AH, it was not designed to produce patient-level predictive models. Specifically, it did not evaluate whether AH protein profiles could be used to infer GEP-based metastatic risk with clinical accuracy, nor did it address the practical challenge of applying such models to real-world diagnostic cases lacking GEP data. Bridging this gap is critical for translating AH proteomics into a reliable clinical tool for risk stratification-especially in scenarios where biopsy is not feasible or GEP results are inconclusive or unavailable.

In this follow-up study, high-throughput proteomic profiling was leveraged and multiple machine learning approaches were applied to identify a minimal protein signature in AH that can distinguish between GEP1 and GEP2 tumors with high accuracy. By integrating LASSO regression, Random Forest, and support vector machine-based feature selection, a robust classifier was derived and its performance was validated using five-fold cross-validation. The model's clinical applicability is demonstrated by applying it to a set of GEP-unknown AH samples, simulating a real-world application of this methodology for risk prediction in the absence of biopsy-derived GEP data.

This study builds directly upon previous work by transitioning from discovery-based proteomic subtyping to predictive modeling for clinical risk classification. In doing so, a minimally invasive, scalable, and actionable tool was established for metastatic risk assessment in UM—providing clinicians with a powerful adjunct or alternative to conventional biopsy-based methods and paving the way for personalized, risk-informed patient management.

### Material and Methods

### Patient Cohort and Sample Collection

A total of 69 aqueous humor (AH) samples were collected at the time of diagnosis from patients with uveal melanoma prior to any therapeutic intervention. All samples were obtained under institutional review board (IRB)-approved protocols at the Keck School of Medicine, University of Southern California. Gene expression profile (GEP) classification was available for 47 samples, including 32 classified as GEP1 (low risk) and 15 as GEP2 (high risk). The remaining 22 samples lacked GEP data and were categorized as GEP-unknown. All AH samples were de-identified and stored at −80 °C until proteomic analysis.

### Proteomic Profiling

Protein expression in AH samples was quantified using the Olink® Explorer platform, which employs proximity extension assay (PEA) technology for multiplexed, high-sensitivity protein detection. Each sample was profiled for approximately 1,500 proteins spanning several biological pathways. Protein abundance was reported as Normalized Protein eXpression (NPX) values, log 2-transformed as per the standard Olink analytical pipeline.

### Feature Selection

To identify proteins most predictive of GEP classification, three independent machine learning-based feature selection methods were applied: LASSO regression with 10-fold cross-validation to penalize non-informative features, Random Forest to assess feature importance based on mean decrease in impurity using 1,000 estimators, and Support Vector Machine with Recursive Feature Elimination (SVM-RFE) to rank proteins by their contribution to the classification boundary. The top 100 features from each method were compared, and 14 overlapping proteins consistently identified across all three approaches were selected for downstream model development.

### Model Development and Validation

A logistic regression model was constructed using 11 proteins refined from the initial 14-protein set via forward feature selection. The model was trained on the 47 GEP-classified samples using five-fold stratified cross-validation. Predictive performance was evaluated by calculating the area under the receiver operating characteristic curve (AUC), and an optimal probability threshold for classification was determined using Youden's index.

### Application to GEP-Unknown Samples

The trained model was applied to the 22 GEP-unknown AH samples. Based on the probability output from the logistic regression model, each sample was classified as either GEP1 or GEP2 using the optimized threshold. This step enabled inference of metastatic risk without the need for tumor-derived GEP data.

### Data Analysis and Visualization

All analyses were conducted using Python version 3.9 with the scikit-learn and statsmodels libraries. Data visualization, including principal component analysis (PCA), receiver operating characteristic (ROC) curves, and feature importance plots, was performed using matplotlib and seaborn.

### Results

Comparative analysis between 32 GEP1 and 15 GEP2 patients revealed statistically significant differences in preferentially expressed antigen in melanoma (PRAME) status (p=0.038), American Joint Committee on Cancer (AJCC) stage (p<0.001), clinical tumor stage (p<0.001), and BRCA1-associated protein 1 (BAP1) mutation status (p<0.001).

Among GEP1 patients, 16.1% were PRAME-positive, compared to 46.6% (7/15) of GEP2 patients. AJCC staging indicated that 93.5% (29/31) of GEP1 tumors were stage I or II, while 53% (8/15) of GEP2 tumors presented at more advanced stages. Similarly, GEP2 tumors exhibited later clinical staging more frequently than GEP1 tumors. Notably, BAP1 mutations were identified in 60% (9/15) of GEP2 tumors, versus only 6.4% (2/31) in GEP1 tumors.

To assess the potential of AH proteins as biomarkers of metastatic risk in UM, proteomic profiling was performed on all diagnostic AH samples. Quantification of approximately 1,500 proteins was conducted using multiplex Proximity Extension Assay (PEA) platform (Olink). Protein expression was normalized as NPX (log 2 scale) using the Olink analytical pipeline. Differential protein expressions were assessed between GEP classes. To identify informative proteins, machine learning methods were applied—LASSO regression, Random Forest, and Support Vector Machine Recursive Feature Elimination (SVM-RFE)—to rank proteins based on their contribution to GEP class differentiation.

### Cross-Comparison of Feature Rankings Revealed a Shared Set of 14 Informative Proteins

PAEP, TBCB, PDCD1, SMARCA2, ADCYAP1R1, TNFRSF9, FAM3B, SLITRK6, ZBTB17, KEL, GDF2, ANXA10, SLAMF1, and DECR1. LASSO coefficients and Random Forest feature importance scores confirmed the relevance of these proteins. Among them, 9 of the 14 proteins were significantly upregulated in GEP2 samples. Principal component analysis (PCA) of the 14-protein expression profiles demonstrated distinct clustering between GEP1 and GEP2 samples.

To evaluate their predictive performance, a logistic regression model incorporating the identified proteins was constructed. Through forward feature selection, a refined panel of 11 proteins was found to achieve a mean AUC of 1.0 in five-fold cross-validation. The final model was expressed as:

Logit(P)=−1.420+0.980×PAEP+0.551×SMARCA2+0.348 XFAM3B−0.883 XKEL+0.607×SLITRK6−0.414×ANXA10+0.747×TNFRSF9−0.792×ADCYAP1R1+0.640×DECR1−0.433×GDF2+0.374×PDCD1

At an optimized threshold of 0.55, determined by Youden's index, this model correctly classified all GEP2 samples. Application of the model to 22 GEP-unknown AH samples enabled inference of metastatic potential based on AH protein expression profiles.

### Methods

### Patient Samples and Classification

A total of 69 aqueous humor (AH) samples were collected at diagnosis from patients with uveal melanoma (UM) prior to initiation of therapy. Gene expression profile (GEP) classification, performed by Castle Biosciences, was available for 47 samples: 32 GEP1 (low metastatic risk) and 15 GEP2 (high metastatic risk). The remaining 22 samples lacked GEP data and were categorized as GEP-unknown (GEP NA). All specimens were obtained under institutional review board (RB)-approved protocols, de-identified, and processed at the Keck School of Medicine, University of Southern California (USC).

### Proteomic Analysis

Protein profiling was performed using the Olink® Explorer platform, which utilizes proximity extension assay (PEA) technology for high-throughput, multiplexed quantification of relative protein levels. Protein expression values were normalized and reported as Normalized Protein eXpression (NPX) on a log 2 scale, as generated by the Olink analysis pipeline.

### Feature Selection and Model Development

To identify proteins predictive of GEP classification, three machine learning-based feature selection algorithms were employed: LASSO regression, with 10-fold cross-validation, to penalize non-informative features. Random Forest, ranking protein importance based on mean decrease in impurity across 1,000 estimators. Support Vector Machine-Recursive Feature Elimination (SVM-RFE), ranking proteins based on their contribution to the SVM decision boundary.

The top 100 proteins from each method were compared, and 14 overlapping proteins identified across all three approaches were selected for model development.

### Logistic Regression and Validation

A logistic regression model was constructed using a minimal set of 11 proteins, selected from an initial pool of 14 informative candidates through forward feature selection. This iterative approach identified the subset of proteins that optimized model performance while minimizing complexity. Model training and performance evaluation were conducted using Python (v3.9) with the scikit-learn library. Five-fold cross-validation with stratified sampling was applied to assess predictive accuracy, yielding a mean area under the curve (AUC) of 1.0. An optimal probability threshold of 0.55 was determined using Youden's index.

### Application to GEP-Unknown Samples

The finalized model was applied to the 22 GEP-unknown AH samples to predict metastatic potential. Probabilities for GEP2 classification were computed, and samples with predicted probabilities exceeding 0.55 were classified as high-risk.

### Discussion

This study demonstrates that an aqueous humor (AH)-based proteomic signature can serve as a reliable, minimally invasive tool for metastatic risk stratification in uveal melanoma (UM). Through high-throughput proteomic profiling and integration of multiple machine learning algorithms, we developed and validated an 11-protein classifier that discriminates GEP1 (low-risk) from GEP2 (high-risk) tumors with high fidelity (AUC=1.0). The model's application to GEP-unknown samples illustrates its translational utility for patients in whom tumor biopsy is not feasible or GEP data are unavailable.

The findings reinforce the role of the tumor microenvironment (TME) in UM progression. Several proteins in the classifier—such as PDCD1 (PD-1), TNFRSF9 (4-1BB), and GDF2 (BMP9)—are key immune modulators, supporting the concept that immune regulation is a driving force in metastatic potential. The presence of immune checkpoint and stromal interaction markers in AH suggests that the immunologic status of the intraocular environment is reflected in this fluid and may be leveraged for real-time, non-invasive tumor profiling. These insights may inform not only prognosis but also emerging immunotherapeutic strategies, especially given UM's known resistance to systemic checkpoint blockade.

Unlike earlier studies that relied on targeted panels or cytokine profiling, this approach is both comprehensive and data driven. Prior efforts identified risk-associated biomarkers like IL-6 or VEGF but were constrained by hypothesis-driven selection and lacked alignment with GEP classification. In contrast, this model is grounded in direct GEP comparison and built using an unbiased, multi-algorithm selection pipeline, leading to a robust classifier tied explicitly to metastatic risk. The identification of overlapping markers across LASSO, Random Forest, and SVM-RFE underscores the reproducibility and biological relevance of the findings.

The capacity of this model to infer metastatic potential in GEP-unknown cases represents a key advancement over prior work. This provides a practical avenue for risk stratification in patients who cannot undergo tumor biopsy or present with ambiguous clinical features. Furthermore, the instant model's independence from absolute protein quantification enhances its clinical adaptability, as it avoids the complexity and variability associated with calibration across platforms or assay batches.

Integration of AH proteomic profiling with other liquid biopsy modalities, such as circulating tumor DNA (ctDNA), can offer a more holistic assessment of tumor burden and risk. Longitudinal sampling can also reveal dynamic changes in biomarker expression, informing surveillance schedules and early intervention strategies.

In summary, this disclosure marks a significant step toward non-invasive, proteomics-based risk prediction in uveal melanoma. By leveraging machine learning to translate AH protein signatures into clinically actionable insights, a scalable tool is provided doe that can complement or even replace conventional biopsy-based methods—ultimately improving patient management and enabling personalized surveillance strategies.

| Protein | u_statistic | p_value | median_fc |
|---|---|---|---|
| CCL1** | 126 | 0.007115665 | 2.303542836 |
| CCL17** | 126 | 0.007115665 | 2.584880666 |
| CCL19* | 145 | 0.023282417 | 2.728199954 |
| CCL2** | 130 | 0.00925709 | 2.23561178 |
| CCL3* | 153 | 0.036543688 | 2.404883107 |
| CCL7* | 150 | 0.030962508 | 2.517127107 |
| CCL8** | 115 | 0.003324173 | 5.445237295 |
| CXCL10** | 112 | 0.002675243 | 4.691422095 |
| CXCL11* | 114 | 0.003093449 | 6.72564171 |
| CXCL13** | 104 | 0.001468636 | 7.058349548 |
| CXCL8* | 154 | 0.038585243 | 3.177939224 |
| CXCL9** | 128 | 0.008123423 | 4.313033757 |
| FASLG*** | 99 | 0.000994678 | 9.552988576 |
| FLT1* | 132 | 0.010529877 | 2.445331929 |
| GZMA** | 114 | 0.003093449 | 9.079592008 |
| HGF** | 131 | 0.009875228 | 2.495263536 |
| IL15* | 145 | 0.023282417 | 2.183105686 |
| IL17D* | 136 | 0.013550871 | 2.728953571 |
| IL1RN* | 155 | 0.040722923 | 2.705393697 |
| IL6* | 134 | 0.01195603 | 3.351634625 |
| MMP1* | 146 | 0.024670367 | 2.543439781 |
| MMP12* | 149 | 0.029272509 | 5.780665823 |
| OLR1** | 107 | 0.001845721 | 4.769990495 |
| OSM* | 153 | 0.036543688 | 4.583221557 |
| PGF** | 120 | 0.004730437 | 4.955620264 |
| TNFRSF4*** | 91 | 0.000520689 | 4.667055101 |
| TNFRSF9**** | 67 | 6.24E−05 | 15.24506495 |
| VEGFA** | 110 | 0.002309323 | 2.051652209 |

### Example III

### Introduction

Uveal melanoma (UM) is the most common primary intraocular malignancy in adults [1] and carries a high risk of metastasis, primarily to the liver, with up to 50% of patients developing distant disease despite effective local control [2]. Because metastatic UM remains largely incurable, there is a critical need for early, accurate, and minimally invasive tests to guide surveillance and management [3-5]. Current prognostic stratification relies on fine-needle aspiration biopsy (FNAB) for gene expression profiling (GEP) [6] and assessment of the Preferentially Expressed Antigen in Melanoma (PRAME) status [7,8], which together inform metastasis risk. Although effective, FNA requires specialized expertise and carries procedural risks that can limit its use, and in real-world practice, a substantial proportion of UM eyes—including over 30% in our cohort—do not undergo biopsy due to clinical contraindications, tumor characteristics, or patient preference [9-11].

Aqueous humor (AH) biopsy has emerged as a minimally invasive alternative, enabling molecular profiling of the intraocular tumor environment and tumor-associated signals when FNAB is not performed [12]. Proteomics-based liquid biopsy approaches utilizing both vitreous and aqueous humor have demonstrated promise in identifying biomarkers associated with UM progression and metastasis risk [13-15]. However, to date, most AH proteomic studies have relied on relative quantification metrics, such as normalized expression (NPX) values, two-dimensional gel electrophoresis, and novel flowcytometry-based approaches, without estimating absolute protein concentrations in units that support clinical translation [12, 14, 16]. Relative metrics are valuable for discovery, but without calibrated concentration units, they do not establish whether a signal is present at levels compatible with clinically deployable assays, limiting reproducibility, cross-study comparisons, and the development of diagnostic thresholds. Focusing on proteins present above practical detection limits—such as those measurable by standard immunoassays—is essential for advancing clinically deployable biomarkers. Because AH reflects an integrated ocular compartment, this study focuses on identifying tumor-associated signals that reach AH at concentrations compatible with clinical testing. Prior analyses have largely relied on small, predefined cytokine panels, which limit translational applicability by preselecting targets without establishing whether those proteins are present in aqueous humor at concentrations compatible with clinical assay development [15, 17, 18].

To address these gaps, provided herein is the use of proximity extension assays (PEAs) combined with regression-based calibration to estimate absolute protein concentrations (pg/mL) in AH from patients with UM. The goal was to generate a calibrated molecular map of the AH protein targets and characterize how these concentrations relate to established prognostic and clinical features.

### Overview

Uveal melanoma (UM) lacks minimally invasive and reproducible biomarkers to support clinical risk stratification, motivating the need for molecular profiling of aqueous humor (AH) as an alternative to fine-needle tumor aspiration (FNAB). This study aimed to generate a calibrated AH protein concentration map to identify tumor-associated signals present at clinically measurable levels and assess their associations with established molecular and clinical features. AH samples from 70 UM eyes were analyzed using next generation sequencing-based proximity extension assays (PEAs), and leftover AH from 27 samples was further assessed using qPCR-based PEA to obtain reference concentration values. Regression models derived from overlapping proteins enabled extrapolation of calibrated pg/mL-level concentrations across the full cohort. Twenty-three proteins had median modeled concentrations above 5 pg/mL and were examined for clinical relevance and translational feasibility. Several proteins, including CXCL8, CXCL10, VEGFA, HGF, PDCD1, FLT1, FLT3LG, and CCL2, showed progressive increases from GEP1/PRAME− to GEP2/PRAME+ tumors and from AJCC Stage I/II to Stage III/IV, with Stage IV tumors demonstrating significant elevations in CXCL8, VEGFA, and PDCD1. Pathway analysis revealed activation of inflammatory and tumor microenvironment pathways, and upstream regulator analysis identified VEGFA and CCL2 as potential drivers. These findings demonstrate that calibrated AH proteomic profiling can identify clinically measurable protein changes associated with UM risk and stage, supporting its potential utility for biomarker development.

### Materials and Methods

This investigation was a case series study at a tertiary care hospital (Roski Eye Institute, University of Southern California, Los Angeles, CA, USA) conducted with approval by the Institutional Review Board (HS-19-00293). Samples were collected between 2019 and 2024.

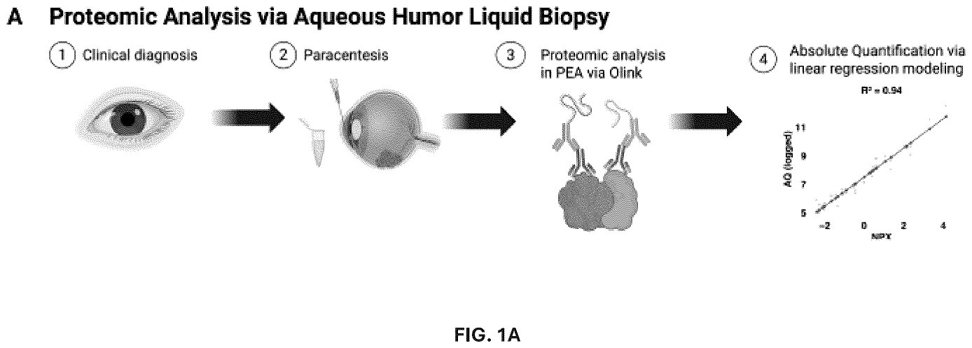

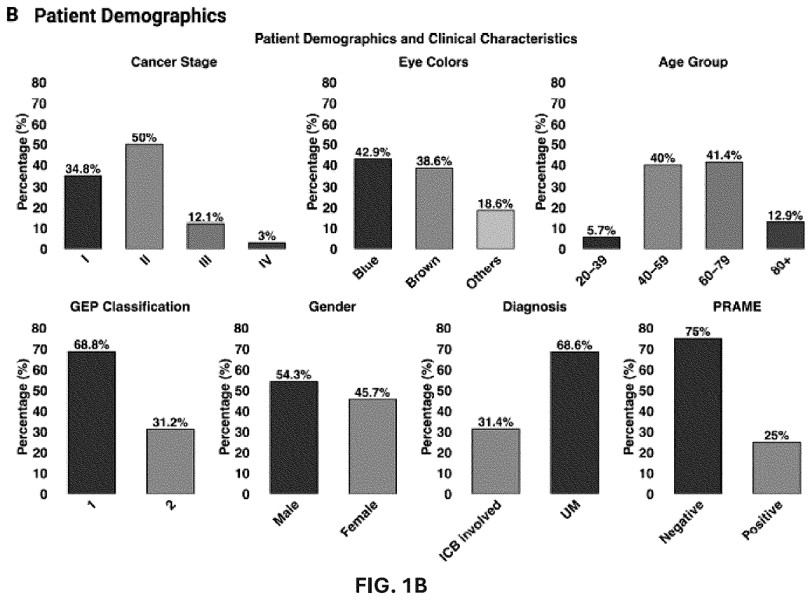

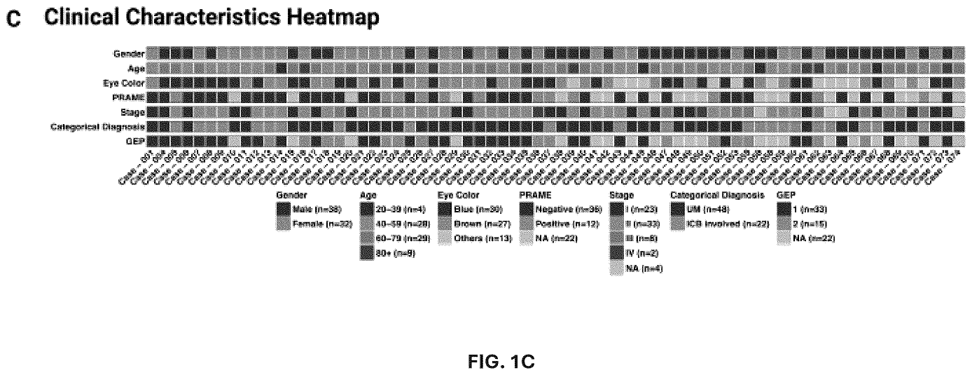

Patient Clinical Characteristics and Demographics This study included a convenience sample of 70 UM patients at the Roski Eye Institute, University of Southern California, from whom written informed consent for an AH sample was obtained. Patient demographics and clinical characteristics are summarized in FIG. 1. All samples consisted of approximately 0.1 mL of AH extracted via clear cornea paracentesis at the end of surgery for brachytherapy plaque placement. 70 AH samples from 70 UM eyes were included. Molecular results were coded and maintained separately from clinical data and thus did not alter patient treatment for all participants.

### Specimen Collection and Storage

A clear corneal paracentesis with a 30-gauge needle was performed to extract ~0.1 mL of AH from UM eyes during clinically indicated surgery to treat UM. The extraction method has been described in detail and published previously by our group for specimen collection from retinoblastoma eyes [19]. Briefly, needles only entered the anterior chamber via the clear cornea at the limbus and did not contact the iris, lens, vitreous, or UM tumor. AH samples were collected intraoperatively but prior to plaque placement or any significant surgical manipulation. Samples were stored on dry ice immediately and transferred to −80 °C within hours for future tests. All samples underwent identical pre-analytic handling and were stored at −80 °C until analysis. Routine FNABs with either a 25- or 27-gauge needle were conducted on 48 patients for mutational analysis and 48 patients for GEP and PRAME status as established clinical tests, which were performed at Castle Biosciences (Phoenix, AZ, USA).

### Proximity Extension Assay (PEA) for Protein Quantification

All aqueous humor samples were collected using a standardized protocol at a single center. Samples were processed uniformly, stored at −80 °C immediately after collection, and underwent no more than one freeze-thaw cycle prior to analysis. Protein expression was initially quantified using next-generation sequencing (NGS)-based PEA, which outputs normalized protein expression (NPX) values. NPX values are unitless, log 2-transformed measures that reflect relative abundance between samples within the same run. While highly reproducible and suitable for comparative analysis, NPX values do not represent absolute protein concentrations and are not directly comparable across experiments or clinical settings.

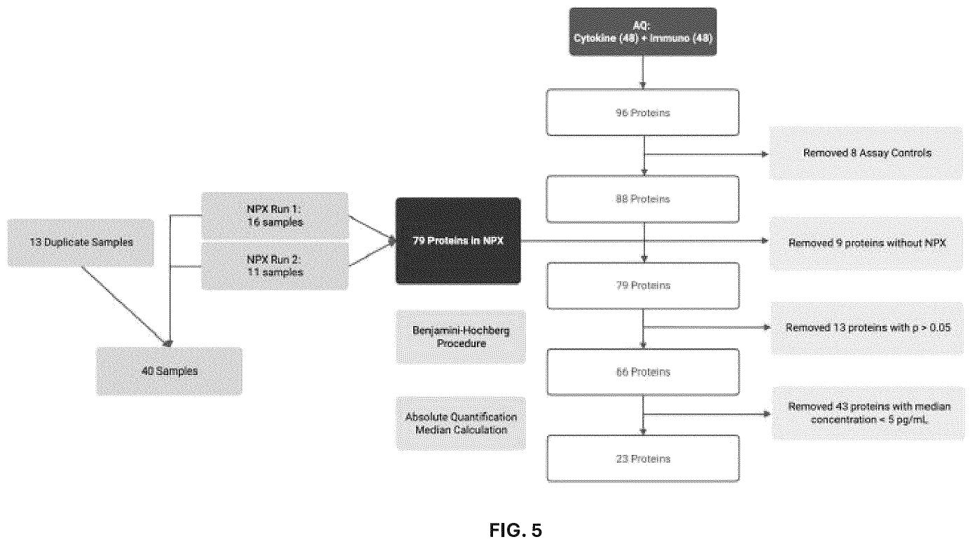

To enable absolute quantification (AQ), leftover sample volume from 27 AH specimens was used for additional analysis via a qPCR-based PEA platform (Olink, Uppsala, Sweden). This platform yields absolute concentrations (pg/mL) for a defined set of targets. FIG. 5 describes the data workflow of these eighty-eight targets. Nine proteins lacked corresponding NPX values in the broader NGS dataset, limiting downstream analysis to 79 proteins. These absolute values served as calibration anchors for extrapolating AQ across the full dataset using regression modeling (FIG. 5). Because absolute concentrations were experimentally measured only in this subset, all reported pg/mL values in the full cohort represent calibrated, regression-modeled estimates rather than direct measurements.

### Regression-Based Extrapolation of Absolute Concentrations

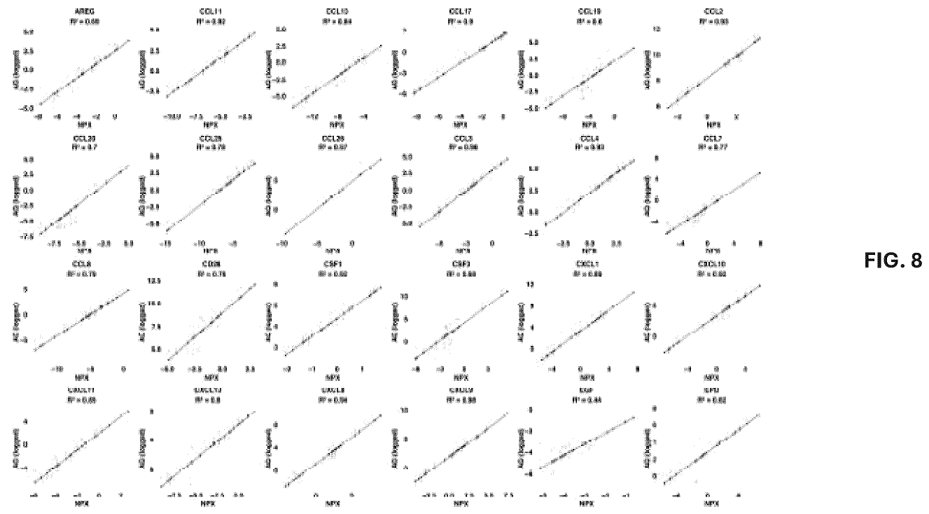

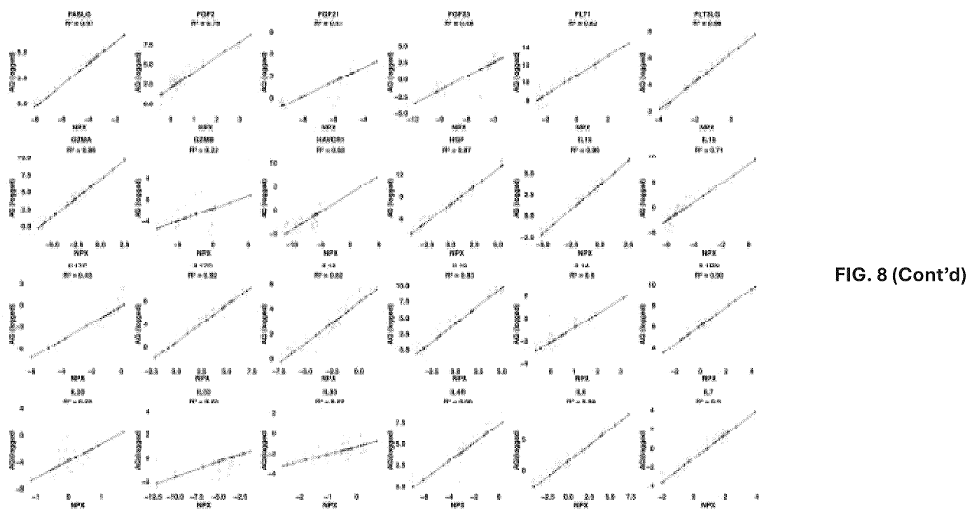

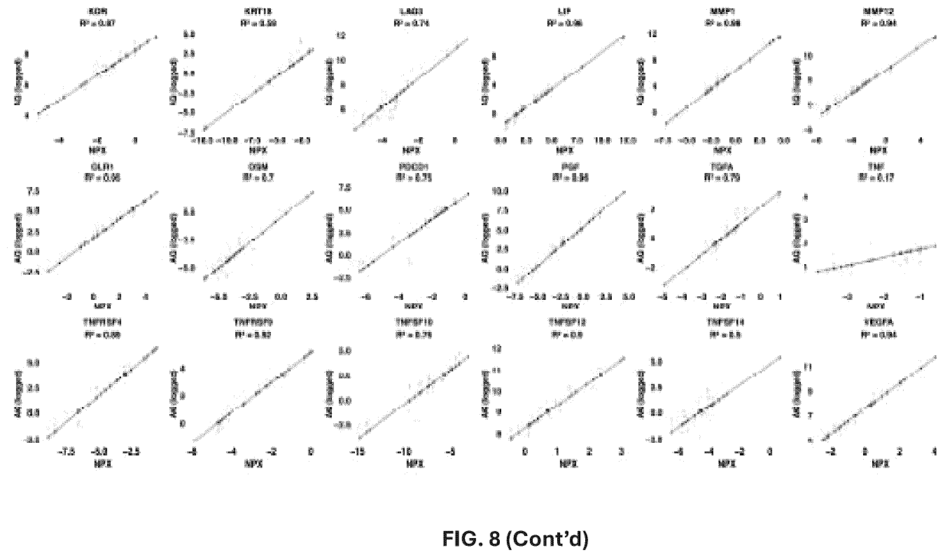

Linear regression models were constructed for 79 calibration proteins to correlate NPX values from high-throughput PEA with absolute concentrations derived from qPCR-based analysis. The 27 qPCR-assayed samples, including 13 technical duplicates, yielded 40 paired measurements used to construct the calibration models. These models enabled extrapolation of absolute protein concentrations (pg/mL) across the full 70-sample cohort. Inclusion of technical replicates allowed our models to be estimated using real-world variations in NPX values that can be observed across multiple NGS runs from the same source. Because no bridging samples were originally included across the two NGS runs, an additional targeted Olink qPCR-based run was performed that included 40 samples spanning the NPX1-qPCR (2021) and NPX2-qPCR (2023) datasets. This bridging run allowed us to align and correct for batch effects between the two measurement panels. Batch effects were corrected using linear adjustments (median difference between bridging samples analyzed across platforms) and by including batch as a covariate in models. Regression performance showed strong linearity across most proteins (see FIG. 8 for R2 values and model diagnostics).

### Gene Ontology and Pathway Analysis

Functional annotation of protein expression data was performed using QIAGEN's IngenuityR Pathway Analysis (IPAR, QIAGEN, Hilden, Germany; web-based version: www.qiagen.com/ingenuity, accessed on 20 May 2025). A total of 23 proteins differentially expressed between early-stage (Stages I+II, n=56) and advanced-stage (Stages III+IV, n=10) cases were subjected to canonical pathway and upstream regulator analysis (URA). Protein-level fold changes and statistical significance were assessed using the Mann-Whitney U test, and results were uploaded to IPA for downstream interpretation. IPA generated activation z-scores and corresponding p-values for each pathway or regulator. The z-score predicts the activation state of a pathway or regulator, with positive values indicating activation and negative values indicating inhibition. Pathways or regulators with an absolute z-score≥2 were considered significantly activated or inhibited.

### Statistical Analysis

All statistical analyses were performed in R version 4.4.1 (R Core Team, 2024). Linear regression models were constructed using the lm( ) function, with custom scripts for normalization and extrapolation. These models integrated NPX values from two independent NGS-based PEA runs with absolute quantification data obtained from qPCR-based PEA (79 proteins; 40 paired data points across 27 samples, including 13 technical duplicates), enabling extrapolation of absolute protein concentrations (pg/mL) across the full cohort of 70 UM samples. To correct for any heteroscedasticity in model residuals, Huber-White robust standard error terms were used. Multiple testing correction was performed using the Benjamini-Hochberg False Discovery Rate procedure, resulting in 66 proteins retained for downstream analysis (FIG. 5). Overlapping samples across the qPCR runs provided internal cross-validation and supported consistency of protein measurements across platforms.

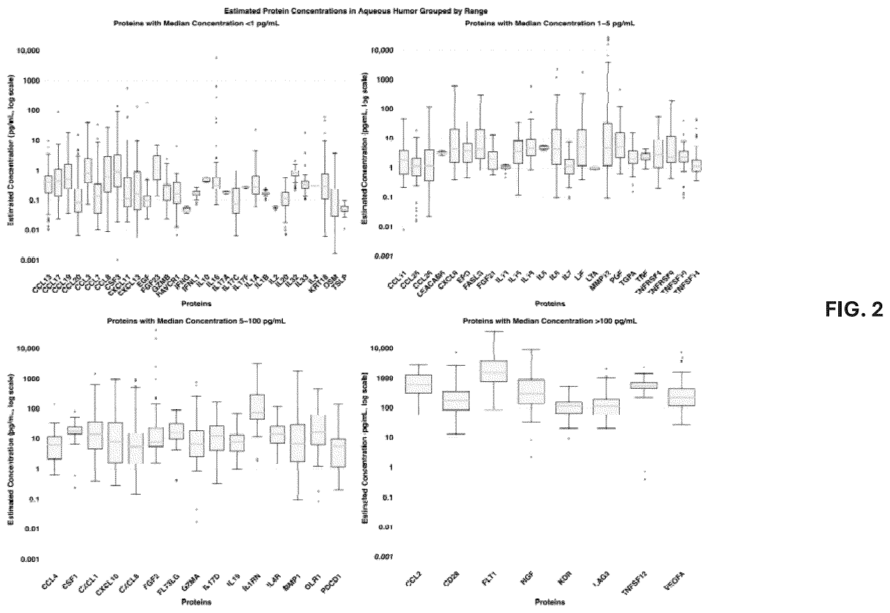

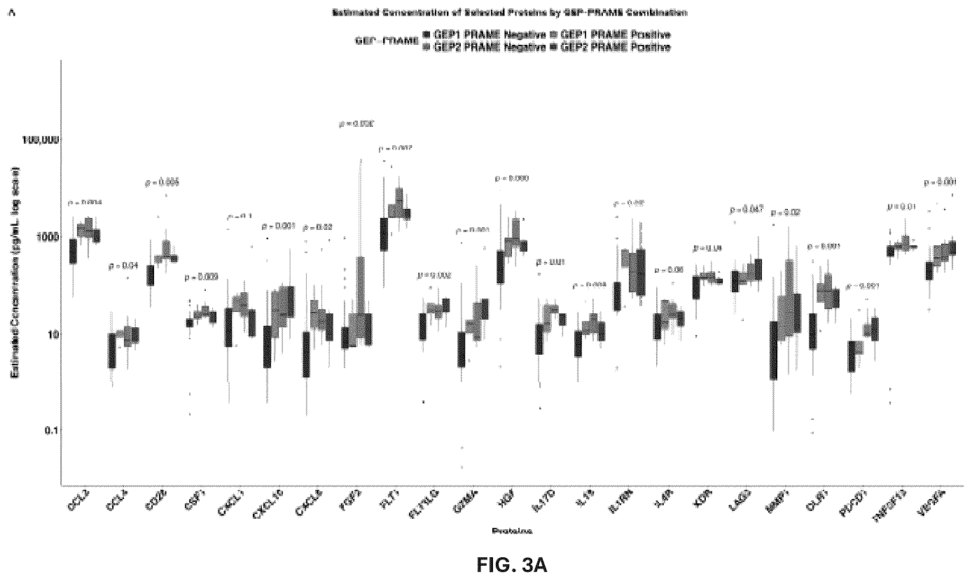

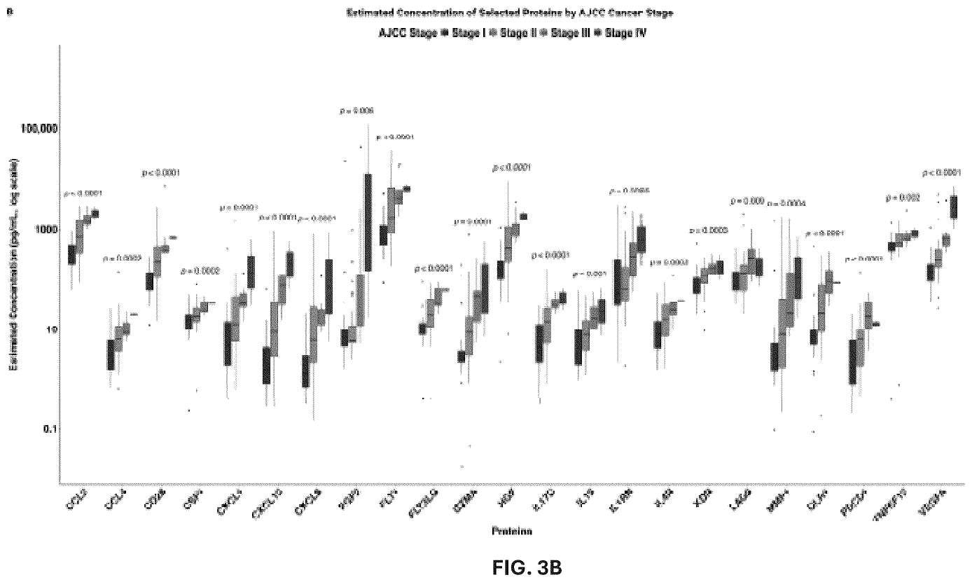

Of the 66 proteins, 43 had median concentrations below 5 pg/mL and were excluded from further analysis due to low abundance, which limits the feasibility of developing analytically robust assays at this concentration range. Differential protein expression was assessed using rank-based statistical tests for each protein when comparing patient groups stratified by GEP/PRAME status or clinical stage (FIG. 3A,3B). Untransformed protein concentrations were reported in the figures to preserve interpretability, alongside p-values derived from ranked data for more robust significance testing. To assess the robustness of regression modeled concentrations, a simulation-based sensitivity analysis using prediction intervals was performed; results based on modeled data were compared to n=100 datasets of values that were randomly adjusted to other values within the prediction interval. Data visualization was conducted in R, with boxplots used to illustrate expression distributions across groups. All statistical tests were two-sided, with significance defined as FDR adjusted p<0.05 unless otherwise indicated.

### Results

### Clinical Demographics

A total of 70 AH samples were collected from 70 diagnostic UM eyes at the time of plaque brachytherapy placement. Of these, GEP of tumor FNAB classified 33 samples as GEP1 (low metastatic risk) and 15 samples as GEP2 (high metastatic risk). Biopsies were not performed in 22 cases. Demographic and clinical characteristics of the cohort are shown in FIG. 1. The cohort included 38 male (54.3%) and 32 female (45.7%) patients, with a median age at diagnosis of 61 years (range: 22-97 years). Uveal melanoma with choroidal involvement was present in 48 eyes (68.6%), while 22 eyes exhibited uveal melanoma involving the iris or ciliary body (ICB) (31.4%). Among the eyes that underwent biopsy, 75% of eyes were PRAME-negative, while 25% were PRAME-positive. Eye color distribution showed a predominance of blue eyes (42.9%), followed by brown (38.6%) and others (18.6%).

### Generating Calibrated Protein Concentration Estimates

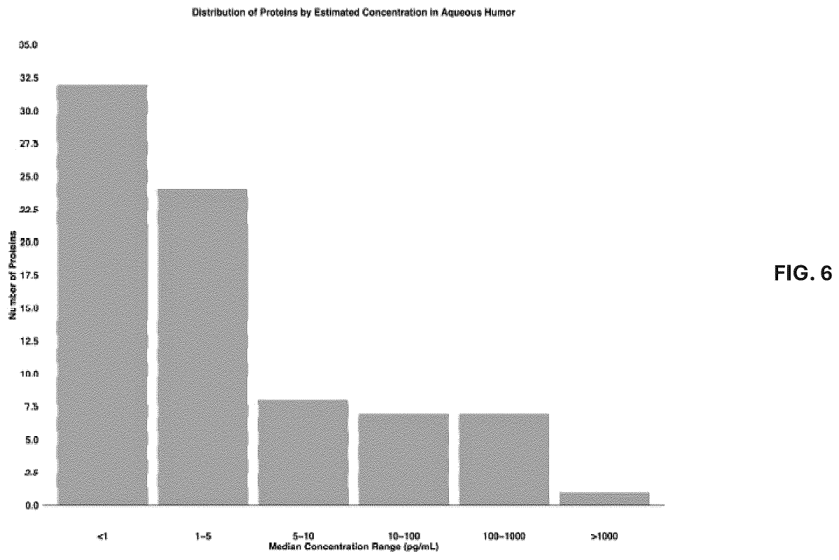

Using a dual-platform proximity extension assay (PEA), we generated calibrated protein concentration estimates (pg/mL) across 79 proteins in AH from the full 70-sample cohort (FIG. 5). Protein concentrations ranged widely, from sub-picogram levels to nanograms per milliliter (ng/mL). The distribution of absolute concentrations for 79 proteins across all aqueous humor samples is listed (FIG. 6). Among them, 23 proteins exhibited median calibrated concentrations above the conservative detection threshold of 5 pg/mL (FIG. 2). This threshold reflects the low detection limits typical of standard immunoassays. The majority of proteins were found in the lower concentration ranges, with the highest frequency observed in the <1 pg/mL and 1-5 pg/mL ranges. A single protein, FLT1, exhibited a median estimated concentration >1000 pg/mL, distinguishing it as the most abundant among the profiled analytes (FIG. 2).

These 23 proteins with modeled median concentrations above 5 pg/mL were identified as abundant proteins in AH. The concentrations of these 23 proteins were compared among four molecular subgroups defined by GEP class (Class 1 vs. Class 2) and PRAME status (negative vs. positive; FIG. 3A). Several proteins demonstrated a clear trend across prognostic categories, particularly for: GZMA, CXCL10, FLT3LG, CD28, VEGFA, HGF, and TNFSF12 (FIG. 3A). As expected, cases with GEP1 and PRAME negativity (n=28) had the lowest concentration of these proteins, correlating with improved prognosis and less severity of disease. These comparisons reflect cross-sectional associations and not longitudinal progression.

The calibrated concentrations of these proteins above the detection threshold was evaluated across AJCC clinical stages (FIG. 3B; Stages I to IV). Higher concentrations were observed in later-stage tumors for multiple proteins, including CXCL8, CXCL10, CCL4, GZMA, MMP1, IL17D, CXCL1, IL19, IL4R, FLT3LG, CSF1, IL1RN, KDR, CD28, VEGFA, HGF, CCL2, TNFSF12, and FLT1, with generally increasing levels from minor (Stages I/II) to advanced (Stages III/IV) stages.

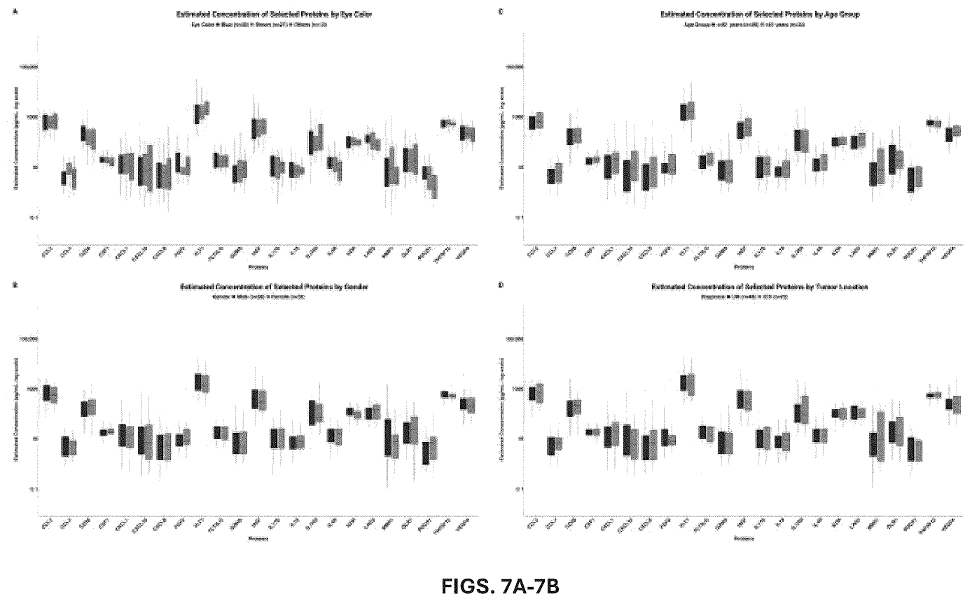

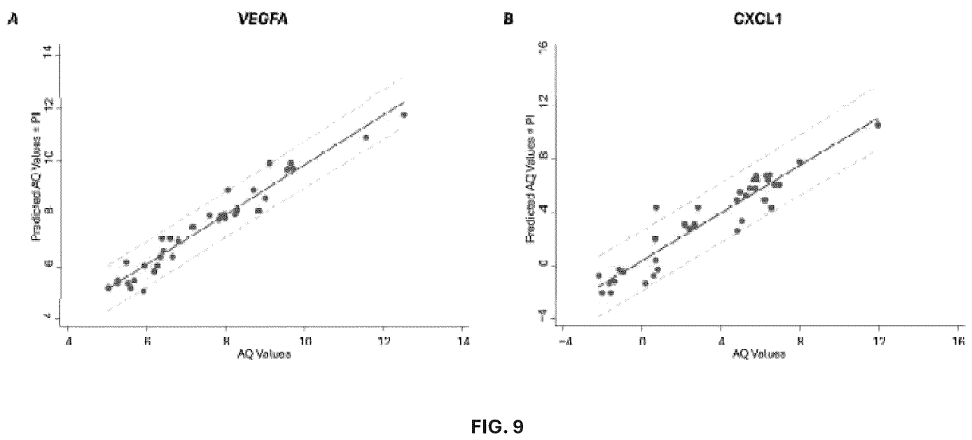

Several proteins showed statistically significant increases in concentrations across advancing AJCC stages, including CXCL8 (p<0.0001), VEGFA (p<0.0001), and PDCD1 (p<0.0001). Stage IV tumors (n=2) showed higher median concentrations for these proteins relative to earlier stages. These patterns are consistent with enrichment of proinflammatory or immunoregulatory factors in more advanced disease (FIG. 3B). Nonsignificant differences were observed when comparing eye color, age, tumor location, and gender (FIG. 7). All significance values reflect rank-based tests with modeled concentration inputs. Key stage-associated proteins retained statistical significance across repeated simulations incorporating model prediction intervals, supporting robustness of the modeled estimates (FIG. 9).

### Pathway Analysis

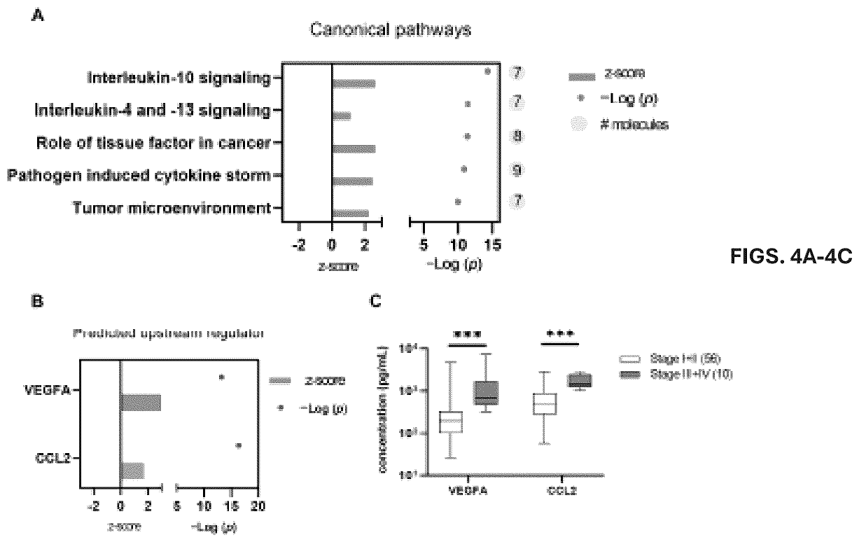

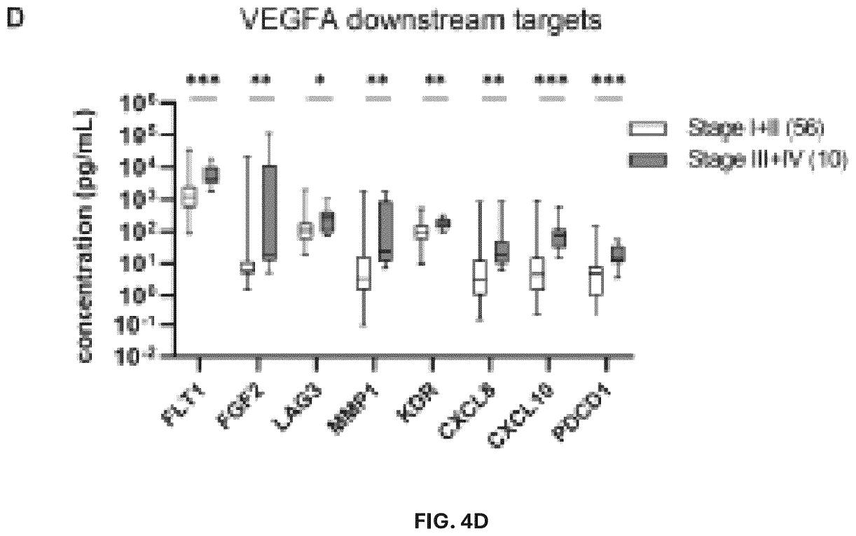

To determine the biological relevance of the 23 proteins above the detection threshold, pathway analysis was conducted to identify the important cellular functions these DEPs may provide. After subjecting the 23 DEPs to Qiagen Ingenuity Pathway Analysis (IPA), the canonical pathway analysis revealed significant enrichment in immune and tumor-related pathways, with the top hits including Interleukin-10 signaling, pathogen-induced cytokine storm, and tumor microenvironment. These pathways exhibited positive activation z-scores, consistent with potential activation in advanced-stage tumors (FIG. 4A; z-score>2).

An upstream regulator analysis (URA) was performed to identify the potential upstream target that has been experimentally verified to affect the expression of the 23 DEPs. URA identified VEGFA (z=3.1) and CCL2 (z=1.7) as the strongest predicted upstream drivers (FIG. 4B). Both exhibited higher expression in advanced disease stages (III-IV) compared to early stages (I-II) in AH (FIG. 4C). Evaluation of VEGFA downstream targets (FLT1, FGF2, LAG3, MMP1, KDR, CXCL8, CXCL10, and PDCD1) showed significantly higher concentrations in advanced-stage tumors, consistent with increased activity of the VEGFA-associated signaling axis in advanced disease (FIG. 4D).

### Discussion

This study presents the first broad-scale calibrated estimation of AH proteins in UM. The findings demonstrate that AH proteomics can serve as a minimally invasive technique that offers greater insight into clinical characteristics of uveal melanoma, offering a safer and repeatable platform for risk stratification. Previous AH proteomics studies have primarily relied on relative expression metrics, such as NPX values, which are suitable for discovery but limited in reproducibility across cohorts and in defining clinical thresholds. By generating calibrated pg/mL estimates based on regression models, standardized measurement was enabled across patients and cohorts, supporting the development of reproducible diagnostic thresholds and assays, and distinguishing proteins that are realistically measurable using conventional clinical assays.

Statistical analysis confirmed the significance of these biomarkers in distinguishing UM clinical features. The activation of immune- and tumor-related pathways in advanced-stage tumors points to a coordinated inflammatory and angiogenic response during disease progression. VEGFA and CCL2 emerged as central upstream regulators, consistent with influencing the observed proteomic shifts through vascular remodeling and immune modulation.

VEGFA, a key pro-angiogenic factor, drives tumor vascularization and may facilitate metastatic spread. It has long been established in uveal melanoma that elevated aqueous VEGFA levels correlate with tumor size, disease progression, and patient survival [20]. Hypoxia within the tumor microenvironment induces VEGFA expression via the HIF-1a pathway, suggesting that VEGFA may contribute to tumor vascularization and could serve as a therapeutic target [21]. FLT1 encodes VEGFR-1, a receptor for VEGFA, and plays a role in angiogenesis. While specific studies on FLT1 in UM are limited, its interaction with VEGFA suggests that FLT1 may be involved in tumor vascularization and progression. Targeting the VEGFA-FLT1 axis could be a potential therapeutic strategy in UM. The consistent upregulation of VEGFA downstream effectors reinforces its pivotal role, consistent with greater activity of the VEGFA-associated pathway during UM progression.

In UM, the chemokine CCL2 is produced by tumor cells and monocytes, attracting tumor-associated macrophages that can promote tumor growth and suppress immune response. Its expression is associated with chromosome 3 loss, a marker of poor prognosis in UM [17]. Elevated CCL2 levels have been reported in patients with melanoma and are associated with tumor metastasis, immunosuppression, and disease progression, as it mediates the invasion and growth of metastatic melanoma. The involvement of the CCR2/CCL2 axis is of particular importance in the early stages of tumor invasion and is regarded as a predictor of poor prognosis in cancer patients [22]. Additionally, CXCL10 facilitates the recruitment of immune cells and directly promotes tumor vascularization and growth, upregulating pro-angiogenic factors such as VEGF, PDGF-B, and FGF2 [23]. The associations observed in the AH dataset are consistent with these described mechanisms, although causal or cellular source inferences cannot be inferred from this cross-sectional analysis.

Collectively, these findings highlight distinct protein expression patterns in AH associated with both molecular features (GEP and PRAME status) and clinical progression, demonstrating utility for stratifying disease aggressiveness in uveal melanoma. All protein concentrations represent regression-modeled estimates derived from measured reference values. To address volume limitations in AH samples and enable calibrated estimation across the full cohort, a calibration approach was applied based on regression modeling (FIG. 8). Reference concentrations were experimentally measured in a subset of samples and then extrapolated across the full dataset using normalized NPX values. This method allowed one to derive pg/mL values across 66 proteins while correcting for batch effects and inter-run variability. By reporting calibrated concentrations rather than relative metrics, the analysis improves reproducibility, supports cross-study benchmarking, and enables diagnostic assay development. Simulation-based sensitivity analyses incorporating prediction intervals indicated that the strongest stage-associated signals were robust to re-introduction of modeled variance, supporting the reliability of the regression-based concentration estimates (FIG. 9).

Compared with tears, which are influenced by environmental exposure, lacrimal physiology, and ocular surface inflammation, AH more directly reflects intraocular tumor-associated signaling. Systemic biofluids such as plasma may introduce additional confounding from systemic inflammatory or oncologic processes and may dilute tumor-derived signals below detection thresholds [26,27]. Accordingly, while distal biofluids hold translational promise, AH currently provides the most proximate and biologically interpretable fluid compartment for characterizing the uveal melanoma microenvironment.

### BIBLIOGRAPHY

1. Jager, M. J. et al. Uveal Melanoma. Nat. Rev. Dis. Primers 2020, 6, 24. Correction in Nat. Rev. Dis. Primers 2022, 8, 4.
2. Rodriguez-Vidal, et al. Treatment of Metastatic Uveal Melanoma: Systematic Review. Cancers 2020, 12, 2557.
3. Beasley, A. B. et al. Future Perspectives of Uveal Melanoma Blood Based Biomarkers. Br. J. Cancer 2022, 126, 1511-1528.
4. Lamas, N.J. et al. Prognostic Biomarkers in Uveal Melanoma: The Status Quo, Recent Advances and Future Directions. Cancers 2021, 14, 96.
5. Yesiltas, Y. S. et al. Surveillance for Metastasis in High-Risk Uveal Melanoma Patients: Standard Versus Enhanced Protocols. Cancers 2023, 15, 5025.
6. Onken, M. D. et al. Collaborative Ocular Oncology Group Report Number 1: Prospective Validation of a Multi-Gene Prognostic Assay in Uveal Melanoma. Ophthalmology 2012, 119, 1596-1603.
7. Field, M. G. et al. Epigenetic Reprogramming and Aberrant Expression of PRAME Are Associated with Increased Metastatic Risk in Class 1 and Class 2 Uveal Melanomas. Oncotarget 2016, 7, 59209-59219.
8. Field, M. G. et al. PRAME as an Independent Biomarker for Metastasis in Uveal Melanoma. Clin. Cancer Res. 2016, 22, 1234-1242.
9. Eide, N.; Walaas, L. Fine-Needle Aspiration Biopsy and Other Biopsies in Suspected Intraocular Malignant Disease: A Review. Acta Ophthalmol. 2009, 87, 588-601.
10. Karcioglu, Z. A.; Gordon, R. A.; Karcioglu, G. L. Tumor Seeding in Ocular Fine Needle Aspiration Biopsy. Ophthalmology 1985, 92, 1763-1767.
11. Singh, A. D.; Biscotti, C. V. Fine Needle Aspiration Biopsy of Ophthalmic Tumors. Saudi J. Ophthalmol. 2012, 26, 117-123.
12. Wolf, J. et al. Liquid-Biopsy Proteomics Combined with AI Identifies Cellular Drivers of Eye Aging and Disease In Vivo. Cell 2023, 186, 4868-4884.e12.
13. Heiferman, M. J.; Mahajan, V. B.; Mruthyunjaya, P. Proteomics in Uveal Melanoma. Curr. Opin. Ophthalmol. 2022, 33, 202-210.
14. Velez, G. et al. Liquid Biopsy Proteomics of Uveal Melanoma Reveals Biomarkers Associated with Metastatic Risk. Mol. Cancer 2021, 20, 39.
15. Cheng, Y.; Feng, J.; Zhu, X.; Liang, J. Cytokines Concentrations in Aqueous Humor of Eyes with Uveal Melanoma. Medicine 2019, 98, e14030.
16. Zuidervaart, W. et al. Proteomic Analysis of Uveal Melanoma Reveals Novel Potential Markers Involved in Tumor Progression. Investig. Ophthalmol. Vis. Sci. 2006, 47, 786-793.
17. Midena, E. et al. In Vivo Intraocular Biomarkers. Medicine 2020, 99, e22091.
18. Demirci, H. et al. Investigating Vitreous Cytokines in Choroidal Melanoma. Cancers 2023, 15, 3701.
19. Berry, J. L. et al. Genomic cfDNA Analysis of Aqueous Humor in Retinoblastoma Predicts Eye Salvage: The Surrogate Tumor Biopsy for Retinoblastoma. Mol. Cancer Res. 2018, 16, 1701-1712.
20. Missotten, G. S. O. et al. Vascular Endothelial Growth Factor A in Eyes with Uveal Melanoma. Arch. Ophthalmol. 2006, 124, 1428-1434.
21. el Filali, M. et al. Regulation of VEGF-A in Uveal Melanoma. Investig. Ophthalmol. Vis. Sci. 2010, 51, 2329-2337.
22. Basson, C. et al. Chemokines as Possible Therapeutic Targets in Metastatic Melanoma. Cancer Med. 2023, 12, 14387-14402
23. Leonard-Murali, S. et al. Uveal Melanoma Immunogenomics Predict Immunotherapy Resistance and Susceptibility. Nat. Commun. 2024, 15, 2863.
24. Moshtaghion, S. M. M. et al. VEGF in Tears as a Biomarker for Exudative Age-Related Macular Degeneration: Molecular Dynamics in a Mouse Model and Human Samples. Int. J. Mol. Sci. 2025, 26, 3855.
25. Yoon, K.-C. et al. Expression of CXCL9, -10, -11, and CXCR3 in the Tear Film and Ocular Surface of Patients with Dry Eye Syndrome. Investig. Ophthalmol. Vis. Sci. 2010, 51, 643-650.
26. Berry, J. L. et al. Aqueous Humor Is Superior to Blood as a Liquid Biopsy for Retinoblastoma. Ophthalmology 2020, 127, 552-554.
27. Walz, J. M. et al. Pre-Analytical Parameters Affecting Vascular Endothelial Growth Factor Measurement in Plasma: Identifying Confounders. PLoS ONE 2016, 11, e0145375.

### Example IV—An 18-Protein Aqueous Humor Classifier Derived from a Clinically Deployable Multiplex Platform Predicts Metastatic Risk in Uveal Melanoma Including Eyes Without Tumor Biopsy

### Patient Cohort and Sample Classification

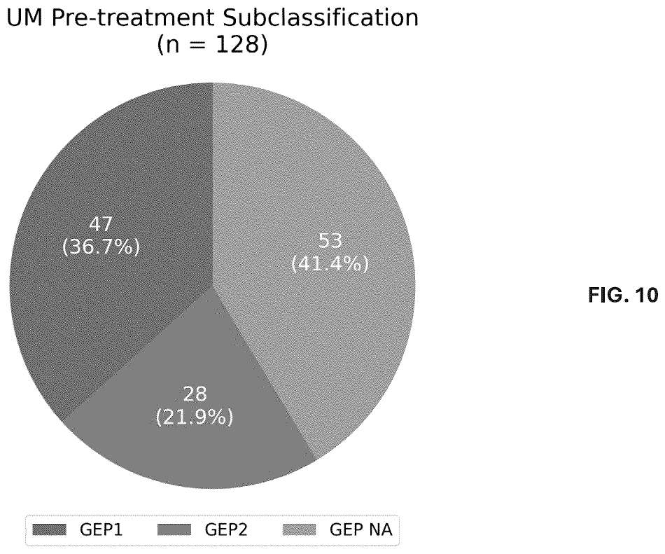

A total of 128 aqueous humor (AH) samples were collected from uveal melanoma (UM) patients prior to any therapeutic intervention. The cohort was subclassified based on the availability of tumor-derived gene expression profile (GEP) data. Of the 128 samples, 47 were classified as GEP1 (low metastatic risk), representing 36.7% of the cohort; 28 were classified as GEP2 (high metastatic risk), representing 21.9% of the cohort; and 53 samples lacked tumor-derived GEP data and were designated GEP-unknown (GEP NA), representing 41.4% of the cohort. The distribution of GEP classifications across the cohort is illustrated in FIG. 10. The proportion of GEP-unknown samples in this cohort is consistent with reported rates of biopsy non-performance in real-world uveal melanoma practice, wherein anatomic, clinical, or patient-related factors preclude fine-needle aspiration biopsy in a substantial fraction of patients.

Clinical characteristics of the cohort stratified by GEP classification and by protein expression profile (PEP) classification generated by the disclosed classifier are presented in Table 1. Among GEP-classified patients, GEP1 (n=47) and GEP2 (n=28) groups did not differ significantly with respect to sex (p=0.234), laterality (p=0.242), age at diagnosis (63.98±18.83 versus 66.5±11.56 years; p=0.685), eye color (p=0.441), or tumor location (p=0.442). Statistically significant differences were observed between GEP1 and GEP2 groups in AJCC stage distribution (p=0.002) and tumor volume (436.2±548.8 mm³ versus 603.7±514.8 mm³; p=0.024), consistent with the known association between GEP2 classification and more advanced disease. Among GEP1 patients, 23.4% presented at AJCC Stage I and 63.8% at Stage II, while among GEP2 patients, only 7.1% presented at Stage I and 28.6% presented at Stage III. Similarly, among patients classified by the aqueous humor-based protein expression profile classifier as PEP1 (n=41) or PEP2 (n=12), statistically significant differences were observed in AJCC stage (p=0.004) and tumor volume (85.18±100.3 mm³ versus 356.5±385.6 mm³; p=0.013), while sex, laterality, age, eye color, and tumor location did not differ significantly between PEP groups. Table 1.

TABLE 1
Clinical characteristics of [symbol] cohorts [symbol] by gene
expression profile (GEP) and protein expression profile (PEP)

| | GEP1 | GEP2 | P | PEP1 | PEP2 | P |
|---|---|---|---|---|---|---|
| | (n = | (n = | | (n = | (n = | |
| Variable | [symbol] ) | [symbol] ) | value | [symbol] ) | [symbol] ) | value |
| Sex | | | [symbol] | | | [symbol] |
| Female | [symbol] | [symbol] | | [symbol] | [symbol] | |
| Male | [symbol] | [symbol] | | [symbol] | [symbol] | |
| [symbol] | | | [symbol] | | | [symbol] |
| Right (OD) | [symbol] | [symbol] | | [symbol] | [symbol] | |
| Left (OS) | [symbol] | [symbol] | | [symbol] | [symbol] | |
| Both (OU) | [symbol] | [symbol] | | [symbol] | [symbol] | |
| Age at | [symbol] | [symbol] | [symbol] | [symbol] | [symbol] | [symbol] |
| diagnostic years | | | | | | |
| Eye colour | | | [symbol] | | | [symbol] |
| Light | [symbol] | [symbol] | | [symbol] | [symbol] | |
| Dark | [symbol] | [symbol] | | [symbol] | [symbol] | |
| Tu [symbol] location | | | [symbol] | | | [symbol] |
| Anterior | [symbol] | [symbol] | | [symbol] | [symbol] | |
| Posterior | [symbol] | [symbol] | | [symbol] | [symbol] | |
| AJCC stage | | | [symbol] | | | [symbol] |
| I | [symbol] | [symbol] | | [symbol] | [symbol] | |
| II | [symbol] | [symbol] | | [symbol] | [symbol] | |
| III | [symbol] | [symbol] | | [symbol] | [symbol] | |
| IV | [symbol] | [symbol] | | [symbol] | [symbol] | |
| Tu [symbol] volume | [symbol] | [symbol] | [symbol] | [symbol] | [symbol] | [symbol] |

[symbol]

indicates data missing or illegible when filed

### Development of the 18-Protein Classifier

To develop a clinically deployable classifier compatible with a targeted multiplex proteomic platform, an 18-protein panel was selected from proteins detectable using the Olink Flex assay system. Protein expression levels were quantified in AH samples as normalized protein expression (NPX) values on a log 2 scale using the proximity extension assay (PEA) platform. The 18 proteins comprising this panel are: TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.

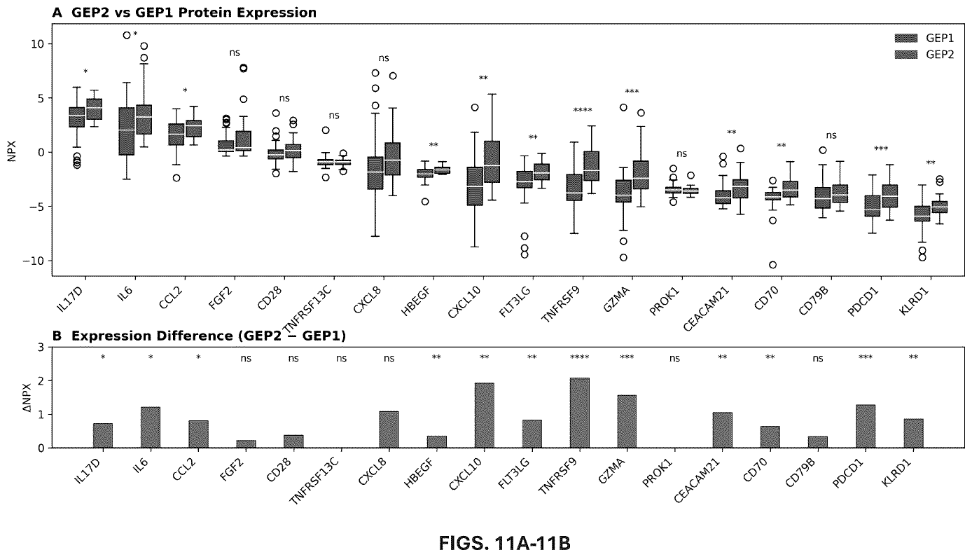

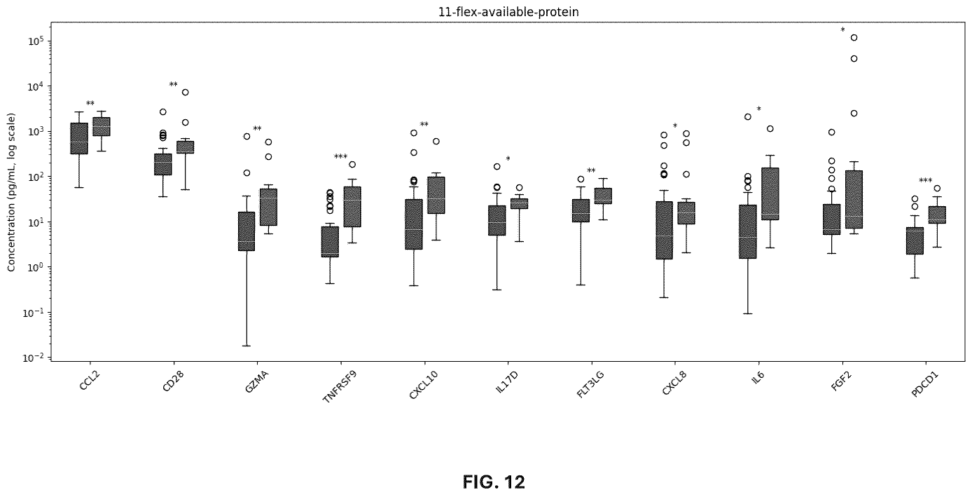

NPX expression levels of the 18 panel proteins were compared between GEP1 and GEP2 samples. As shown in FIG. 11A, several proteins demonstrated statistically significant differential expression between GEP1 and GEP2 groups. Proteins including TNFRSF9 (p<0.0001), GZMA (p<0.001), CXCL10 (p<0.01), FLT3LG (p<0.01), PDCD1 (p<0.001), KLRD1 (p<0.01), and HBEGF (p<0.01) showed higher NPX values in GEP2 samples relative to GEP1 samples. FIG. 11B presents the expression difference (delta NPX, GEP2 minus GEP1) for each of the 18 proteins, with TNFRSF9 and HBEGF demonstrating the largest magnitude differences between risk groups.

### Absolute Concentration Estimation for Clinically Deployable Protein Subset

To assess the translational feasibility of the 18-protein panel, absolute protein concentrations were estimated for 11 of the 18 panel proteins using regression-based calibration from paired qPCR-based PEA measurements, as described herein. Estimated concentrations for CCL2, CD28, GZMA, TNFRSF9, CXCL10, IL17D, FLT3LG, CXCL8, IL6, FGF2, and PDCD1 are presented in FIG. 12 on a logarithmic scale (pg/mL). Several proteins demonstrated statistically significant concentration differences between GEP1 and GEP2 groups at the concentration level, including GZMA (p<0.001), TNFRSF9 (p<0.001), CXCL10 (p<0.01), FLT3LG (p<0.01), and PDCD1 (p<0.001). The majority of proteins in this subset exhibited median estimated concentrations above the 5 pg/mL detection threshold used as a criterion for assay development feasibility, supporting the practical deployability of this panel in a clinical assay context.

### Classifier Performance in GEP-Classified Samples

A logistic regression classifier was trained on the 75 GEP-classified AH samples (47 GEP1 and 28 GEP2) using the 18-protein panel. The trained classifier was expressed as:

logit(p)=−5.5935+1.5499(TNFRSF9)+2.6571(CD70)−3.7116(TNFRSF13C)−5.3780(PROK1)+1.5212(CEACAM21)−1.1170(PDCD1)+1.0400(GZMA)+1.6626(CCL2)−1.3980(IL6)+0.2725(FGF2)+0.8791(CXCL10)−0.8435(CXCL8)+0.9193(IL17D)+1.1831(KLRD1)−0.9768(CD28)−2.1695(CD79B)−0.3674(FLT3LG)+2.9745(HBEGF)

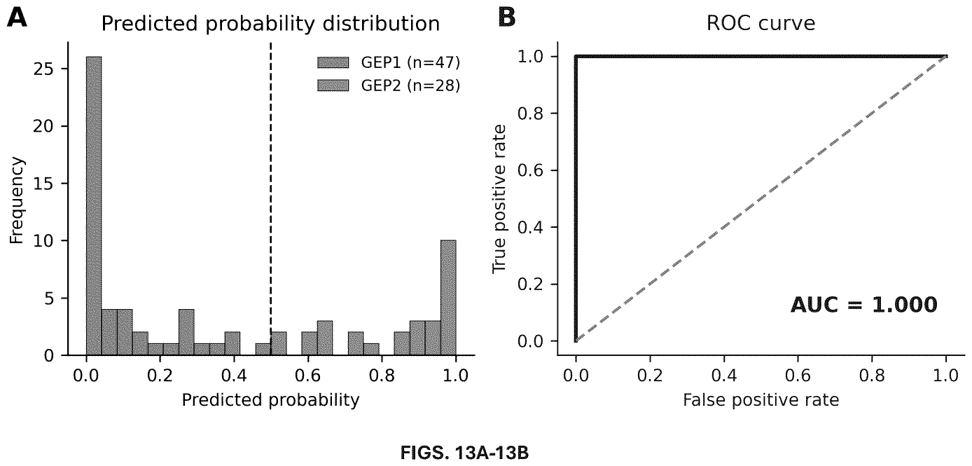

Classifier performance was evaluated by receiver operating characteristic (ROC) curve analysis and assessment of predicted probability distributions across GEP classes. As shown in FIG. 13A, the predicted probability distribution demonstrated clear separation between GEP1 and GEP2 samples at a classification threshold of 0.50, with the majority of GEP1 samples receiving predicted probabilities below 0.1 and the majority of GEP2 samples receiving predicted probabilities above 0.5. ROC curve analysis yielded an area under the curve (AUC) of 1.000, indicating complete separation of GEP1 and GEP2 samples by the 18-protein classifier in the training cohort (FIG. 13B).

### Application of the 18-Protein Classifier to GEP-Unknown Samples

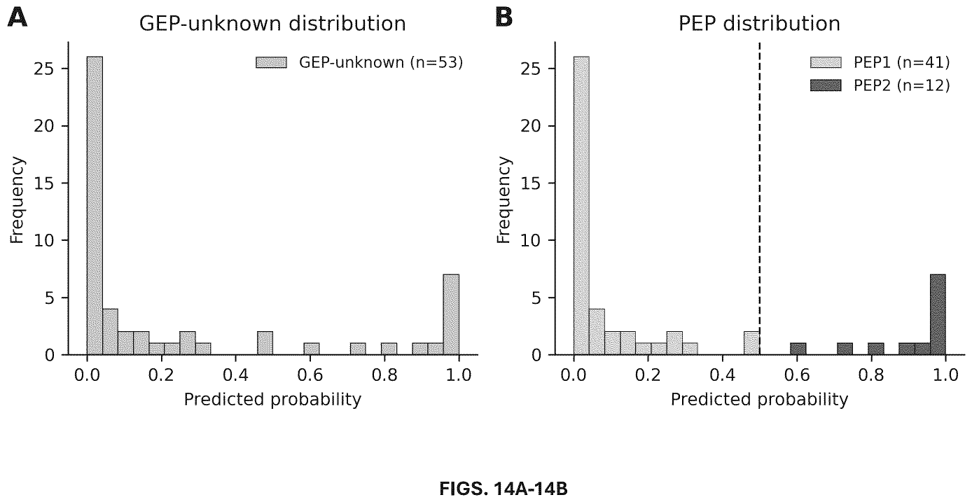

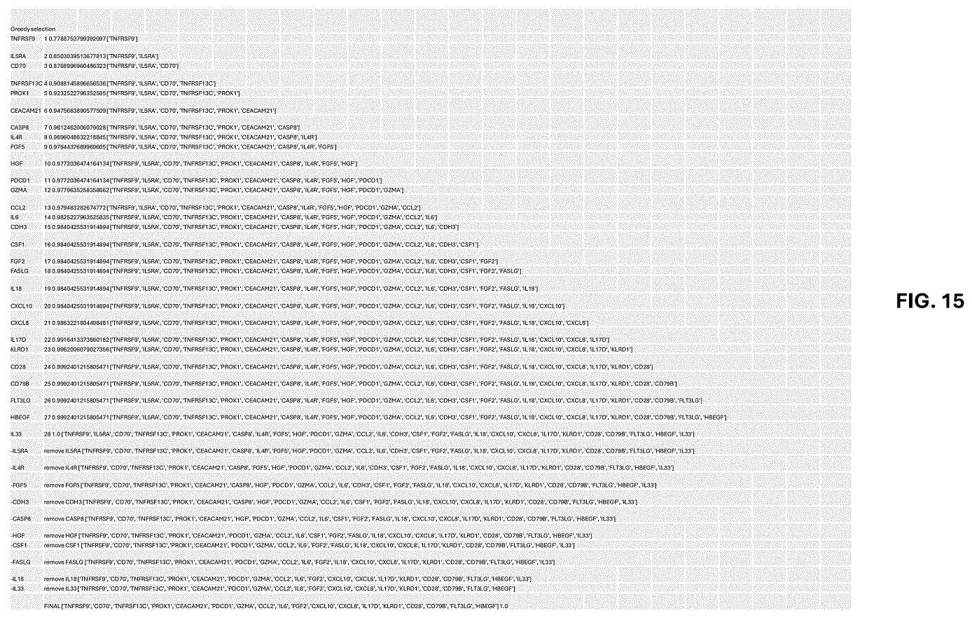

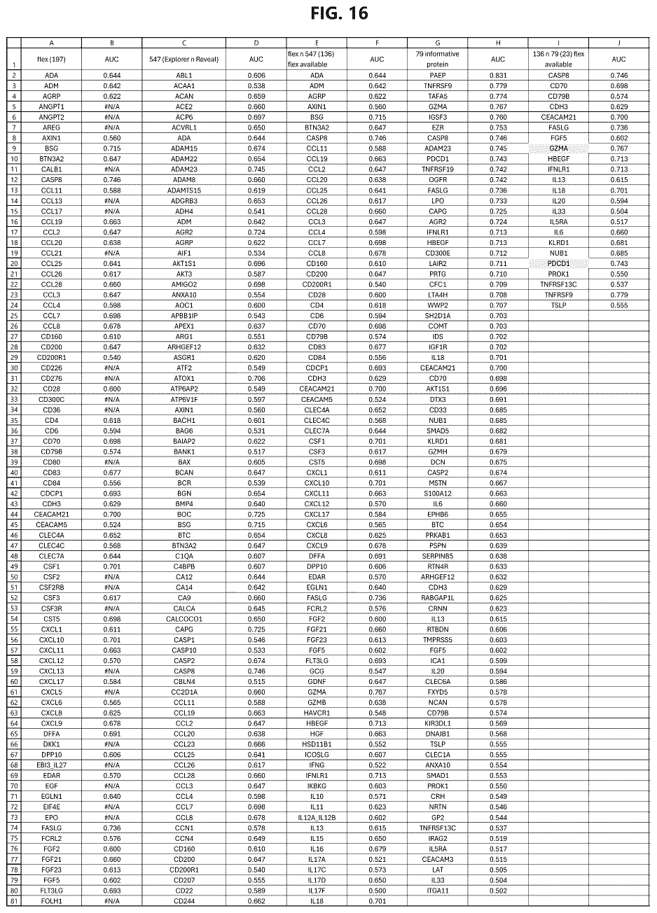

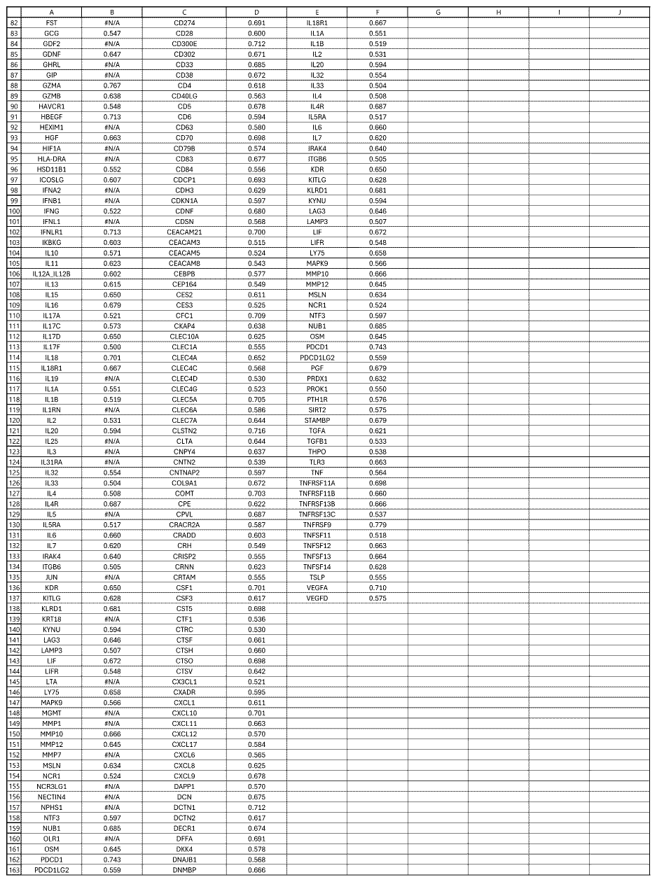

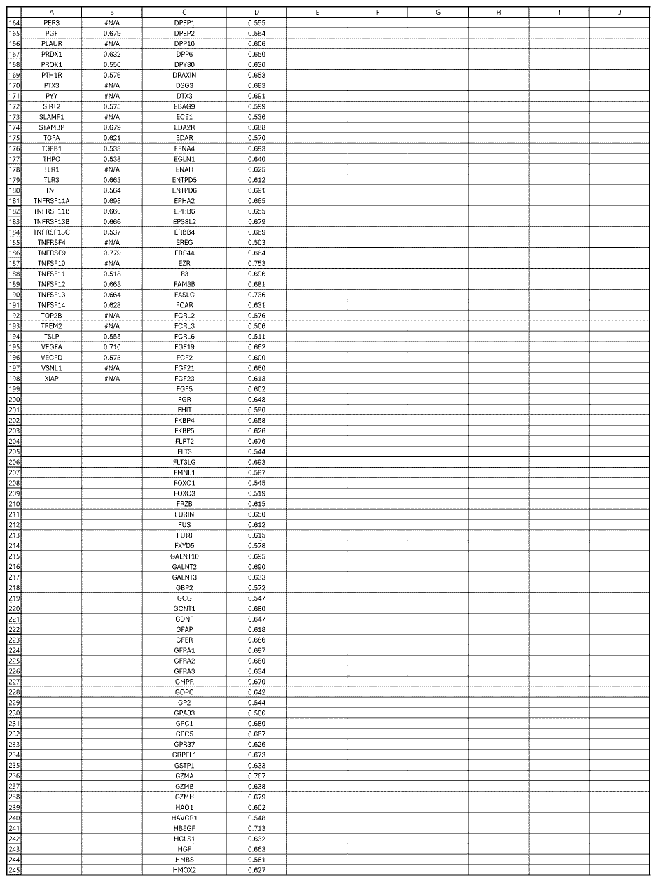

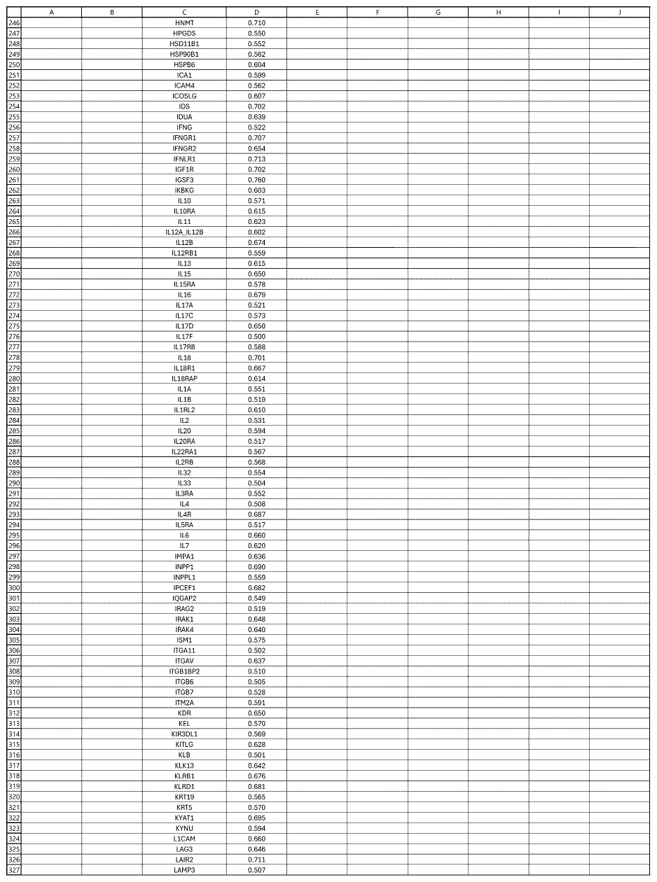

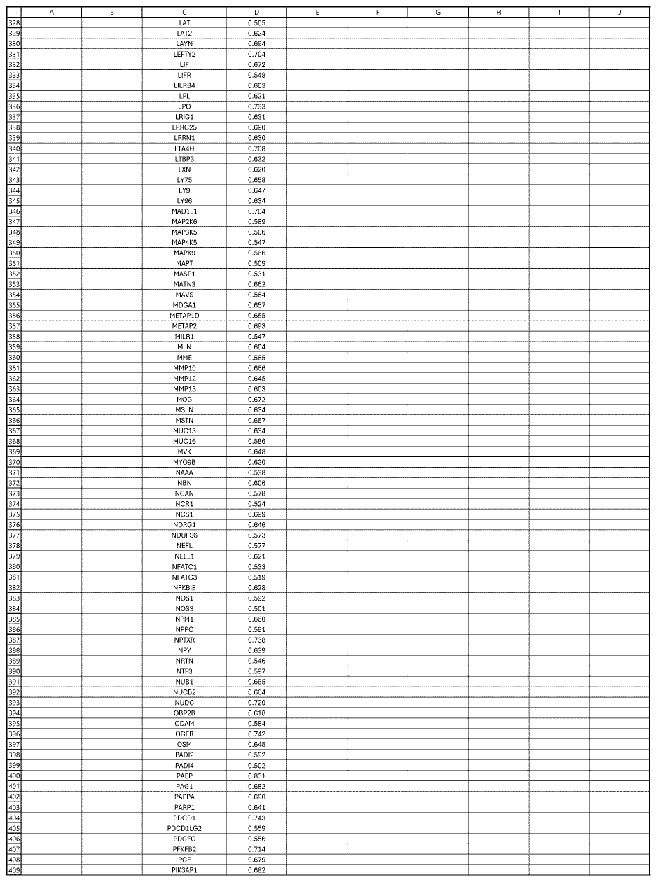

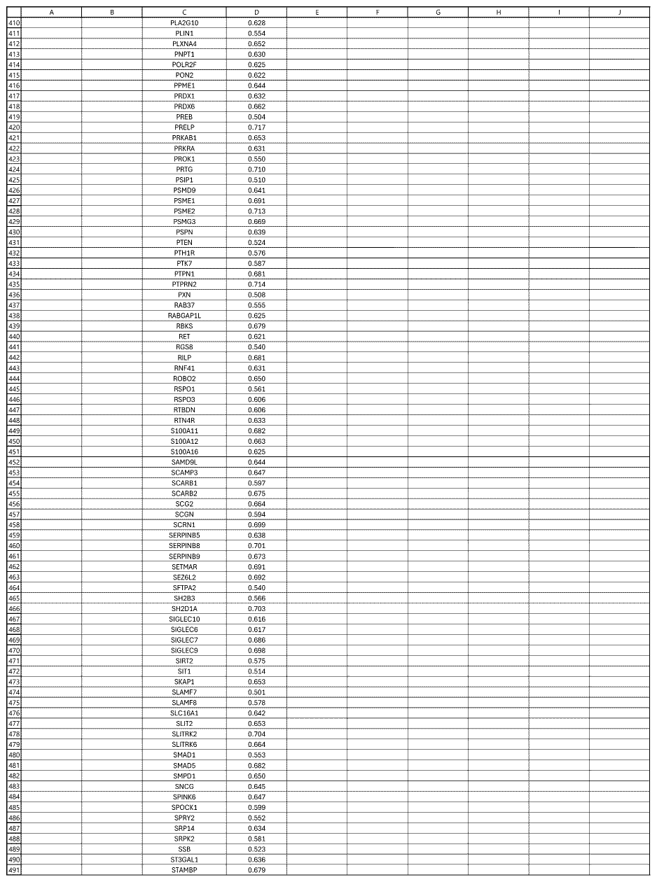

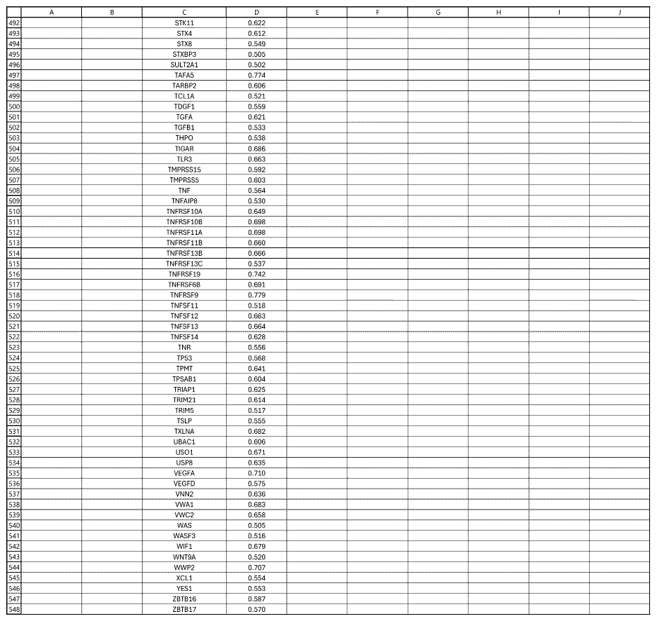

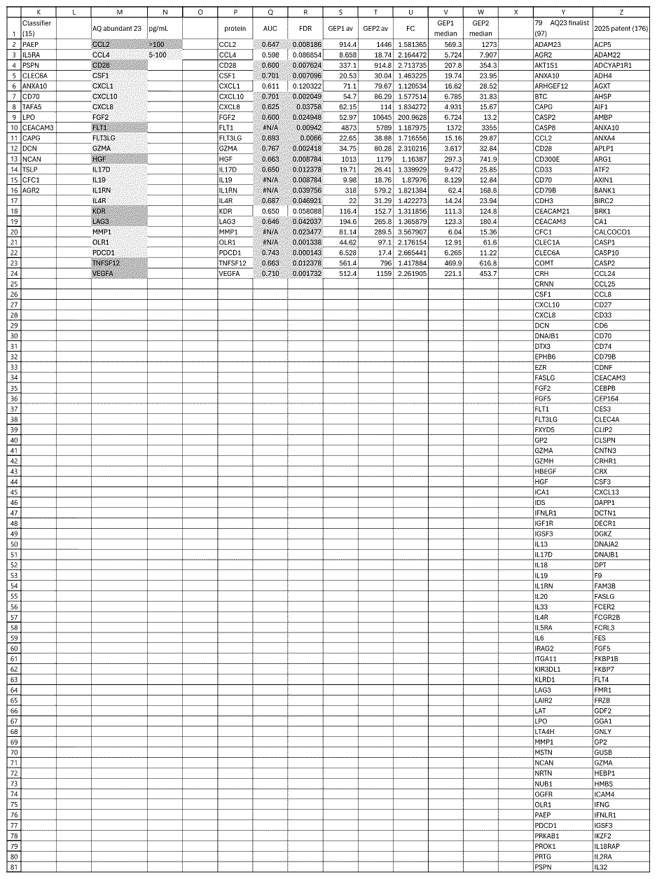

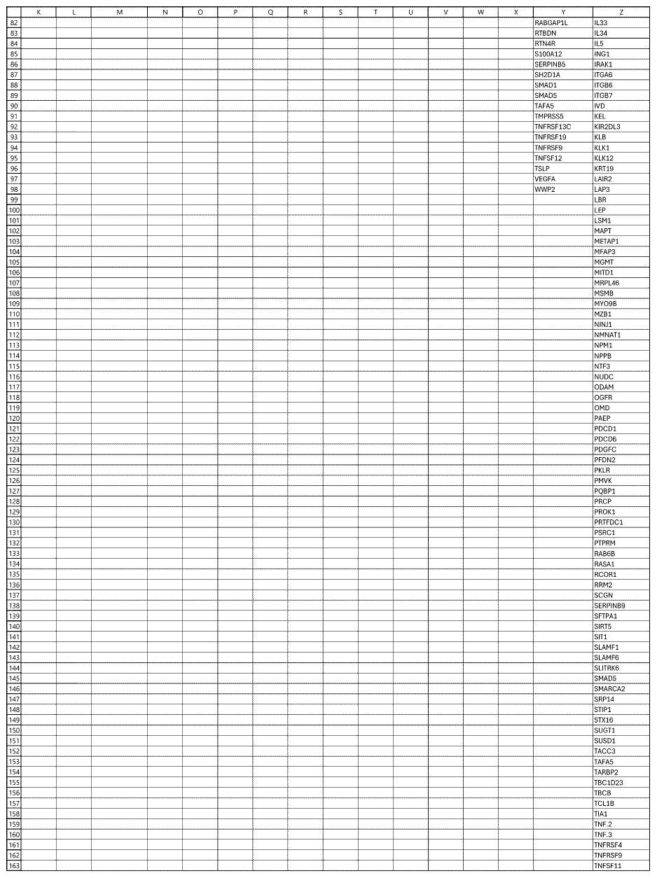

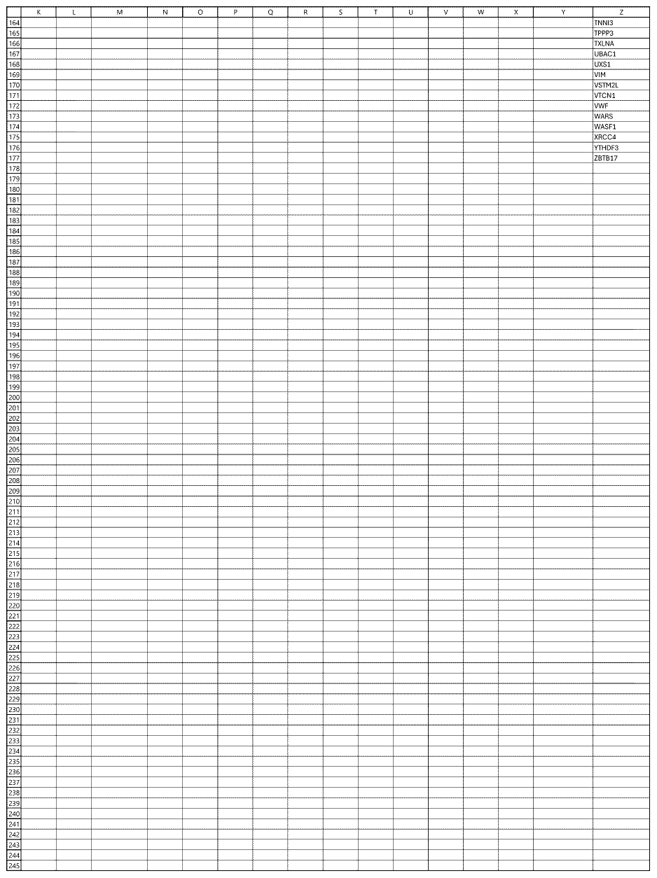

The trained 18-protein logistic regression classifier was applied to the 53 GEP-unknown AH samples to generate predicted metastatic risk classifications in the absence of tumor-derived molecular data. The predicted probability distribution for GEP-unknown samples is shown in FIG. 14A. Application of the 0.50 probability threshold to GEP-unknown samples yielded classification of 41 samples as potential GEP1 (PEP1, low metastatic risk) and 12 samples as potential GEP2 (PEP2, high metastatic risk), as shown in FIG. 14B. The distribution of predicted risk classifications in the GEP-unknown cohort was proportionally consistent with the distribution observed in the GEP-classified training cohort. Clinical validation demonstrated that PEP2-classified samples exhibited larger mean tumor volumes compared to PEP1-classified samples (356.5+385.6 mm³ versus 85.18±100.3 mm³; p=0.013), consistent with the known association between GEP2 classification and greater tumor burden. These findings support the biological relevance of the aqueous humor-derived protein expression classifications generated in the absence of tumor biopsy data and demonstrate the utility of the disclosed classifier for metastatic risk stratification in patients who have not undergone tumor fine-needle aspiration biopsy.

### Example V—Aqueous Humor Proteomics Enables GEP-Equivalent Metastatic Risk Stratification in Uveal Melanoma, Including Eyes without Tumor Biopsy

Uveal melanoma is an eye cancer where predicting the likelihood of tumor spread usually requires an invasive biopsy from inside the eye—a procedure that many patients cannot undergo. Provided herein is the identification of proteins present in the eye's aqueous humor that carry the same risk information as tumor tissue. By analyzing these proteins using machine learning, a highly accurate, biopsy-free test was developed that identifies patients at a higher risk of aggressive disease. This liquid biopsy can make molecular risk prediction accessible to more patients, facilitating earlier and more personalized care.

### Purpose

Current prognostication for uveal melanoma (UM) relies on gene expression profiling (GEP) of tumor tissue obtained via invasive fine-needle aspiration biopsy (FNAB). Yet, up to ⅓ of patients do not undergo tumor biopsy due to anatomic, clinical, or psychosocial limitations. Building on the prior work showing strong concordance between aqueous humor (AH) proteomics and tumor-derived GEP, it was evaluated whether an AH-based classifier could extend GEP-equivalent risk assessment to diagnostic UM eyes lacking tumor GEP.

### Methods

AH samples were collected from 114 UM patients prior to treatment. The cohort included a training set with confirmed tumor GEP results (n=69: 45 GEP1, 24 GEP2) and an application set lacking GEP information (n=45: GEP NA). Proteomic profiling and machine learning feature selection refined the previous signature to a 13-protein classifier of CX3CL1, IDS, IL1B, NCAN, ODAM, PAEP, PSPN, RABGAP1L, SH2D1A, SMAD1, SMAD5, TNFRSF9 and WAS. The model was trained on the GEP-known cohort and then applied to the GEP NA cohort to generate predicted molecular risk. Clinical relevance was evaluated by comparing tumor characteristics between risk groups.

### Results

In the training set, the 13-protein signature exhibited excellent discriminative power, achieving an area under the curve (AUC) of 1.0 (internal performance). This signature stratified the 45 GEP NA patients into 31 potential GEP1 (PEP1) cases and 14 potential GEP2 (PEP2) cases. Clinical validation indicated that patients predicted to be PEP2 (high-risk) had significantly larger tumor volumes compared to those predicted to be low risk. (Mean 240.9 vs 80.32 mm³; P=0.013). This distribution mirrored the phenotype of the training set, where confirmed GEP2 tumors exhibited significantly larger volumes compared to GEP1 (Mean 599.2 vs 448.3 mm³; P=0.015). No other clinical parameters differed significantly between risk groups in either cohort, consistent with the biological differences typically associated with GEP classes.

### CONCLUSION

A refined AH protein signature was validated that accurately predicts GEP-associated risk. It was demonstrated that this minimally invasive tool successfully identifies a GEP-equivalent high-risk molecular phenotype among patients without tissue biopsy, characterized by advanced tumor burden consistent with confirmed GEP2 cases. This liquid biopsy offers a robust surrogate for prognostication when surgical biopsy is inconclusive or not performed.

All publications, patents, and patent applications, sequences represented by sequence reference numbers/database names or gene/protein name, websites and other published materials referred to throughout the disclosure herein are herein incorporated by reference to the same extent as if each individual publication, patent, or patent application, sequence, websites and other published materials was specifically and individually indicated to be incorporated by reference. In the event that the definition of a term incorporated by reference conflicts with a term defined herein, this specification shall control.

What is claimed is:

1. A method for stratifying metastatic risk in a subject having uveal melanoma, the method comprising:
a) obtaining an aqueous humor sample from the subject;
b) quantifying protein expression levels in the aqueous humor sample to generate protein expression values for a plurality of proteins;
c) applying a machine learning classifier model to the protein expression values of a panel of protein biomarkers to generate a metastatic risk score, wherein the panel comprises at least three protein biomarkers, at least 5 biomarkers or at least 8 biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, ODAM, RABGAP1L, SH2D1A, SMAD1, SMAD5 and WAS, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma; and
d) classifying the subject as having high metastatic risk or low metastatic risk based on the metastatic risk score.

2. The method of claim 1, wherein the aqueous humor sample is collected from the subject prior to any therapeutic intervention for uveal melanoma.

3. The method of claim 1, wherein the aqueous humor sample is obtained by clear corneal paracentesis.

4. The method of claim 1, wherein b) protein expression levels are quantified using a multiplex proximity extension assay, electrochemiluminescence, Luminex multiplex immunoassay, ELISA, mass spectrometry, single molecule array, or an aptamer-based assay and optionally comprising generating normalized protein expression values and/or extrapolated absolute concentration values in picograms per milliliter derived by regression-based calibration using reference concentration values.

5. The method of claim 4, wherein the multiplex proximity extension assay is performed using a dual-platform calibration comprising a next generation sequencing-based analysis and a quantitative polymerase chain reaction-based analysis.

6. The method of claim 1, wherein the panel of protein biomarkers comprises PAEP, SMARCA2, FAM3B, KEL, SLITRK6, ANXA10, TNFRSF9, ADCYAP1R1, DECR1, GDF2, and PDCD1 or comprises CX3CL1, IDS, IL1B, NCAN, ODAM, PAEP, PSPN, RABGAP1L, SH2D1A, SMAD1, SMAD5, TNFRSF9 and WAS.

7. The method of claim 1, wherein the panel of protein biomarkers comprises PAEP, IL5RA, PSPN, CLEC6A, ANXA10, CD70, TAFA5, LPO, CEACAM3, CAPG, DCN, NCAN, TSLP, CFC1, and AGR2.

8. The method of claim 1, wherein the panel of protein biomarkers comprises TNFRSF9, CD70, TNFRSF13C, PROK1, CEACAM21, PDCD1, GZMA, CCL2, IL6, FGF2, CXCL10, CXCL8, IL17D, KLRD1, CD28, CD79B, FLT3LG, and HBEGF.

9. The method of claim 1, wherein the machine learning classifier model is a logistic regression model.

10. The method of claim 1, wherein the machine learning classifier model is trained using a feature selection process selected from the group consisting of least absolute shrinkage and selection operator (LASSO) regression, Random Forest, support vector machine-recursive feature elimination (SVM-RFE), and combinations thereof.

11. The method of claim 1, wherein classifying the subject comprises identifying the subject as having high metastatic risk when the metastatic risk score exceeds a predetermined probability threshold and identifying the subject as having low metastatic risk when the metastatic risk score is below the predetermined probability threshold.

12. The method of claim 11, wherein the predetermined probability threshold is determined using Youden's index.

13. The method of claim 1, wherein the subject has not undergone fine-needle aspiration biopsy of the uveal melanoma tumor.

14. The method of claim 1, further comprising administering a treatment to the subject based on the classification, wherein the treatment is selected from the group consisting of surgery, radiation therapy, plaque brachytherapy, photocoagulation, chemotherapy, immunotherapy, thermotherapy, and combinations thereof.

15. A method for generating a metastatic risk classifier for uveal melanoma, the method comprising:
a) obtaining aqueous humor samples from a training cohort of subjects having uveal melanoma, wherein the training cohort includes subjects with known gene expression profile classifications;
b) quantifying protein expression levels in the aqueous humor samples;
c) selecting a panel of protein biomarkers from the quantified protein expression levels by applying at least one feature selection method selected from the group consisting of LASSO regression, Random Forest, and support vector machine-recursive feature elimination, wherein the selected protein biomarkers are differentially expressed between low-risk and high-risk uveal melanoma tumors; and
d) training a machine learning classifier model on the protein expression values of the selected panel to generate a metastatic risk classifier, wherein the machine learning classifier is trained on aqueous humor protein expression values from a training cohort of subjects having uveal melanoma, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma.

16. The method of claim 15, wherein c) comprises applying each of LASSO regression, Random Forest, and support vector machine-recursive feature elimination, and selecting protein biomarkers identified as informative by all three feature selection methods.

17. The method of claim 15, wherein the machine learning classifier model is validated using five-fold stratified cross-validation.

18. The method of claim 15, further comprising administering a treatment to the subject based on the classification, wherein the treatment is selected from the group consisting of surgery, radiation therapy, plaque brachytherapy, photocoagulation, chemotherapy, immunotherapy, thermotherapy, and combinations thereof.

19. A diagnostic kit for stratifying metastatic risk in a subject having uveal melanoma, the kit comprising:
a) a collection device configured to receive an aqueous humor sample from the subject;
b) an assay reagent system configured to quantify protein expression levels in the aqueous humor sample;
c) a panel of detection reagents for protein biomarkers comprising at least three protein biomarkers selected from the group consisting of PAEP, TBCB, PDCD1, SMARCA2, ADCYAP1R1, TNFRSF9, FAM3B, SLITRK6, ZBTB17, KEL, GDF2, ANXA10, SLAMF1, DECR1, CD70, IL5RA, PSPN, CLEC6A, TAFA5, LPO, CEACAM3, CAPG, DCN, NCAN, TSLP, CFC1, AGR2, TNFRSF13C, PROK1, CEACAM21, GZMA, CCL2, IL6, FGF2, CXCL10, CXCL8, IL17D, KLRD1, CD28, CD79B, FLT3LG, HBEGF, CX3CL1, IDS, IL1B, ODAM, RABGAP1L, SH2D1A, SMAD1, SMAD5 and WAS; and
d) a non-transitory computer-readable storage medium storing instructions which, when executed by a processor, cause the processor to:
i) receive protein expression values generated by the assay reagent system;
ii) apply a machine learning classifier model trained on aqueous humor samples from subjects with known uveal melanoma metastatic risk classifications to the protein expression values to generate a metastatic risk score, and wherein the machine learning classifier is trained on either A) gene expression profile (GEP) classification data for the training cohort, wherein the GEP classification data comprises GEP1 (low metastatic risk) and GEP2 (high metastatic risk) designations derived from tumor-derived gene expression profiling, or B) clinical metastatic outcome data for the training cohort, wherein the clinical metastatic outcome data comprises observed development or absence of metastatic disease in the training cohort subjects following diagnosis of uveal melanoma; and
iii) output the metastatic risk score for stratifying the subject as having high metastatic risk or low metastatic risk.

20. The diagnostic kit of claim 19, wherein the panel of detection reagents comprises detection reagents for at least the protein biomarkers PAEP, SMARCA2, FAM3B, KEL, SLITRK6, ANXA10, TNFRSF9, ADCYAP1R1, DECR1, GDF2, and PDCD1.
